SEQUENCE LISTING (8 sequences)

SEQ ID NO: 1 - DNA, 621 bp, Saccharomyces cereviseae
  CDS (1)...(621): *
atgagctttatggatcaaatcccaggaggaggaaattatccaaaactcccagtagaatgccttcctaacttcccgatccaaccatctttgaccttcagaggtagaaatgactcgcataaactgaaaaactttatctccgaaataatgttaaacatgtctatgatatcttggccgaatgatgccagtcgtattgtgtactgcagaagacatttattaaaccccgctgctcagtgggctaatgactttgtacaagaacaaggtatacttgaaataacattcgacacattcatacaaggattatatcagcatttctataagccaccagatatcaataaaatctttaatgcaatcacgcaactttccgaagctaaacttggtattgagcgtctcaaccaacgattcagaaagatttgggacagaatgccaccagacttcatgaccgaaaaagctgccataatgacatatactaggctattgacaaaggaaacctataatattgtcagaatgcacaaaccagagacattaaaagacgccatggaagaggcttaccagacaactgcactaactgaaagattcttcccaggattcgaacttgatgctgatggagacactatcatcggt

SEQ ID NO: 2 - protein, 207 aa, Saccharomyces cereviseae
MSFMDQIPGGGNYPKLPVECLPNFPIQPSLTFRGRNDSHKLKNFISEIMLNMSMISWPNDASRIVYCRRHLLNPAAQWANDFVQEQGILEITFDTFIQGLYQHFYKPPDINKIFNAITQLSEAKLGIERLNQRFRKIWDRMPPDFMTEKAAIMTYTRLLTKETYNIVRMHKPETLKDAMEEAYQTTALTERFFPGFELDADGDTIIG

SEQ ID NO: 3 - DNA, 621 bp, Artificial Sequence
  CDS (1)...(621)
  Computer Generated Sequence
atgtcattcatggaccagattccgggcgggggtaactatcctaaattgccagtagaatgtttgccgaattttcccattcaaccaagtctgacctttcgcgggagaaacgatagccacaaactgaagaatttcattagcgagattatgctcaacatgtcgatgatctcttggcctaacgatgcgtctagaattgtgtactgccgtcgtcatttacttaatccagctgctcagtgggctaatgactttgtgcaagaacagggtattctcgagattacgttcgatacatttatccaggggctgtatcaacacttttataaaccgcctgatatcaataaaatctttaacgccatcacgcagctgtccgaggcaaaattaggcattgaacgtctgaatcaacggtttcggaaaatttgggatcgcatgccaccagatttcatgacagaaaaggccgcaattatgacgtatacccggttactgacgaaagagacctataatattgtacgtatgcataagccggagaccctgaaagatgcgatggaggaagcctaccagaccactgcccttaccgaacgattcttccctggctttgaactggacgcggacggagataccatcataggc

SEQ ID NO: 4 - protein, 207 aa, Artificial Sequence; identical to SEQ ID NO: 2
  Computer Generated Sequence

SEQ ID NO: 5 - DNA, 621 bp, Artificial Sequence
  CDS (1)...(621)
  Computer Generated Sequence
atgagtttcatggaccagattccgggtggtggtaactaccctaaactgccggttgaatgcctgccgaactttccgatccagcctagcctgacctttcgtggtcgtaacgatagccacaaacttaaaaacttcattagcgaaatcatgctgaacatgagtatgatcagctggccgaatgacgctagccgtatcgtttactgtcgtcgtcacctgcttaaccccgctgcgcaatgggctaatgacttcgttcaggaacagggtatcctggagatcacctttgacaccttcatccagggcctgtaccagcacttctataaaccgcctgacattaacaaaatcttcaacgcgatcacccaactgagcgaggcgaaactgggtatcgaacgtctgaaccagcgtttccgaaaaatttgggaccgtatgccgcccgacttcatgaccgaaaaagctgcgatcatgacctacacccgtctgctgactaaagaaacctacaacatcgttcgtatgcacaaaccggaaacccttaaagacgctatggaagaagcataccagaccaccgctctgaccgaacgtttcttcccaggtttcgaacttgacgcggacggtgacaccatcatcggt

SEQ ID NO: 6 - protein, 207 aa, Artificial Sequence; identical to SEQ ID NO: 2
  Computer Generated Sequence

SEQ ID NO: 7 - DNA, 693 bp, HIV-1
  CDS (1)...(693)
cctatagtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttg

SEQ ID NO: 8 - protein, 231 aa, HIV-1
PIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

(19) United States
(12) Patent Application Publication
(10) Pub. No.: US 2007/0298503 A1
(43) Pub. Date: Dec. 27, 2007

(54) ANALYZING TRASLATIONAL KINETICS USING GRAPHICAL DISPLAYS OF TRANSLATIONAL KINETICS VALUES OF CODON PAIRS

(76) Inventors: Richard H. Lathrop, Irvine, CA (US); Joseph D. Kittle JR., Irvine, CA (US); G. Wesley Hatfield, Corona del Mar, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/744,751

(22) Filed: May 4, 2007

Related U.S. Application Data
(63) Continuation-in-part of application No. 11/505,781, filed on Aug. 16, 2006.
(60) Provisional application No. 60/746,466, filed on May 4, 2006.
(60) Provisional application No. 60/841,588, filed on Aug. 30, 2006.

(51) Int. Cl. G01N 33/48; G06T 11/00

(52) U.S. Cl. 436/34; 345/440

(57) ABSTRACT

Graphical displays are provided of translational kinetics values of codon pairs in a host organism plotted as a function of polypeptide-encoding nucleotide sequence. Such translational kinetics values of codon pair frequencies correspond to the predicted translational pausing properties of a codon pair in a host organism. The graphical displays provided reflect the relative over-representation or under-representation of each codon pair in an organism, thereby facilitating analysis of translational kinetics of an mRNA into polypeptide by comparing graphical displays of different codon pairs in sequences encoding the polypeptide. The graphical displays of translational kinetics values also can display codon pair properties on comparable numerical scales, thereby facilitating analysis of translational kinetics of an mRNA into polypeptide in different organisms by comparing comparably scaled graphical displays of the same or different codon pairs in sequences encoding the polypeptide. Also contemplated herein is the use of the graphical displays described herein for tracking the entire process of creating a refined polypeptide-encoding nucleotide sequence. In particular, additional translational kinetics graphical displays can be created to illustrate differences and/or similarities of translational kinetics of a polypeptide-encoding nucleotide sequence in which one or more codon pairs have been modified. Additionally, numerous translational kinetics graphical displays can be created to illustrate differences and/or similarities of translational kinetics of a polypeptide-encoding nucleotide sequence when expressed in two or more different organisms.

### RELATED APPLICATIONS

This application is a continuation-in-part of U.S. non-provisional application Ser. No. 11/505,781, filed Aug. 16, 2006, and also claims priority to U.S. provisional application Ser. No. 60/746,466, filed May 4, 2006, and U.S. provisional application Ser. No. 60/841,588, filed Aug. 30, 2006. These applications are incorporated by reference herein in their entirety.

### FEDERALLY SPONSORED RESEARCH

The work resulting in this invention was supported in part by National Science Foundation Grant No. IIS-0326037 and National Institutes of Health Grant No. STTR 1R41-AI-066758. The U.S. Government may therefore be entitled to certain rights in the invention.

### BACKGROUND

1. Field of the Invention

The present invention generally relates to a new discovery in the field of genetics regarding codon pair usage in organisms, and using codon pair translational kinetics information in graphical displays for analyzing, altering, or constructing genes; for purposes of expression in other organisms; or to study or modify the translational efficiency of at least portions of the genes.

2. Description of the Related Art

The expression of foreign heterologous genes in transformed organisms is now commonplace. A large number of mammalian genes, including, for example, murine and human genes, have been successfully inserted into single celled organisms. Despite the burgeoning knowledge of expression systems and recombinant DNA, significant obstacles remain when one attempts to express a foreign or synthetic gene in an organism. Often, a synthetic gene, even when coupled with a strong promoter, is inefficiently translated and produces a faulty protein, such as an improperly folded or otherwise non-functional protein. The same is frequently true of exogenous genes foreign to the expression organism. Even when the gene is translated such that recoverable quantities of the translation product are produced, the protein is often inactive, insoluble, aggregated, or otherwise different in properties from the native protein.

The protein coding regions of genes in all organisms are subject to a wide variety of functional constraints, some of which depend on the requirement for encoding a properly functioning protein, as well as appropriate translational start and stop signals. However, several features of protein coding regions have been discerned which are not readily understood in terms of these constraints: two important classes of such features are those involving codon usage and codon context.

It has been known for a considerable time that codon utilization is highly biased and varies considerably between different organisms. The possibility that biases in codon usage can alter peptide elongation rates has been widely discussed, but while differences in codon use are thought to be associated with differences in translation rates, direct effects of codon choice on translation have been difficult to demonstrate. Additional proposed constraints on codon usage patterns include maximizing the fidelity of translation and optimizing the kinetic efficiency of protein synthesis. Replacing rarely used codons with frequently used codons may improve protein expression.

Apart from the non-random use of codons, evidence indicates that codon/anticodon recognition is influenced by sequences outside the codon itself, a phenomenon termed “codon context.” Although the context effect has been recognized by previous researchers, the predictive value of most statistical rules relating to preferred nucleotides adjacent to codons is relatively low. This, in turn, has severely limited the utility of such nucleotide preference data for selecting codons to effect desired levels of translational efficiency.

In one study (U.S. Pat. No. 5,082,767), it was found that codon pair utilization was biased, reflecting over-representation or under-representation of various codon pairs relative to expected codon pair frequencies. This codon utilization bias varies in different types of organisms. Using chi-squared analysis, U.S. Pat. No. 5,082,767 showed that over-represented codon pairs of a known nucleotide sequence in its native organism could be identified, and these chi-squared values could be plotted for codons encoding protein regions. However, a graphical representation of chi-squared values such as that of U.S. Pat. No. 5,082,767 does not reflect the relative degree by which codon pairs are over-represented or under-represented. In addition, the magnitude of chi-squared values calculated according to U.S. Pat. No. 5,082,767 varies from calculation to calculation and from organism to organism depending on the amount of data input into the chi-squared analysis. These shortcomings result in graphical representations that are difficult to use, both in terms of using the graph to evaluate possible modification of a codon sequence, and in terms of comparing the graphs for expression in different organisms. In particular, scaling differences from graph-to-graph increases the ambiguity of evaluating sequence modifications and/or expression in different organisms.

Such chi-squared values have been used to estimate translational kinetics for proteins. However, such estimates are only a first approximation, and do not represent true predictions of translational kinetics. Heretofore, shortcomings in chi-squared based predictions of translational kinetics have not been appreciated, and, thus, methods for improving the translational kinetics predictive value of codon pairs have not been explored.

### SUMMARY

In order to improve upon the shortcomings in the art, provided herein are graphical displays of translational kinetics values for codon pairs in a host organism plotted as a function of polypeptide or polypeptide-encoding nucleotide sequence. Such translational kinetics values can be based on: values of observed versus expected codon pair frequencies in a host organism; empirically measured translational pause properties; observed presence and/or recurrence of codon pairs at known or predicted transcriptional pause sites; or other methods known to those skilled in the art. The graphical displays provided herein reflect translational kinetics for each codon pair in a polypeptide-encoding nucleotide sequence to be expressed in an organism, thereby facilitating analysis of translational kinetics of an mRNA into polypeptide by comparing graphical displays of different codon pairs in sequences encoding the polypeptide. The graphical displays of translational kinetics values also display codon pair preferences on comparable numerical scales, thereby facilitating analysis of translational kinetics of an mRNA into polypeptide in different organisms by comparing comparably scaled graphical displays of the same or different codon pairs in sequences encoding the polypeptide.

In addition to displaying codon pair utilization information for a gene in its native organism, as in U.S. Pat. No. 5,082,767, also contemplated herein is the use of the graphical displays described herein for tracking the entire process of creating a synthetic polypeptide-encoding nucleotide sequence. In particular, sets of translational kinetics graphical displays can be created to illustrate differences and/or similarities of translational kinetics of a polypeptide-encoding nucleotide sequence in which one or more codon pairs have been modified. Additionally, numerous translational kinetics graphical displays can be created to illustrate differences and/or similarities of translational kinetics of a polypeptide-encoding nucleotide sequence when expressed in two or more different organisms.

Also provided herein are methods of determining improved translational kinetics values. Translational kinetics values based solely on observed codon pair frequency versus expected codon pair frequency can be used as first approximations of translational kinetics of a polypeptide-encoding nucleotide sequence. However, such values are not true predictors of translational kinetics, and methods are provided herein to improve the translational kinetics value for a codon pair. As provided herein, the translational kinetics value for a codon pair in a host organism can be refined or replaced based on translational kinetic information, and the improved translational kinetics value can be used in graphical displays and methods of predicting translational kinetics. The various types of codon pair translational kinetics information that can be used in refining or replacing a translational kinetics value for a codon pair include, for example, values of observed versus expected codon pair frequencies in a particular organism, normalized values of observed versus expected codon pair frequencies in a particular organism, the degree to which observed versus expected codon pair frequency values are conserved in related proteins across two or more species, the degree to which observed versus expected codon pair frequency values are conserved at predicted pause sites such as boundaries between autonomous folding units in related proteins across two or more species, the degree to which codon pairs are conserved at predicted pause sites across different proteins in the same species, and empirical measurement of translational kinetics for a codon pair.

The graphical displays and methods provided herein can be used in a variety of applications provided herein, and additional applications that will be readily apparent to one skilled in the art. For example, the graphical displays and methods provided herein can be used in methods of genetic engineering, in development of biologics such as therapeutic biologics, preparation of immunological reagents including vaccines, preparation of serological diagnostic products, and additional protein production technologies known in the art.

In one embodiment, provided are methods of analyzing translational kinetics of an mRNA into polypeptide encoded by a heterologous gene in a host organism by providing translational kinetics values for codon pairs in a host organism, generating a first graphical display of the translational kinetics values of actual codon pairs of an original polypeptide-encoding nucleotide sequence of a heterologous gene as a function of codon position, providing a modified nucleotide sequence encoding the same polypeptide as the original nucleotide sequence, generating a second graphical display of the translational kinetics values of the codon pairs of the modified polypeptide-encoding nucleotide sequence as a function of codon position, and comparing said first and second graphical displays to predict the translational kinetics of the polypeptide encoded by the modified polypeptide-encoding nucleotide sequence relative to the unmodified polypeptide-encoding nucleotide sequence. In some embodiments, the translational kinetics values are based, at least in part, on normalized chi-squared values of observed codon pair frequency versus expected codon pair frequency in the host organism. In some embodiments, the translational kinetics values are based, at least in part, on normalized chi-squared values of observed codon pair frequency versus expected codon pair frequency in the host organism, based on nucleotide sequence data that has been at least partially clustered and weighted in performing the chi-squared calculation. In some embodiments, the translational kinetics values are based, at least in part, on normalized chi-squared values of observed codon pair frequency versus expected codon pair frequency in a group of organism types, typically where the group includes the host organism. In some embodiments, the translational kinetics values are based, at least in part, on an empirical measurement of the translational kinetics of a codon pair in the host organism. In some embodiments, the translational kinetics values are based, at least in part, on determination of a translational kinetics value that is conserved across two or more species at a boundary location between autonomous folding units of a protein present in the two or more species, wherein the group of two or more species includes the host organism. In some embodiments, the translational kinetics values are based, at least in part, on determination of a normalized value of observed codon pair frequency versus expected codon pair frequency conserved across two or more species at a boundary location between autonomous folding units of a protein present in the two or more species, wherein the group of two or more species includes the host organism. In some embodiments, the translational kinetics values are based, at least in part, on determination of a translational kinetics value that is positionally conserved across two or more species for a protein present in the two or more species, wherein the group of two or more species includes the host organism. In some embodiments, the translational kinetics values are based, at least in part, on determination of a normalized value of observed codon pair frequency versus expected codon pair frequency that is positionally conserved across two or more species for a protein present in the two or more species, wherein the group of two or more species includes the host organism. In some embodiments, the translational kinetics values are based, at least in part, on determination of a codon pair conserved across two or more proteins of the host organism at boundary locations between autonomous folding units of the two or more proteins. In some embodiments, the graphical display includes an abscissa that delineates nucleotide position of a polypeptide-encoding nucleotide sequence. In some embodiments, the graphical display includes an ordinate that contains negative and positive values, where the zero value corresponds to the mean chi-squared value of observed versus expected codon pair frequencies for genes native to the host organism. In some embodiments, the scale of the ordinate of the graphical display is in units of standard deviations. In some embodiments, the original polypeptide-encoding nucleotide sequence and the modified polypeptide-encoding nucleotide sequence both encode the same amino acid sequence. In some embodiments, the original polypeptide-encoding nucleotide sequence and the modified polypeptide-encoding nucleotide sequence encode different amino acid sequences. In some embodiments, the original polypeptide-encoding nucleotide sequence is modified such that the second graphical display contains a predicted translational pause at a codon pair site that is not predicted to be a translational pause in the first graphical display, where this translational pause site is located between two autonomous folding units of a protein. In some embodiments, the original polypeptide-encoding nucleotide sequence is modified such that the second graphical display is not predicted to have a translational pause at a codon pair site that is predicted to be a translational pause in the first graphical display, where the site of the predicted translational pause is located within an autonomous folding unit of a protein. In some embodiments, the original polypeptide-encoding nucleotide sequence is modified such that the second graphical display more closely resembles the translational kinetics of the mRNA into polypeptide in its native host organism relative to the unmodified polypeptide-encoding nucleotide sequence. In some such embodiments, the original polypeptide-encoding nucleotide sequence is modified such that the second graphical display is predicted to contain a translational pause at a codon pair site that is not predicted to be a translational pause in the first graphical display, where a graphical display of wild type gene expression in the native host organism indicates that this codon pair site is predicted to be a translational pause. In some such embodiments, the original polypeptide-encoding nucleotide sequence is modified such that the second graphical display is predicted to not contain a translational pause at a codon pair site that is predicted to be a translational pause in the first graphical display, where a graphical display of wild type gene expression in the native host organism indicates that this codon pair site is predicted to not be a translational pause. In some embodiments, the original polypeptide-encoding nucleotide sequence is modified such that the second graphical display contains substantially no codon pairs having a z score greater than 5 standard deviations. In some embodiments, the original polypeptide-encoding nucleotide sequence is modified such that the second graphical display contains substantially no codon pairs having a z score greater than 4 standard deviations. In some embodiments, the original polypeptide-encoding nucleotide sequence is modified such that the second graphical display contains substantially no codon pairs having a z score greater than 3 standard deviations. In some embodiments, the original polypeptide-encoding nucleotide sequence is modified such that the second graphical display contains substantially no codon pairs having a z score greater than 2 standard deviations. In some embodiments, the original polypeptide-encoding nucleotide sequence is modified such that the second graphical display contains substantially no codon pairs that are over-represented by more than 5 standard deviations. In some embodiments, the original polypeptide-encoding nucleotide sequence is modified such that the second graphical display contains substantially no codon pairs that are over-represented by more than 4 standard deviations. In some embodiments, the original polypeptide-encoding nucleotide sequence is modified such that the second graphical display contains substantially no codon pairs that are over-represented by more than 3 standard deviations. In some embodiments, the original polypeptide-encoding nucleotide sequence is modified such that the second graphical display contains substantially no codon pairs that are over-represented by more than 2 standard deviations. In some embodiments, the translational kinetics values are chi-squared 2 values. In some embodiments, the translational kinetics values are chi-squared 3 values. In some embodiments, the translational kinetics values are normalized chi-squared values. In some embodiments, the original polypeptide-encoding nucleotide sequence is a synthetic gene designed to be formed from a plurality of partially overlapping segments that hybridize under conditions that disfavor hybridization of non-adjacent segments. In some embodiments, the modified polypeptide-encoding nucleotide sequence is a synthetic gene designed to be formed from a plurality of partially overlapping segments that hybridize under conditions that disfavor hybridization of non-adjacent segments. In some embodiments, the original polypeptide-encoding nucleotide sequence is modified with reference to the effect of the modification on one or more characteristics selected from the group consisting of melting temperature gap between oligonucleotides of synthetic gene, average codon usage, average codon pair chi-squared frequency, absolute codon usage, absolute codon pair frequency, maximum usage in adjacent codons, occurrence of a Shine-Delgarno sequence, occurrence of 5 consecutive G's or 5 consecutive C's, occurrence of a long exactly repeated subsequence, occurrence of a cloning restriction site, occurrence of a user-prohibited sequence, codon usage of a specific codon above user-specified limit, and occurrence of an out of frame stop codon.

Also provided herein are methods of analyzing translational kinetics of an mRNA into polypeptide encoded by a gene in a non-native host organism by providing translational kinetics values for codon pairs in a first host organism, generating a first graphical display of the provided translational kinetics values of actual codon pairs provided for a polypeptide-encoding nucleotide sequence of a gene as a function of codon position, wherein the gene is native to the first host organism, providing translational kinetics values for codon pairs in a second host organism, wherein the polypeptide-encoding nucleotide sequence of the gene is not native to the second organism, generating a second graphical display of the second translational kinetics values of the codon pairs for the polypeptide-encoding nucleotide sequence of the gene as a function of codon position, and comparing said first and second graphical displays to predict the translational kinetics in the first host organism relative to the translational kinetics in the second host organism. Some methods further include modifying the polypeptide-encoding nucleotide sequence of the gene, generating a third graphical display of the translational kinetics values for the codon pairs of the modified polypeptide-encoding nucleotide sequence of the gene as a function of codon position, and comparing said first and/or second graphical displays to the third graphical display to predict translational kinetics of the mRNA into polypeptide encoded by the modified polypeptide-encoding nucleotide sequence relative to the unmodified polypeptide-encoding nucleotide sequence. In some such methods, the polypeptide-encoding nucleotide sequence is modified such that the second graphical display more closely resembles translational kinetics of the mRNA into polypeptide in its native host organism. In some such embodiments, the original polypeptide-encoding nucleotide sequence is modified such that the second graphical display is predicted to contain a translational pause at a codon pair site that is not predicted to be a translational pause in the first graphical display, where a graphical display of wild type gene expression in the native host organism indicates that this codon pair site is predicted to be a translational pause. In some such embodiments, the original polypeptide-encoding nucleotide sequence is modified such that the second graphical display is predicted to not contain a translational pause at a codon pair site that is predicted to be a translational pause in the first graphical display, where a graphical display of wild type gene expression in the native host organism indicates that this codon pair site is predicted to not be a translational pause.

Also provided herein are sets of graphical displays of translational kinetics chi-squared values of observed versus expected codon pair frequencies in a host organism plotted as a function of polypeptide-encoding nucleotide sequence, including a first graphical display of translational kinetics values in a host organism of actual codon pairs of an original polypeptide-encoding nucleotide sequence of a heterologous gene as a function of codon position, and a second graphical display of the translational kinetics values in the host organism of codon pairs of a modified polypeptide-encoding nucleotide sequence of the heterologous gene as a function of codon position.

Also provided herein are methods of refining the predictive capability of a translational kinetics value of a codon pair in a host organism, by providing an initial translational kinetics value based on the value of observed codon pair frequency versus expected codon pair frequency for a codon pair in a host organism, providing additional translational kinetics data for the codon pair in the host organism, and modifying the initial translational kinetics value according to the additional codon pair translational kinetics data to generate a refined translational kinetics value for the codon pair in the host organism. In some methods, the additional translational kinetics data are selected from the group consisting of normalized chi squared values of observed codon pair frequency versus expected codon pair frequency in the host organism, an empirical measurement of the translational kinetics of the codon pair in the host organism, degree of conservation of translational kinetics value across two or more species at a boundary location between autonomous folding units of a protein present in the two or more species, wherein the group of two or more species includes the host organism, degree of positional conservation of translational kinetics value across two or more species for a protein present in the two or more species, wherein the group of two or more species includes the host organism, degree of conservation of translational kinetics value across two or more proteins of the host organism at a boundary location between autonomous folding units of the two or more proteins, and combinations thereof. In some methods, the modifying step further comprises modifying the translational kinetics value of a selected codon pair according to two or more types of translational kinetics data.

Also provided are methods of improving the predictive capability of a translational kinetics value of a codon pair in a host organism, by providing translational kinetics data for the codon pair in the host organism, and generating a translational kinetics value based, at least in part, on the provided translational kinetics data, wherein the codon pair translational kinetics data are selected from the group consisting of an empirical measurement of the translational kinetics of the codon pair in the host organism, degree of conservation of translational kinetics value across two or more species at a boundary location between autonomous folding units of a protein present in the two or more species, wherein the group of two or more species includes the host organism, degree of positional conservation of translational kinetics value across two or more species for a protein present in the two or more species, wherein the group of two or more species includes the host organism, degree of conservation of translational kinetics value across two or more proteins of the host organism at a boundary location between autonomous folding units of the two or more proteins, and combinations thereof. In some methods, the translational kinetics value is the observed codon pair frequency versus expected codon pair frequency. In some methods, the observed codon pair frequency versus expected codon pair frequency is normalized.

Also provided herein are methods of analyzing translational kinetics of an mRNA into polypeptide encoded by a heterologous gene in a host organism comprising providing the amino acid sequence of a heterologous gene; identifying amino acid sequences related to the amino acid sequence of the heterologous gene; aligning the related amino acid sequences with each other and with the amino acid sequence of the heterologous gene; determining the translational kinetics values of the codon pairs of the nucleotide sequence encoding each of the aligned amino acid sequences; generating a graphical display reflecting the alignment of the amino acid sequences and reflecting the translational kinetics values of the codon pairs of the nucleotide sequence encoding each of the aligned amino acid sequences; and identifying one or more locations in the aligned amino acid sequences in which translational kinetics values are conserved over most or all aligned amino acid sequences. In some such methods, the identifying step comprises identifying a predicted pause that is conserved over most or all aligned amino acid sequences. Similarly, provided herein are methods of generating a graphical display of conserved translational kinetics of related genes comprising providing the amino acid sequence of a selected gene; identifying amino acid sequences related to the amino acid sequence of the heterologous gene; aligning the related amino acid sequences with each other and with the amino acid sequence of the heterologous gene; determining the translational kinetics values of the codon pairs of the nucleotide sequence encoding each of the aligned amino acid sequences; and generating a graphical display reflecting the alignment of the amino acid sequences and reflecting the translational kinetics values of the codon pairs of the nucleotide sequence encoding each of the aligned amino acid sequences. Also provided herein are graphical displays generated by such methods. In on embodiment, a graphical display is provided that comprises a plurality of related amino acid sequences aligned with each other, wherein the depiction of the amino acid sequences also reflects the translational kinetics values of the codon pairs of the nucleotide sequence encoding the aligned amino acid sequences.

Computer usable medium having computer readable program code comprising instructions for performing any one of the herein provided methods also is provided herein. A computer readable medium containing software that, when executed, causes the computer to perform the acts of any one of the herein provided methods also is provided herein.

### BRIEF DESCRIPTION OF THE DRAWINGS

FIG. 1 depicts effects of Translational Engineering on Protein Expression Levels. FIG. 1A depicts Western blots of the Saccharomyces cereviseae retropransposon Ty3 Capsid protein expressed from codon optimized (see FIG. 1B), hot-rod (see FIG. 1C), and native (see FIG. 1D) genes induced at two arabinose concentrations in equal numbers of E. coli cells harvested at mid-log growth at 37° C. in LB broth. FIGS. 1B-E depict graphical displays of z scores of chi-squared values for codon pair utilization of nucleic acid sequences encoding the capsid of the Ty3 retrotransposon of S. cerevisiae, plotted as a function of codon pair position. FIG. 1B depicts a graphical display of the Escherichia coli expression of a nucleic acid sequence encoding the Ty3 capsid which has been modified to optimize codon usage for expression in E. coli. FIG. 1C depicts a graphical display of the E. coli expression of a nucleic acid sequence encoding the Ty3 capsid which has been modified to eliminate codon pairs that are over-represented in E. coli. FIG. 1D depicts a graphical display of the E. coli expression of the native nucleic acid sequence encoding the Ty3 capsid. FIG. 1E depicts a graphical display of the S. cereviseae expression of the native nucleic acid sequence encoding the Ty3 capsid.

FIG. 2 depicts graphical displays of z scores of chi-squared values for codon pair utilization of nucleic acid sequences encoding the capsid protein of the human immunodeficiency virus, HIV-1, and the capsid protein of the S. cereviseae retrotransposon, Ty3. (A) HIV-1. (B) Ty3. The ribbon structure of each protein is shown above the respective graphical display. The regions of the abscissa indicating the amino terminal and the carboxy terminal domains of each protein are indicated by brackets. The thick black horizontal lines identify the positions of alpha helices in each protein.

FIG. 3 depicts a flow chart of the process for refining a nucleotide sequence that encodes a polypeptide to be expressed. The general computational framework is described in “Multi-Queue Branch-and-Bound Algorithm for Anytime Optimal Search with Biological Applications,” Lathrop, R. H., Sazhin, A., Sun, Y., Steffen, N., Irani, S., pp. 73-82 in Proc. Intl. Conf. on Genome Informatics, Tokyo, Dec. 17-19, 2001, Genome Informatics 2001 (Genome Informatics Series No. 12), Universal Academy Press, Inc., which is incorporated in its entirety by reference.

FIG. 4 provides the nucleotide and amino acid sequences depicted in FIGS. 1 and 2 and described in Examples 1 and 2.

### DETAILED DESCRIPTION OF THE PREFERRED EMBODIMENTS

Provided herein are tools and methods for analyzing and designing gene sequences, especially to aid gene expression, protein solubility, protein folding and other desirable properties related to protein translation. In particular, provided are tools and methods for the rational manipulation of translational kinetics in gene expression, and ways for manipulating gene sequences so as to use a gene's graphical display from its native organism to assist in engineering the gene's expression in a heterologous host organism.

In particular, provided herein are graphical displays of translational kinetics values for codon pairs in a host organism plotted as a function of polypeptide or polypeptide-encoding nucleotide sequence. Such translational kinetics values can be based on values of observed versus expected codon pair frequencies in a host organism, empirically measured translational pause properties, observed presence and/or recurrence of codon pairs at known or predicted transcriptional pause sites, or other measures known to those skilled in the art. The graphical displays provided herein reflect translational kinetics for each codon pair in a polypeptide-encoding nucleotide sequence to be expressed in an organism, thereby facilitating analysis of translational kinetics of an mRNA into polypeptide by comparing graphical displays of different codon pairs in sequences encoding the polypeptide. The graphical displays of translational kinetics values also display codon pair preferences on comparable numerical scales, thereby facilitating analysis of translational kinetics of an mRNA into polypeptide in different organisms by comparing comparably scaled graphical displays of the same or different codon pairs in sequences encoding the polypeptide. In some embodiments, the graphical display is a depiction of aligned related sequences such as evolutionarily conserved sequences in different species, where the depiction of the sequence reflects the translational kinetics value of the codon pairs of each aligned sequence.

In addition to displaying codon pair utilization information for a gene in its native organism, also contemplated herein is the use of the graphical displays described herein for tracking the entire process of creating a refined polypeptide-encoding nucleotide sequence. In particular, additional translational kinetics graphical displays or sets of displays can be created to illustrate differences and/or similarities of translational kinetics of a polypeptide-encoding nucleotide sequence in which one or more codon pairs have been modified. Additionally, numerous translational kinetics graphical displays can be created to illustrate differences and/or similarities of translational kinetics of one or more polypeptide-encoding nucleotide sequences when expressed in two or more different organisms.

In addition to graphical displays described herein, also contemplated herein are methods for increasing the accuracy of translational kinetics value calculations. Chi-squared values describing the degree of representation of codon pairs in polypeptide-encoding nucleotide sequences have been used to estimate translation kinetics for proteins (see U.S. Pat. No. 5,082,767). However, such estimates are only a first approximation, and do not represent true predictions of translational kinetics. Heretofore, shortcomings in chi-squared based predictions of translational kinetics have not been appreciated, and, thus, methods for improving the translational kinetics predictive value of codon pairs have not been explored. The methods provided herein for improving translational kinetics predictive value of codon pairs include improving chi-squared calculations by clustering of redundant and/or related sequences of an organism and weighting the codon pairs within the clustered sequences according to the size of the cluster, calculation of generic chi-squared values for multiple organisms to increase the amount of data considered in the chi-squared value calculation, estimating translational kinetics values from the conservation of the presence or absence of certain codon pairs at certain places in one or more multiple sequence alignments of related genes from different organisms, estimating translational kinetics values from the conservation of the presence or absence of certain codon pairs at certain protein structural domain boundaries or interiors, and empirical measurement of codon pair translational step times.

Further provided herein are methods of modifying the translational kinetics of a polypeptide-encoding nucleotide sequence. In some embodiments, a modified polypeptide-encoding nucleotide sequence is designed to reduce the number of predicted translational pauses relative to the unmodified original polypeptide-encoding nucleotide sequence. In some embodiments, a modified polypeptide-encoding nucleotide sequence is designed to replace all codon pairs predicted to cause translational pauses with codon pairs not predicted to cause translational pauses. In some embodiments, a modified polypeptide-encoding nucleotide sequence is designed to preserve one or more, up to all, predicted translational pauses of the unmodified original polypeptide-encoding nucleotide sequence when expressed in its native organism. In some embodiments, a modified polypeptide-encoding nucleotide sequence is designed to insert a predicted translational pauses not present in the unmodified original polypeptide-encoding nucleotide sequence. In some embodiments, a modified polypeptide-encoding nucleotide sequence is designed to include one or more predicted translational pauses present in a related polypeptide that is native to the organism in which the modified polypeptide-encoding nucleotide sequence will be expressed.

It has been discovered that codon pair utilization is biased: some codon pairs are over-represented while others are under-representated relative to expected codon pair frequencies. The observed frequency of some codon pairs is many standard deviations higher than the expected abundance, and this over-representation is independent of single codon usage, dinucleotides, and amino acid pairs. This phenomenon is specific and directional; if the order of the codons in a pair is reversed, the degree of representation is unrelated to the original pair. This statistical aberration is not accounted for by abundance of the codons themselves, amino acid pair associations, dinucleotide abundances, or other factors. This statistical anomaly is present in all organisms tested, but the actual codon pairs in the over-represented group are different for each organism.

In vivo translation experiments reveal that over-represented codon pairs in a gene's open reading frame have the effect of slowing translation, where the greater the degree of over-representation results in a greater translational slowing. Translational slowing creates translational pauses. While not intending to be limited to any particular explanation of these observations, it is proposed that the tRNA molecules that bind to the A-site and P-site of a ribosome during the translation of a biased codon pair are poorly compatible for binding and peptide transfer on the surface of a ribosome, resulting in unfavorable translational kinetics. The importance of codon pair-dictated kinetics has been seen in an isolated system, where a single silent change in a codon resulted in a thirty-fold change in expression (Trinh et al., Mol. Immunol. (2004) 40:717-722).

Thus, consistent with the above, reference to a “native host organism” in which a gene is expressed refers to an organism in which a particular gene's native expression is adapted to utilize one or more cellular components for protein translation (e.g., ribosome or tRNA molecules). For example, a native host organism for a gene can be an organism from which the gene to be expressed originates, or a native host organism for a gene can be an organism in which a viral gene is expressed where the source virus is adapted to native gene expression in the organism.

Further, as used herein, the term “gene” is used in a non-limiting fashion, to include (at a minimum) a polynucleotide sequence encoding a particular desired polypeptide sequence, whether or not it includes untranslated regions, splice sites, promoters, and the like, and whether or not it encodes an entire protein or only a portion thereof. Similarly, the term “polypeptide” is used in a non-limiting fashion, to include peptide sequences that are relatively short (e.g., 10, 20, 30, or 50 amino acids) as well as those that are relatively long (hundreds of amino acids, or even more).

As used herein “translational kinetics” refers to the rate of ribosomal movement along messenger RNA during translation. Similarly, a “translational kinetics value” of a codon pair as used herein refers to a representation of the rate of ribosomal movement along a particular codon pair of messenger RNA during translation. For some codon pairs, a translational kinetics value can represent a predicted translational pause or slowing of the ribosome along the messenger RNA during translation.

Codon Pair Usage and Sequence Refinement

It is proposed herein that the presence of a pause or translation slowing codon pair can queue ribosomes back to the beginning of the coding sequence, thereby inhibiting further ribosome attachment to the message which can result in down-regulation of protein expression levels as the rate of translation initiation readily saturates and the slowest translation step becomes rate limiting. It is also proposed herein that the presence of a pause or translational slowing codon pair can stall or detach a ribosome. It is also proposed herein that the presence of a pause or translational slowing codon pair can expose naked mRNA, which is then subject to message degradation. It is also proposed herein that the presence of a pause or translational slowing codon pair can decouple translation from transcription, leading to protein expression failure. For these reasons and more, methods for analyzing and designing gene sequences for pauses or translational slowing have great utility.

Organism-specific codon usage and codon pair usage, and the presence of organism-specific pause sites, result in gene translation and expression that is highly adapted to its original host organism. For example, ribosomal pausing sites that may be functional in a human cell will typically not be recognized in a bacterium. A heterologous cDNA has a random but high probability of encoding a pause site somewhere, often leading to protein expression aberration failure as noted above.

Differences between pause signal coding among bacteria or among vertebrates is sufficient to make cross-family gene expression unpredictable. For example, in various organisms such as bacteria, a significant pause or translational slowing can result in premature transcription termination and/or message (or mRNA) degradation. Even in eukaryotes there is a coupling between export of mRNA from the nucleus and translation; thus a different, but still effective system of clearing untranslated mRNA exists in eukaryotes.

As provided herein, a test of translation pausing or slowing as a result of codon pair usage can be performed by comparing a series of genes that have random pauses with modified genes where codon pairs predicted to cause translational pauses are replaced by codon pairs not predicted to cause a translational pause. Unmodified genes moved from their source organism and expressed in a heterologous host can have an altered set of codon pairs predicted to cause a translational pause (e.g., an altered set of over-represented codon pairs), resulting in altered configuration of presumed pause sites. Creation of synthetic codon-pair-optimized genes can have a dramatic effect on expression: expression of difficult-to-express genes can be seen for the first time or improved at least 2-fold, 3-fold, 4-fold, 5-fold, 6-fold, 7-fold, 8-fold, 9-fold, 10-fold, 12-fold, 15-fold, 20-fold, 25-fold, 30-fold, or more, relative to unmodified polypeptide-encoding nucleic acid sequences.

Determination of Translational Kinetics Values for Codon Pairs

The methods and graphical displays provided herein include determination and use of translational kinetics values for codon pairs. As provided herein, such a translational kinetics value can be calculated and/or empirically measured, and the final translational kinetics value used in the graphical displays and methods of predicting translational kinetics and methods of designing or modifying a polypeptide-encoding nucleotide sequence provided herein can be a refined value resultant from two or more types of codon pair translational kinetics information. The various types of codon pair translational kinetics information that can be used in refining or replacing a translational kinetics value for a codon pair include, for example, values of observed versus expected codon pair frequencies in a particular organism, normalized values of observed versus expected codon pair frequencies in a particular organism, clustered observed versus expected codon pair frequencies, generic observed versus expected codon pair frequencies, the degree to which observed versus expected codon pair frequency estimated translational kinetics values are conserved in related proteins across two or more species, the degree to which estimated translational kinetics observed versus expected codon pair frequency values are conserved at predicted pause sites such as boundaries between autonomous folding units in related proteins across two or more species, the degree to which estimated translational kinetics values are conserved at predicted pause site absences such as the interior of autonomous folding units in related proteins across two or more species, the degree to which codon pairs are conserved at predicted pause sites or at predicted pause site absences across different proteins in the same species, and empirical measurement of translational kinetics for a codon pair.

The values of observed versus expected codon pair frequencies in a host organism can be determined by any of a variety of methods known in the art for statistically evaluating observed occurrences relative to expected occurrences. Regardless of the statistical method used, this typically involves obtaining codon sequence data for the organism, for example, on a gene-by-gene basis. In some embodiments, the analysis is focused only on the coding regions of the genome. Because the analysis is a statistical one, a large database is preferred. Initially, the total number of codons is determined and the number of times each of the 61 non-terminating codons appears is determined. From this information, the expected frequency of each of the 3721 (61²) possible non-terminating codon pairs is calculated, typically by multiplying together the frequencies with which each of the component codons appears. This frequency analysis can be carried out on a global basis, analyzing all of the sequences in the database together; however, it is typically done on a local basis, analyzing each sequence individually. This will tend to minimize the statistical effect of an unusually high proportion of rare codons in a sequence. After the frequency data is obtained, for each sequence in the database, the expected number of occurrences of each codon pair is calculated by, for example, multiplying the expected frequency by the number of pairs in the sequence. This information can then be added to a global table, and each next succeeding sequence can be analyzed in like manner. This analysis results in a table of expected and observed values for each of the 3271 non-terminating codon pairs. The statistical significance of the variation between the expected and observed values can then be calculated, and the resulting information can be used in further practice of the various examples and embodiments provided herein.

In some embodiments, the values of observed versus expected codon pair frequencies are chi-squared values, such as chi-squared 2 (chisq2) values or chi-squared 3 (chisq3) values. Methods for calculating chi-squared values can be performed according to any method known in the art, as exemplified in U.S. Pat. No. 5,082,767, which is incorporated by reference herein in its entirety. The result of chi-squared calculations is a list of 3,721 non-terminating codon pairs, each with an expected and observed value, together with a value for chi-squared (chisq1):
chisq1=(observed−expected)²/expected

In order to remove the contribution to chi-squared of non-randomness in amino acid pairs, a new value chi-squared 2 (chisq2) can be calculated as follows. For each group of codon pairs encoding the same amino acid pair (i.e., 400 groups), the sums of the expected and observed values are tallied; any non-randomness in amino acid pairs is reflected in the difference between these two values. Therefore, each of the expected values within the group is multiplied by the factor [sum observed/sum expected], so that the sums of the expected and observed values with the group are equal. The new chi-squared, chisq2, is evaluated using these new expected values. Calculation methods for removing the contribution to chi-squared of non-randomness in amino acid pairs are known in the art, as exemplified in Gutman .and Hatfield, Proc. Natl. Acad. Sci. USA, (1989) 86:3699-3703.

Further, in order to remove the contribution to chi-squared of non-randomness in dinucleotides, a new value chi-squared 3 (chisq3) can be calculated. Correction is made only for those dinucleotides formed between adjacent codon pairs; any bias of dinucleotides within codons (codon triplet positions I-II and II-III) will directly affect codon usage and is, therefore, automatically taken into account in the underlying calculations. For each dinucleotide pair formed between adjacent codon pairs (i.e., 16 pairs), the sums of the expected and observed values are tallied; any non-randomness in dinucleotide pairs is reflected in the difference between these two values. Therefore, each of the expected values within the group is multiplied by the factor [sum observed/sum expected], so that the sums of the expected and observed values with the group are equal. The new chi-squared, chisq3, is evaluated using these new expected values.

As provided herein, and as will be readily apparent to those skilled in the statistical art, that further values chi-squared N (chisqN) could be calculated similarly by removing one or more other variables in like fashion.

Analyses of the E. coli, S. cerevisiae, and human databases illustrate two important features. First, there is a highly significant codon pair bias in all three species, even after the amino acid nearest neighbor bias (chisq2) and the dinucleotide bias (chisq3) are discounted. Second, the effect associated with dinucleotide bias, i.e., the difference between chisq2 and chisq3, is much more pronounced in eukaryotes than in E. coli. It is by far the predominant effect in mammals, representing two thirds of the amount of chisq2 in excess of its expectation in human. Mouse and rat data exhibit a very similar pattern. Dinucleotide bias represents a smaller effect in yeast, and only a very minor one in E. coli. Although the predominant dinucleotide bias in human is the well-known CpG deficit, other dinucleotides are also very highly biased. For example, there is a deficit of TA, as well as an excess of TG, CA and CT. Overall, the deficit of CpG contributes only 35% of the total dinucleotide bias in the human database, and 17% in yeast.

In one embodiment, redundant nucleotide sequences are clustered and weighted according to the size of the cluster in the calculation of observed versus expected codon pair frequency values. Typically, databases contain redundant nucleotide sequences that are either identical or highly homologous. In such instances, consideration of all such redundant nucleotide sequences can skew the calculation of observed versus expected codon pair frequency values, where highly represented codon pairs of the redundant nucleotide sequences may be improperly calculated as over-represented in the particular organism. A standard manner for eliminating skewed observed versus expected codon pair frequency value calculation due to the presence of redundant nucleotide sequences is to select only a single sequence from these redundant sequences when performing the observed versus expected codon pair frequency value calculation. However, as provided herein, inclusion of clustered and weighted redundant nucleotide sequences can provide observed versus expected codon pair frequency values that are more statistically reliable than those provided when only a single redundant nucleotide sequence is used in the observed versus expected codon pair frequency value calculation. Thus, also contemplated herein, for all methods of making a graphical display, comparing graphical displays, modifying translational kinetics of a polypeptide-encoding nucleotide sequence, and redesign of polypeptide-encoding nucleotide sequences provided herein, as well as related sequences and graphical displays, and any other subject matter provided herein that is based, at least in part, on observed versus expected codon pair frequency values, observed versus expected codon pair frequency values calculated from clustered and weighted redundant nucleotide sequences can be used as a basis for predicting a codon-pair based translational pause.

Thus, provided herein are methods of calculating observed versus expected codon pair frequency values of codon pair frequency with improved statistically reliability, where the method comprises including clustered and weighted redundant nucleotide sequences in the calculation of observed versus expected codon pair frequency values. Redundant nucleotide sequences as used herein refers to nucleotide sequences that are either identical or highly homologous such that one skilled in the art would typically avoid including more than one such sequence in a genome-wide statistical analysis of nucleotide sequences, such as, for example, a calculation of codon usage for a particular organism. Typically, redundant nucleotide sequences are at least, or at least about, 35, 50, 60, 70, 80, 90, 91, 92, 93, 94, 95, 96, 97, 98, 99, 99.5, 99.8, or 99.9%, or more, identical to each other. As another example, redundant nucleotide sequences are those with an E value of no more than or no more than about 0.1, 0.05, 0.01, 0.005, 0.001, 0.0005, 0.0001, or 0.00005, or less, where E value is the probability of obtaining, by chance, another sequence that aligns to the query sequence with a similarity greater than the given measure of the similarity of the query sequence to the aligned target sequence; methods of calculating E values are known in the art. Determination that any two or more nucleotide sequences are redundant can be performed using any of a variety of methods known in the art, for example, BLAST. The methods provided herein comprise including clustered and weighted redundant nucleotide sequences in the calculation of observed versus expected codon pair frequency values. As used herein, clustered redundant nucleotide sequences refers to nucleotide sequences that have been determined to be redundant with one or more other nucleotide sequences in the database, where the two or more sequences that are redundant with each other are marked as belonging to the same cluster, and, thus, are clustered. As used herein, weighted redundant nucleotide sequences refers to clustered redundant nucleotide sequences whose codon pairs have been scored in a manner that reflects the size of the cluster, where the larger the size of the cluster, the codon pairs are scored such that each individual codon pair observation within the cluster contributes a lesser amount to the overall calculation of observed versus expected codon pair frequency values, thus resulting in the codon pairs within the cluster being weighted according to the size of the cluster. To illustrate, 10 redundant nucleotide sequences can be identified as belonging to a cluster, and the codon pairs of these 10 redundant nucleotide sequences can be weighted by the inverse of the size of the cluster (i.e., 1/10), such that each observation of a codon pair within the clustered redundant nucleotide sequences has 1/10 of the weight of an observed codon pair from an unclustered nucleotide sequence. The inventors have discovered that inclusion of all redundant nucleotide sequences in the calculation of observed versus expected codon pair frequency values by clustering and weighting the redundant nucleotide sequences increases the statistical reliability of the ultimate observed versus expected codon pair frequency values for codon pairs. Thus, in accordance with the methods provided herein, an improved calculation of observed versus expected codon pair frequency values can be performed by (a) clustering redundant nucleotide sequences, (b) weighting the codon pairs of the clustered nucleotide sequences according to the size of the cluster, and (c) calculating the observed versus expected codon pair frequencies of an organism using the weighted codon pairs of the clustered nucleotide sequences. For example, an improved calculation of observed versus expected codon pair frequency values can be performed by (a) clustering redundant nucleotide sequences, (b) weighting the codon pairs of the clustered nucleotide sequences by the inverse of the size of the cluster, and (c) calculating the observed versus expected codon pair frequencies of an organism using the weighted codon pairs of the clustered nucleotide sequences. Step (c) of calculating the observed versus expected codon pair frequencies of an organism can be performed in a manner consistent with the teachings provided herein for calculation of observed versus expected codon pair frequency values. Typically, all known nucleotide sequences for an organism are included in such calculation of observed versus expected codon pair frequency values.

Also provided herein are methods of calculating generic observed versus expected codon pair frequency values directed to two or more different types of organisms. It is generally recognized for statistical methods that a larger amount of data typically increases the reliability of a calculation relative to a smaller amount of data. In the context of extracting information from nucleotide sequences, an increase in the nucleotide sequence information available can yield more reliable results. For example, for some organisms, only a limited amount of nucleotide sequence data is presently available, and it may be difficult to calculate reliable values for expected versus observed codon pair frequency. Nevertheless, useful observed versus expected codon pair frequency values can be calculated by utilizing nucleotide sequence information from multiple types of organisms in calculating generic observed versus expected codon pair frequency values reflective of all combined organism types. As used herein, a generic observed versus expected codon pair frequency value refers to an observed versus expected codon pair frequency value that reflects observed versus expected codon pair frequencies of a particular codon pair for two or more different organism types. A generic observed versus expected codon pair frequency value can reflect observed versus expected codon pair frequencies for any of a wide variety of collections of organism types. For example, a generic observed versus expected codon pair frequency value can reflect observed versus expected codon pair frequencies for organisms in different orders of a class, organisms in different families of an order, organisms in different genera of a family, or organisms in different species of a genus. As another example, a generic observed versus expected codon pair frequency value can reflect observed versus expected codon pair frequencies for organisms in different subsets of a phylogenetic classification (e.g., different suborders of an order, different subclasses of a class, different subfamilies of a family, different suborders of an order, different subgenera of a genus, or different subspecies of a species). One of skill in the art can readily appreciate that the methods provided herein can be used to group any of a variety of organism types according to their relatedness, whether the relatedness is defined by traditional taxonomic nomenclature, other known classification nomenclature, or statistical determination of relatedness of organisms. Typically, the grouping of different organism types includes at least different species or different subspecies.

Methods for calculating generic observed versus expected codon pair frequency values directed to two or more different types of organisms include selecting organism types to include into the group, assembling the nucleotide sequence data available for each selected organism type, and calculating observed versus expected codon pair frequency values based on the assembled nucleotide sequence data. As provided above, the selected organism types can have any of a variety of relationships toward each other; for example, the selected organism types can be different strains or subspecies of a particular species, different species within a particular genus, different genera within a family, and the like, consistent with the teachings above. After selection of the organism types to include into the group, the nucleotide sequence data available for each organism type are assembled. The data that are assembled can be modified according to standard methods to remove or limit the nucleotide sequence data that might adversely influence the calculation of observed versus expected codon pair frequency values. For example, all but one redundant nucleotide sequence from a particular organism type can be removed. In some embodiments, some or all of the data that are assembled can be clustered and weighted according to the methods provided herein, where nucleotide sequence data from each of one or more particular organism types can have redundant nucleotide sequences clustered and weighted according to the size of the cluster, as described in more detail elsewhere herein. Calculation of observed versus expected codon pair frequency values can then be calculated for the assembled nucleotide sequence data according to any of a variety of known methods provided herein or otherwise known in the art.

As contemplated herein, organisms that are evolutionarily related are likely to share similar codon pair-based translational kinetics propensities. Accordingly, codon pair-based translational kinetics values are likely to be similar for evolutionarily related organisms. Thus, while some differences in codon pair-based translational kinetics propensities may exist for different organism types, these differences are expected to be generally smaller for organisms that are increasingly evolutionarily related, and these differences are expected to be generally greater for organisms that are increasingly evolutionarily diverged. Thus, as provided herein, nucleotide sequence data from related organism types can be grouped together in performing codon pair frequency-based translational kinetics values calculations to generate generic observed versus expected codon pair frequencies that apply to the group of related organism types. Despite the possibility of differences between the grouped organism types, the increased amount of nucleotide sequence data available for the generic chi-squared value calculation can result in generic observed versus expected codon pair frequency values that are more statistically reliable for an individual organism type than observed versus expected codon pair frequency values calculated from the nucleotide sequence data of only one organism type. While not intending to be limited by the following, it is contemplated that for a variety of particular organism types, for example, species, the shortage of available nucleotide sequence data limits the ability to accurately calculate observed versus expected codon pair frequency values for codon pairs of that organism type; by instead of calculating observed versus expected codon pair frequency values for individual organism types (e.g., individual species), observed versus expected codon pair frequency values are calculated for a group of related organism types (e.g., a group of species within the same genus), the larger amount of nucleotide sequence data can increase the statistical reliability of the calculation of observed versus expected codon pair frequency values without significantly misrepresenting observed versus expected codon pair frequency values for any particular organism type. This is particularly true when the amount of error resultant from the lack of nucleotide data is much larger than the evolutionary divergence between the grouped organism types.

In addition to the above, regardless of whether or not a large amount of nucleotide sequence data is available for one or more organism types, grouping of organism types can provide valuable information regarding observed versus expected codon pair frequency values. For example, a generic observed versus expected codon pair frequency value, by virtue of reflecting information from multiple organism types and a larger amount of data, can represent an observed versus expected codon pair frequency value that is approximately common to all grouped organism types, where the actual difference between organism types may vary by less than the increased statistical error that would result if each organism type were examined individually instead of in a group. For example, it may be that generic observed versus expected codon pair frequency values for codon pairs in primates may more be a more statistically reliable representation of actual codon pair-based translational pauses than observed versus expected codon pair frequency values calculated using only human nucleotide sequence data. Thus, also contemplated herein, for all methods of making a graphical display, comparing graphical displays, modifying translational kinetics of a polypeptide-encoding nucleotide sequence, and redesign of polypeptide-encoding nucleotide sequences provided herein, as well as related sequences and graphical displays, and any other subject matter provided herein that is based, at least in part, on observed versus expected codon pair frequency values, generic observed versus expected codon pair frequency values can be used as a basis for predicting a codon-pair based translational pause.

Generic observed versus expected codon pair frequency values also can provide a description of the commonly shared observed versus expected codon pair frequency values for various organism types of the group, and, thus, provide observed versus expected codon pair frequency values for any organism type that could be classified in the group. For example, if rat, mouse, hamster, gerbil and other rodent nucleotide sequence data were used in the calculation of observed versus expected codon pair frequency values for the order Rodentia, such values for the order Rodentia could be applied to rodents that were not included in the calculation, such as chipmunk, to provide observed versus expected codon pair frequency values by which codon pair-based translational pauses could be predicted even if very little nucleotide sequence data for other rodents such as chipmunk were available. Thus, also contemplated herein is the use of generic observed versus expected codon pair frequency values for the prediction of codon-pair based translational pauses in polypeptide-encoding nucleotide sequences from organisms for which observed versus expected codon pair frequency values have not been or cannot be accurately calculated.

Generic observed versus expected codon pair frequency values also can provide a baseline value of observed versus expected codon pair frequency values from which baseline organism type-specific deviations can be calculated. For example, if generic observed versus expected codon pair frequency values of the order Primates are calculated, species specific, for example, human-specific, deviation of observed versus expected codon pair frequency values can be calculated for instances in which the observed versus expected codon pair frequency values of the species differs from the order Primates in a statistically significant manner. As contemplated herein, generic observed versus expected codon pair frequency values, which are based on more data than observed versus expected codon pair frequency values calculated for only a single organism type (e.g., a single species), can be more statistically reliable than observed versus expected codon pair frequency values calculated for only a single organism type. Using such calculated generic observed versus expected codon pair frequency values as a baseline, nucleotide sequence data for one or more single organism types can be compared to the generic observed versus expected codon pair frequency values, and any difference that is deemed statistically significant can be applied to the generic observed versus expected codon pair frequency values and thereby generate organism type-specific observed versus expected codon pair frequency values that reflect the statistically significant difference from the generic values. A difference that is statistically significant as used in the context of the above refers to a difference in observed versus expected codon pair frequency values that is greater than the estimated errors of the observed versus expected codon pair frequency values; any of a variety of methods known in the art for evaluating the statistical significance of a difference between values can be used for such a determination. For example, any statistically significant difference between Primates observed versus expected codon pair frequency values and human observed versus expected codon pair frequency values can be applied to the Primates observed versus expected codon pair frequency values to develop refined human observed versus expected codon pair frequency values. Thus, provided herein are methods of refining observed versus expected codon pair frequency values by calculating generic observed versus expected codon pair frequency values, calculating individual organism type (e.g., species) observed versus expected codon pair frequency values, determining if and difference between the generic observed versus expected codon pair frequency values and individual organism type observed versus expected codon pair frequency values is statistically significant, and modifying the generic observed versus expected codon pair frequency values according to the statistically significant difference to arrive at refined individual organism type observed versus expected codon pair frequency values. As will be appreciated by one skilled in the art, the method of calculating generic observed versus expected codon pair frequency values and determining refined individual organism type observed versus expected codon pair frequency values based on the statistically significant difference between the values can be performed iteratively: observed versus expected codon pair frequency values can be calculated for a large group of organism types (e.g., the class Mammalia), and specific observed versus expected codon pair frequency values can be determined for different subgroups of the large group (e.g., orders Rodentia and Primates) based on statistically significant differences from the values calculated for the large group, and specific observed versus expected codon pair frequency values can be determined for different organism types (e.g., mouse and human) based on statistically significant differences from the values calculated for the subgroups.

As provided herein, the values of observed versus expected codon pair frequencies in a host organism herein can be normalized. Normalization permits different sets of values of observed versus expected codon pair frequencies to be compared by placing these values on the same numerical scale. For example, normalized codon pair frequency values can be compared between different organisms, or can be compared for different codon pair frequency value calculations within a particular organism (e.g., different calculations based on input sequence information or based on different calculations such as chisq1or chisq2 or chisq3). Typically, normalization results in codon pair frequency values that are described in terms of their mean and standard deviation from the mean.

An exemplary method for normalizing codon pair frequency values is the calculation of z scores. The z score for an item indicates how far and in what direction that item deviates from its distribution's mean, expressed in units of its distribution's standard deviation. The mathematics of the z score transformation are such that if every item in a distribution is converted to its z score, the transformed scores will have a mean of zero and a standard deviation of one. The z scores transformation can be especially useful when seeking to compare the relative standings of items from distributions with different means and/or different standard deviations. z scores are especially informative when the distribution to which they refer is normal. In a normal distribution, the distance between the mean and a given z score cuts off a fixed proportion of the total area under the curve.

An exemplary method for determining z scores for codon pair chi-squared values is as follows: First, a list of all 3721 possible non-terminating codon pairs is generated. Second, for the i^{th} codon pair, the i^{th} chi-squared value is calculated, where the i^{th} chi-squared value is denoted cᵢ. The chi-squared value, cᵢ, is given the sign of (observed−expected), so that over-represented codon pairs are assigned a positive cᵢ and under-represented codon pairs are assigned a negative cᵢ. The formula for cᵢ is:
cᵢ=sgn(obsᵢ−expᵢ)*(obsᵢ−expᵢ)²/expᵢ

Third, the mean chi-squared value is calculated where the mean is denoted m. The formula for the mean is:
m=(Sⁱcᵢ)/3721
where Sⁱ means sum over i. Fourth, the standard deviation of the chi-squared values is calculated, where the standard deviation is denoted s. The formula for the standard deviation is:
s=v(Sⁱ(cᵢ−m)²/3721)
where v means square root. Fifth, for the i^{th} chi-squared value cᵢ, a z score is calculated by subtracting the mean then dividing by the standard deviation, wherein the i^{th} z score is denoted zᵢ. The formula for the z score is:
zᵢ=(cᵢ−m)/s

The above-described values of observed codon pair frequency versus expected codon pair frequency can be used as first approximations of translational kinetics of a polypeptide-encoding nucleotide sequence. However, such values do not always accurately predictor translational kinetics, and refinement of such values to more accurately predict translational kinetics can be performed according to the methods provided herein. Thus, among the methods provided herein are methods of refining the predictive capability of a translational kinetics value of a codon pair in a host organism by providing an initial translational kinetics value based on the value of observed codon pair frequency versus expected codon pair frequency for a codon pair in a host organism, providing additional translational kinetics data for the codon pair in the host organism, and modifying the initial translational kinetics value according to the additional codon pair translational kinetics data to generate a refined translational kinetics value for the codon pair in the host organism. The translational kinetics data that can be used to refine translational kinetics values and methods of modifying translational kinetics values according to such additional translational kinetics data to generate a refined translational kinetics value for a codon pair in a host organism are provided below.

In one embodiment, translational kinetics data that can be used to refine translational kinetics values are based on recurrence of a codon pair and/or recurrence of a predicted translational kinetics value associated with a codon pair. Recurrence-based refinement of translational kinetics values is based on the investigation of multiple polypeptide-encoding nucleotide sequences to determine whether or not there are multiple occurrences of either codon pairs or predicted translational kinetics values in those sequences. Recurrence-based refinement of translational kinetics can be performed using any of a variety of known sequence comparison methods consistent with the examples provided herein. For purposes of exemplification, and not for limitation, the following example of recurrence-based refinement of translational kinetics is provided.

The methods provided herein relate to comparing a variety of different sources of translational kinetics information with each other in order to generate and refine translational kinetics values of codon pairs. For example, the methods provided herein can be used to compare statistically-based translational kinetics information of codon pair frequencies with translational kinetics information based on other sources such as protein relatedness, protein structure location, phylogentic relationship, empirical measurements, and other such sources of translational kinetics information provided herein or otherwise known in the art.

In some embodiments, method can be used for correlating codon pair usage in an organism with translational kinetic values, by providing a set of locations of interest in a plurality of native polypeptide-encoding nucleotide sequences, wherein the locations of interest are potentially associated with altered translational kinetics, analyzing and comparing actual codon pair utilization in the locations of interest, identifying a pattern of non-random codon pair utilization in at least some locations of interest, and correlating the non-random codon pair utilization with translational kinetic values at said at least some locations of interest. For example, a plurality of polypeptides in a plurality of organisms can be encoded by the plurality of polynucleotides, wherein the proteins are related proteins from organism to organism, and the locations of interest encode corresponding protein locations from organism to organism. In another example, a plurality of polypeptides in a plurality of organisms are encoded by the plurality of polypeptide-encoding nucleotide sequences, wherein the polypeptides are related from organism to organism, and the locations of interest encode corresponding polypeptide locations from organism to organism. In some embodiments, the polypeptide-encoding nucleotide sequences encode a plurality of different polypeptides of a particular target organism. For example, the locations of interest can be locations having an increased likelihood of being translational pause regions due to structure of the encoded polypeptides. In some embodiments, the plurality of different polypeptides can be highly expressed in the target organism, while in other embodiments, the non-random codon pair utilization is analyzed or identified by an expectation-maximization algorithm. In some embodiments, the locations of interest are provided by statistical analysis of actual versus expected codon pair usage to putatively associate particular codon pairs with translational pauses, and in which the identifying and correlating steps comprise confirming or increasing the association with translational pauses of some such codon pairs and eliminating or reducing the association with translational pauses of other such codon pairs.

Also provided are methods for correlating codon pair usage in a target organism with translational kinetics, by ascertaining statistical codon pair usage of the target organism and a plurality of other organisms, identifying a polypeptide expressed in the target organism having one or more putative translational pause sites, wherein an analogous polypeptide is expressed in the plurality of other organisms, relating actual codon pair usage at locations of polynucleotide encoding the putative translational pause sites in the target organism and corresponding locations in polynucleotide encoding the analogous polypeptides of the plurality of other organisms to statistically expected codon pair usage in each organism, and thereby correlating codon pair usage in the target organism with translational kinetics. For example, the relating step involves determining whether a putative pause site is likely to be an actual pause site. In another example, the correlating step involves determining whether a codon pair is both statistically overrepresented in codon pair usage of the target organism, and also present at putative pause sites determined likely to be actual pause sites in the relating step. In another example, the relating step comprises creating a pause conservation map showing conservation of statistically overrepresented codon pairs encoding corresponding locations in corresponding proteins in a plurality of organisms.

Similarly, also provided herein are methods of improving the predictive capability of translational kinetics values of codon pairs by providing translational kinetics values of codon pairs, and extracting translational kinetics information other than observed versus expected codon pair usage information from a plurality of polypeptide-encoding nucleotide sequences and comparing said translational kinetics information to said translational kinetics values, wherein said translational kinetics values are modified according to said translational kinetics information to generate translational kinetics values with improve the predictive capability. In some such methods, the translational kinetics information can be any of (i) translational kinetics similarities based on amino acid sequence relatedness of the encoded polypeptides, (ii) translational kinetics relationship based on phylogenetic relationship of the encoded polypeptides, (iii) presence or absence of translational pauses based on the level of expression of the polypeptides, (iv) translational kinetics similarities secondary or tertiary structural relatedness of the polypeptides, (v) translational kinetics value propensities based on a codon pair being within or outside of an autonomous folding unit of a polypeptide, (vi) empirically measured translational step times, and (vii) combinations thereof. In some such methods, the comparing step further comprises predicting said translational kinetics information based on the translational kinetics values, and said translational kinetics values are modified to improve the prediction of said translational kinetics information based on the modified translational kinetics values.

Based on the teachings provided above, one skilled in the art will recognize that such methods can be implemented by known methods. In one embodiment, expectation maximization methods can be used. To the extent that such methods cannot be implemented with known methods, below is provided guidance for the implementation. The below guidance is provided to specifically teach the methods provided herein, and is not intended to limit the scope of such methods.

Ontology

There exist stochastic regulatory events. For each species, each non-terminating codon pair or dicodon, dᵢ, has an associated regulatory probability, pᵢ, that a regulatory event will occur at a translation traversal of that dicodon. The goal is to associate each dᵢ with a corresponding pᵢ.

The basic unit of analysis is the gene multiple sequence alignment (MSA). An MSA is a matrix that consists of a set of aligned gene sequences. An MSA row corresponds to a sequence with interspersed alignment gaps. An MSA column corresponds to an aligned position within the sequences. An MSA might contain only one sequence (row). There are several MSAS, one for each aligned set of genes under analysis.

The result of analysis is a set of dicodon probability tables, one table for each species under analysis. A dicodon probability table associates each dicodon dᵢ with a corresponding probability pᵢ.

A sequence region or window is a contiguous block of columns contained within an MSA. We suppose a window is m×n i.e., species are numbered 1 to m and columns 1 to n. In one embodiment the alignment is based on sequence similarity and windows are chosen based on the label quality measures shown below. In another embodiment the alignment is based on protein structural similarity and windows are chosen based on protein structural domain boundaries and interiors.

A construct can be set where there are three mutually exclusive and exhaustive classes of windows:
(1) a conserved site is a window within which for each species at least one dicodon of that species within the window has a high probability of a regulatory event;
(2) a conserved absence is a window within which for each species no dicodon of that species within the window has a high probability of a regulatory event; and
(3) a don't-care is a window with no preferences.

Each MSA is divided into mutually exclusive and exhaustive windows and each window is labeled with exactly one of the three class labels. The null hypothesis, indicating no effect, is don't-care. For simplicity the analysis below assumes no alignment gaps in conserved site or absence windows, but this condition is not required. A window may eventually be Gaussian weighted, as will be understood to one skilled in the art, but for now for simplicity is just a simple unweighted window. A column may eventually be entropy-weighted, as will be understood to one skilled in the art, but for now for simplicity is unweighted.

For each species, each codon cᵢ has an associated codon usage probability uᵢ. For each species uᵢ is calculated from a statistical analysis of the coding regions of the species' genomic sequence by dividing the number of occurrences of cᵢby the number of occurrences of any codon encoding the amino acid encoded by cᵢ. Thus, uᵢ is the conditional probability that Cᵢ will occur given the amino acid encoded.

Expectation-Maximization (EM)

The EM process repeatedly iterates two steps. In the first step we assume the dicodon probabilities pᵢ are correct and use them to assign labels to sequence windows based on comparison to the null hypothesis. In the second step we adjust pᵢ by gradient descent to maximize the likelihood of the sequence labels from the first step. This two-step process iterates many times.

First Step: Assigning Labels to Sequence Windows

Given a candidate window within an MSA and a candidate label (site or absence) as a hypothesis for the window, the label hypothesis competes with the null hypothesis (don't-care) to see which best explains the data. Here the data are the observed dicodons within the window, with the observed amino acid sequence as an underlying assumed constraint.

The null hypothesis is set such that the codons in the window were selected randomly based on the species' known codon usage frequencies given the observed amino acid sequence. This immediately yields the probability of the observed dicodons in the window, as the product of the respective codon usage probabilities uᵢ.

The label hypothesis is similar, but computes a conditional probability from the codon usage frequencies uᵢ where the condition is that the criteria associated with the label is satisfied by the observed dicodons. To make this practical, we compute a label quality measure for the window as described below, then impose the condition that the observed codons yield a label quality measure that is equal or better. This is slightly biased because the label quality measure is computed from observed data, but that slight bias is acceptable in order to achieve a practical method.

Formally, let Hₒ be the null hypothesis and H be the label hypothesis. Let D be the observed data, i.e., the observed dicodons in the window. Then
P(Hₒ/D)=P(D/H₀)P(H₀)/P(D)
P(H/D)=P(D/H)P(H)/P(D)
where P(Hₒ) is the product of the codon usage probabilities in the window and P(Hₒ), P(H) are set as estimates from guesses about how we think protein structure is likely to behave; e.g., perhaps 2% of columns are conserved sites, 25% conserved absences, and the rest don't-cares.

To compute P(D/Hₒ) we multiply the codon usage probabilities in the window. Let u_{rc} be the codon usage probability at row (species) r and column (codon) c in the MSA under the window. Then

$$P ( D / {H}_{o} ) = {\prod }_{i = 1}^{m} {\prod }_{j = 1}^{n} {u}_{ij}$$

To compute P(D/H) we require a label quality measure Q and a way to estimate the probability P_{Q} that a randomly drawn sequence, weighted by codon usage probabilities uᵢ is as good as or better than Q. Then P(D/H)=P(D/H_{O})/P_{Q} and the odds ratio of the label hypothesis to the null hypothesis is 1/P_{Q}.

Label Quality Measure Q

Let p_{rc} be the dicodon probability at row (species) r and column (dicodon) c in the window. Suppose the window is m×n, i.e., species are numbered 1 to m and columns 1 to n. Note that n codons implies n −1 dicodons. By convention,

p_{rc} is for the dicodon at [(r, c−1), (r, c)] and pᵣ₁.

Q_{absence} is the probability that no regulatory event occurs at any dicodon in any species.

$${Q}_{absence} = {\prod }_{i = 1}^{m} {\prod }_{j = 1}^{n} ( 1 - {p}_{ij} )$$

Qₛᵢₜₑ is the probability that at least one regulatory event occurs at some dicodon in every species.

$${Q}_{site} = {\prod }_{i = 1}^{m} {\sum }_{j = 1}^{n} {p}_{ij} {\prod }_{k = 1}^{j - 1} ( 1 - {p}_{ik} ) = {\prod }_{i = 1}^{m} ( 1 - {\prod }_{j = 1}^{n} ( 1 - {p}_{ij} ) )$$

For error tolerance it is convenient to define Q* analogous to Q except that the most egregious error is within this window removed.

$${Q}_{absence}^{*} = {Q}_{absence} except set {p}_{ij} = 0 in the term corresponding to$$

$${min}_{i , j} ( 1 - {p}_{ij} )$$

$${Q}_{site}^{*} = {Q}_{site} except set {p}_{ij} = 1 in the term corresponding to$$

$${max}_{i} {min}_{j} ( 1 - {p}_{ij} )$$

For assigning labels to windows use Q*. For defining a gradient use Q.

Alternatively, and more easily, Q_{absence} and Qₛᵢₜₑ can be obtained from simple recursions.

$${R}_{i l} = 1$$

$${R}_{ij} = {\prod }_{k = 1}^{j} ( 1 - {p}_{ik} ) = ( 1 - {p}_{ij} ) {R}_{i ( j - 1 )}$$

$${Q}_{absence} = {\prod }_{i = 1}^{m} {R}_{i n}$$

$${Q}_{site} = {\prod }_{i = 1}^{m} ( 1 - {R}_{i n} )$$

Estimating P_{Q}

Recall that two different kinds of probabilities are under discussion:

(1) the probability of the occurrence or absence of a regulatory event, estimated by the dynamically changing dicodon probabilities pᵢ which are the object of EM;

(2) the probability of observing a specific sequence of dicodons, or a set of sequences of dicodons satisfying some quality measure as described above, estimated from static genomic codon usage frequencies u_{c}, where u_{c} is the codon usage conditional probability of codon c given the species and the amino acid encoded by c.

Here we assume a quality measure, Q (either Qₛᵢₜₑ or Q_{absence}), already has been computed for the window, as above. Assuming random codons are drawn for each amino acid in the window according to codon usage frequencies, we estimate the probability P_{Q} that the resulting random dicodons will equal or better the quality measure Q.

This is done by estimating the distribution of Rᵢⱼ empirically. The distribution of Rᵢⱼ is estimated by combining estimates of the distributions of Rᵢⱼₖ, where Rᵢⱼₖ is the component of Rᵢⱼ that ends in codon k. This done, the empirical distribution for P_{Q} can be obtained by combining the distributions of Rᵢₙ for each row as shown below.

The data structure ERD (Empirical R Distribution) is:

(1) A=array holding the distribution, assumed zero-based.

(2) B=current maximum possible value of Rᵢⱼ.

(3) U=associated codon usage probability mass total.

The array A size is a, say a=1000. A[i] represents the codon usage probability mass associated with the event probability half-open interval [x, x+δ) where

δ=B/a

x=i*δ, assuming A is zero-based

A[i] is assumed uniformly distributed over [x, x+δ).

One ERD is created for each possible codon of each amino acid in the window. ERDᵢⱼₖ corresponds to row i, column j, codon k. Values are propagated left-to right for each row i, then combined as shown below to yield an ERD corresponding to Rᵢₙ. Finally, P_{Q} is obtained from ERDs for Rᵢₙ as shown below.

Rᵢⱼₖ holds the probability distribution for Rᵢⱼ with k being the last codon. An ERD for Rᵢⱼ can be produced from the ERDs for Rᵢⱼₖ using the procedure shown by artificially constraining the amino acid at i(j+1) to have only a single codon with uₗ=1. Then the ERD for Rᵢⱼ is equal to the ERD for Rᵢ₍ⱼ₊₁₎ₗ that results. In practice, it is only necessary to produce ERDs for Rᵢₙ.

Initialization for Rᵢⱼₖ

Initialization at (j=1)
Aᵢⱼₖ[h]=0, 0≦h<a−1
Aᵢⱼₖ[a−1]=1
Bᵢⱼₖ=1
Uᵢⱼₖ=uᵢⱼₖ
where uᵢⱼₖ is the codon usage probability of the k^{th} codon of the amino acid at (i, j). Initialization at (j>1), after ERDs for Rᵢ₍ⱼ₋₁₎ are complete

$$\begin{matrix}{A}_{ijk} [ h ] = 0 , 0 \leq h < a \\ {B}_{ijk} = {max ( 1 - {p}_{xk} ) {B}_{i ( j - 1 ) x}}_{x} \\ {U}_{ijk} = 0\end{matrix}$$

where x is a possible codon of the amino acid at (i, j−1) and Pₓₖ is the regulatory probability of the dicodon (x, k).
Recursion for Rᵢⱼₖ

Recursion methods can be conducted according to:

```
          FOR (x a possible codon of the amino acid at (i, j − 1)) DO
    Uᵢⱼₖ = Uᵢⱼₖ + uₖUᵢ₍ⱼ₋₁₎ₓ
    δₓ = Bᵢ₍ⱼ₋₁₎ₓ / a
    δₖ = Bᵢⱼₖ / a
    FOR h FROM 0 TO (a − 1) DO
        r = hδₓ
        s = r + δₓ
        y = r / δₖ
        z = s / δₖ
        e = └y┘
        f = └z┘
        IF (e = f) THEN
            Aᵢⱼₖ[e] = Aᵢⱼₖ[e] + uₖAᵢ₍ⱼ₋₁₎ₓ[h]
        ELSE
            w = (e + 1 − y)/(z − y)
            Aᵢⱼₖ[e] = Aᵢⱼₖ[e] + wuₖAᵢ₍ⱼ₋₁₎ₓ[h]
            w = (z − f)/(z − y)
            Aᵢⱼₖ[f] = Aᵢⱼₖ[f] + wuₖAᵢ₍ⱼ₋₁₎ₓ[h]
            FOR g FROM(e + 1) TO (f − 1) DO
                w = 1/(z − y)
                Aᵢⱼₖ[g] = Aᵢⱼₖ[g] + wuₖAᵢ₍ⱼ₋₁₎ₓ[h]
            ENDDO
        ENDIF
    ENDDO
ENDDO
```

Combining to Yield P_{Q}

To compute a probability distribution for Rᵢₙ we convolve the ERPs for Rᵢₙₖ. To compute a probability distribution for Q_{absence} we convolve the ERPs corresponding to Rᵢₙ. To compute a probability distribution for Qₛᵢₜₑ we convolve ERPs corresponding to (1−Rᵢₙ).

Convolving ERPs

Convolving ERDs corresponds to multiplying the underlying probabilities. Suppose X₁, . . . , Xₘ are ERPs that we wish to convolve. The result will be placed in the ERP Y. The ERP Z is used to hold intermediate calculations. Initialize by copying X₁ into Y.

```
FOR i FROM 2 TO m DO
    Copy Y into Z.
    B_{Y} = B_{z}B_{Xi}
    U_{Y} = U_{z}U_{Xi}
    A_{Y}[k] = 0, 0 ≦ k < a
    δ_{X} = B_{Xi} / a
    δ_{Y} = B_{Y} / a
    δ_{Z} = B_{Z} / a
    FOR j FROM 0 TO (a − 1) DO
        FOR k FROM 0 TO (a − 1) DO
            r₁ = jδ_{X}
            r₂ = kδ_{Z}
            s₁ = r₁ + δ_{X}
            s₂ = r₂ + δ_{X}
            y = r₁r₂ / δ_{Y}
            z = s₁s₂ / δ_{Y}
            e = └y┘
            f = └z┘
            p = A_{Xi}[j]A_{Z}[k]
            IF (e = f) THEN
                A_{Y}[e] = A_{Y}[e]+ p
            ELSE
                w = (e + 1 − y)/(z − y)
                A_{Y}[e] = A_{Y}[e]+ wp
                w = (z − f)/(z − y)
                A_{Y}[f] = A_{Y}[f]+ wp
                FOR g FROM(e + 1) TO (f − 1) DO
                    w = 1/(z − y)
                    A_{Y}[g] = A_{Y}[g]+ wp
                ENDDO
            ENDIF
        ENDDO
    ENDDO
ENDDO
Y holds the convolution of X₁,...,Xₘ.
```

Computing (1−ERP)

Suppose X is an ERP and we desire an ERP for (1−X). The result will be placed in the ERP Y.

```
    U_{Y} = U_{X}
    δ_{X} = B_{X} / a
    L = 0
    WHILE (A_{X}[L] = 0) DO L = L + 1 ENDDO
    B_{Y} = 1 − Lδ_{X}
    δ_{Y} = B_{Y} / a
    FOR k FROM L TO (a − 1) DO
        r = kδₓ
        s = r + δₓ
        y = (1 − s) / δ_{y}
        z = (1 − r) / δ_{y}
        e = └y┘
        f = └z┘
        p = A_{X}[k]
        IF (e = f) THEN
            A_{Y}[e] = A_{Y}[e]+ p
        ELSE
            w = (e + 1 − y)/(z − y)
            A_{Y}[e] = A_{Y}[e]+ wp
            w = (z − f)/(z − y)
            A_{Y}[f] = A_{Y}[f]+ wp
            FOR g FROM(e + 1) TO (f − 1) DO
                w = 1/(z − y)
                A_{Y}[g] = A_{Y}[g]+ wp
            ENDDO
        ENDIF
    ENDDO
Y holds (1 − X).
```

Computing P_{Qabsence}

For each i, convolve ERPᵢₙₖ across k, leaving the result in ERPᵢₙ.

Convolve ERP₁ₙ, . . . , ERPₘₙ, leaving the result in ERPY. Note that

```
Q_{absence} ≦ B_{Y} .
    P_{Qabsence} = 0
    δ_{Y} = B_{Y} / a
q = Q_{absence} / δ_{Y}
e = └q┘
FOR k FROM 0 TO e DO
    P_{Qabsence} = P_{Qabsence} + A_{Y}[k]
ENDDO
w = (q − e)
P_{Qabsence} = P_{Qabsence} + wA_{Y}[e + 1]
P_{Qabsence} = U_{Y}P_{Qabsence}
```

Computing P_{Qsite}

Compute X₁, . . . , Xₘ as (1−ERP₁ₙ), . . . , (1−ERPₘₙ). Then follow the instructions in “Computing Q_{absence}” substituting “Xᵢ” for “ERPᵢₙ₁” and substituting “P_{Qsite}” for “P_{Qabsence.”}

Second Step: Adjusting pᵢ

The dicodon probabilities pᵢ are updated by standard gradient descent.

For each window, the gradients are:

$$\frac{\partial {Q}_{absence}}{\partial {p}_{ij}} = - {\prod }_{( {i}^{′} , {j}^{′} ) \neq ( i , j )} ( 1 - {p}_{{i}^{′} {j}^{′}} )$$

$$\begin{matrix}\frac{\partial {Q}_{site}}{\partial {p}_{ij}} = [ {\prod }_{{i}^{′} \neq i} {\sum }_{{j}^{′} = 1}^{n} {p}_{{i}^{′} {j}^{′}} {\prod }_{{k}^{′} = 1}^{{j}^{′} - 1} ( 1 - {p}_{{i}^{′} {k}^{′}} ) ] [ \begin{matrix}( {\prod }_{{k}^{′} = 1}^{j - 1} ( 1 - {p}_{{ik}^{′}} ) ) - \\ {\sum }_{{j}^{′} = j + 1}^{n} {p}_{{ij}^{′}} {\prod }_{{{k}^{′} = 1}_{{k}^{′} \neq j}}^{{j}^{′}} ( 1 - {p}_{{ik}^{′}} )\end{matrix} ] \\ = [ {\prod }_{{i}^{′} \neq i} ( 1 - {\prod }_{{j}^{′} = 1}^{n} ( 1 - {p}_{{i}^{′} {j}^{′}} ) ) ] [ {\prod }_{{j}^{′} \neq j} ( 1 - {p}_{{ij}^{′}} ) ]\end{matrix}$$

$$\frac{\partial {Q}_{absence}}{\partial {p}_{ij}} = \frac{- {Q}_{absence}}{( 1 - {p}_{ij} )}$$

$$\frac{\partial {Q}_{site}}{\partial {p}_{ij}} = [ {\prod }_{{i}^{′} \neq i} ( 1 - {R}_{{i}^{′} n} ) ] [ {R}_{i n} / ( 1 - {p}_{ij} ) ]$$

The window gradients are combined into an overall gradient. Where X is site or absence, Y is the set of windows, and w_{y} is a weight for window y,

$$\frac{\partial {Q}_{X}}{\partial {p}_{ij}} = {\sum }_{y ∈ Y} {w}_{y} \frac{\partial {Q}_{X , y}}{\partial {p}_{ij}}$$

Where α weights the relative importance of site and absence and 0<α<1,

$$\frac{\partial Q}{\partial {p}_{ij}} = α \frac{\partial {Q}_{absence}}{\partial {p}_{ij}} + ( 1 - α ) \frac{\partial {Q}_{site}}{\partial {p}_{ij}}$$

Then where δ is a small increment,

$${p}_{ij ( new )} = {p}_{ij ( old )} + \frac{\partial Q}{\partial {p}_{ij}} δ$$

In one exemplary embodiment, the predicted translational kinetics value for a codon pair can be refined according to the degree to which observed versus expected codon pair frequency values are conserved in related proteins across two or more species. As provided herein, “related proteins” refers to proteins having similar amino acid sequences and/or three dimensional structures. Related proteins having similar amino acid sequences will typically have at least about 50%, 55%, 60%, 65%, 70%, 75%, 80%, 85%, 90%, 95% sequence identity. Related proteins having similar three dimensional structures will typically share similar secondary structure topology and similar relative positioning of secondary structural elements; exemplary related proteins having three dimensional structures are members of the same SCOP-classified Family (see, e.g., Murzin A. G., Brenner S. E., Hubbard T., Chothia C. (1995). SCOP: a structural classification of proteins database for the investigation of sequences and structures. J. Mol. Biol. 247, 536-540.).

The observed versus expected codon pair frequency values for any given codon pair can vary from species to species. However, as provided herein, evolutionarily related proteins in different species will typically conserve some or all translational pause or slowing sites. Based on this, an observed conservation of one or more predicted translational pause or slowing sites in evolutionarily related proteins of different species can confirm or increase the likelihood that a translational pause or slowing site is a functional translational kinetics signal (e.g., is a functional translational pause). The codon pair located at the position on a protein that is confirmed as, or considered to have an increased likelihood of, containing an actual translational pause or slowing can itself be confirmed as being, or considered to have an increased likelihood of being, a functional translational kinetics signal (e.g., a functional translational pause). Similarly, a codon pair located at a position on a protein that is confirmed as not containing, or considered to have a decreased likelihood of containing, an actual translational pause or slowing, can itself be confirmed as not acting, or considered to have an decreased likelihood of acting, as a functional translational kinetics signal (e.g., a functional translational pause). Accordingly, initially predicted translational kinetics data, e.g., data based on values of observed codon pair frequency versus expected codon pair frequency, can be modified according to conserved codon pair frequency values across two or more species, which can lead to the codon pair being confirmed as: being a functional translational kinetics signal (e.g., a functional translational pause); being considered to have an increased likelihood of being a functional translational kinetics signal (e.g., a functional translational pause); being confirmed as not acting as an functional translational kinetics signal (e.g., a functional translational pause); or being considered to have a decreased likelihood of being a functional translational kinetics signal (e.g., a functional translational pause).

In another embodiment, the predicted translational kinetics value for a codon pair can be refined according to the presence of the codon pair at a location predicted by methods other than codon pair frequency methods to contain a translational pause or slowing site. One example of such a predicted location is a boundary location between autonomous folding units of a protein. While not intending to be limited to the following, it is proposed that translational pauses are present in wild type genes in order to slow translation of a nascent polypeptide subsequent to translation of a secondary structural element of a protein and/or a protein domain, thus providing time for acquisition of secondary and at least partial tertiary structure by the nascent protein prior to further downstream translation, and thereby allowing each domain to partially organize and commit to a particular, independent fold. As such, it is proposed herein that codon pairs can be associated with translational pauses between autonomous folding units of a protein, where autonomous folding units can be secondary structural elements such as an alpha helix, or can be tertiary structural elements such as a protein domain. Thus, the presence of a codon pair at a boundary location between autonomous folding units of a protein can confirm or increase the likelihood that the codon pair acts to pause or slow translation. Accordingly, predicted translational kinetics data, e.g., data based on values of observed codon pair frequency versus expected codon pair frequency, can be modified according to the presence of the codon pair at a boundary location between autonomous folding units of a protein, which can increase the likelihood of the codon pair acts to pause or slow translation. For example, an over represented codon pair that is present at a boundary location between autonomous folding units of a protein can be confirmed as acting as a translational pause or slowing codon pair.

In the above embodiment, a single observation of the codon pair at a boundary location between autonomous folding units of a protein can confirm or increase the likely translational pause or slowing properties of a codon pair. However, typically a plurality of observations will be used to more accurately estimate the translational pause or slowing properties of a codon pair. Thus, methods of using, for example, predicted boundary locations can be combined with methods that are based on recurrence of a codon pair and/or recurrence of a predicted translational kinetics value associated with a codon pair in methods of refining a predicted translational kinetics value for a codon pair. For example, a protein present in two or more species can have conserved boundary locations between autonomous folding units of the protein, and recurrent presence of an over-represented codon pair at the boundary locations can confirm the likelihood of an actual translational pause at that boundary location, leading to confirmation, or increased likelihood, that the corresponding codon pair for the respective species acts as a translational pause or slowing codon pair. In another example, two or more proteins of the same species can have boundary locations between autonomous folding units, and recurrent presence of an over-represented codon pair at the boundary locations can confirm or indicate the likelihood of an actual translational pause at that boundary location, leading to confirmation or indication of increased likelihood that the corresponding codon pair acts as a translational pause or slowing codon pair.

Such recurrence-based methods also can be used to confirm or indicate increased likelihood that a non-over-represented codon pair (e.g., an under-represented codon pair or a represented-as-expected codon pair) acts as a translational pause or slowing codon pair. For example, two or more proteins of the same species can have boundary locations between autonomous folding units, and recurrent presence of a non-over-represented codon pair at the boundary locations, particularly if no over-represented codon pair is present, can confirm or indicate the likelihood of an actual translational pause at that boundary location, leading to confirmation or indication of increased likelihood that the corresponding codon pair acts as a translational pause or slowing codon pair.

Such recurrence-based methods also can be used to confirm or indicate the likelihood that a codon pair, such as an over-represented codon pair, does not act as a translational pause or slowing codon pair. For example, two or more proteins of the same species can have boundary locations between autonomous folding units, and consistent absence of a non-over-represented codon pair at the boundary locations, can confirm or indicate the increased likelihood that the codon pair does not act as a translational pause or slowing codon pair.

In another embodiment, presence of a codon pair in a highly expressed protein can confirm or increase the likelihood that the codon pair does not act as translational pause or slowing codon pair. It is contemplated herein that for at least some proteins, high expression levels are reflective of an absence of translational pauses in the polypeptide encoding nucleotide sequence. Accordingly, codon pairs over-represented or always present in highly expressed proteins can be considered to be less likely to cause a translational pause or slowing relative to codon pairs under-represented or never present in highly expressed proteins. Thus, methods provided herein for refinement of translational kinetics values can include determining codon pairs over-represented or always present in one or more highly expressed proteins in an organism, and modifying the translational kinetics value of such determined codon pairs to indicate that such determined codon pairs are not likely to cause a translational pause or modifying the translational kinetics value of such determined codon pairs to decrease the likelihood that such determined codon pairs cause a translational pause. Similarly, methods provided herein for refinement of translational kinetics values can include determining codon pairs under-represented or never present in one or more highly expressed proteins in an organism, and modifying the translational kinetics value of such determined codon pairs to indicate that such determined codon pairs are likely to cause a translational pause or modifying the translational kinetics value of such determined codon pairs to increase the likelihood that such determined codon pairs cause a translational pause.

In another embodiment, the predicted translational kinetics value for a codon pair can be refined according to empirical measurement of translational kinetics for a codon pair. The influence of a codon pair on translational kinetics can be experimentally measured, and these experimental measurements can be used to refine or replace the predicted translational kinetics values for a codon pair. Several methods of experimentally measuring the translational kinetics of a codon pair are known in the art, and can be used herein, as exemplified in Irwin et al, J. Biol. Chem., (1995) 270:22801. One such exemplary assay is based on the observation that a ribosome pausing at a site near the beginning of an mRNA coding sequence can inhibit translation initiation by physically interfering with the attachment of a new ribosome to the message, and, thus, the codon pair to be assayed can be placed at or near the beginning of a polypeptide-encoding nucleotide sequence and the effect of the codon pair on translational initiation can be measured as an indication of the ability of the codon pair to cause a translational pause. Another such exemplary assay is based on the fact that the transit time of a ribosome through the leader polypeptide coding region of the leader RNA of the trp operon sets the basal level of transcription through the trp attenuator, and, thus, the codon pair to be assayed can be placed into a trpLep leader polypeptide codon region, and level of expression can be inversely indicative of the translational pause properties of the codon pair, due to a faster translation causing formation of a stemp-loop attenuator in the leader RNA, which results in transcriptional attenuation.

In one exemplary method, a gene, such as the lacZ gene from Escherichia coli can be modified such that the original protein sequence (β-galactosidase) is still encoded, but the nucleotide sequence has been modified to contain no predicted translational pauses. Codon pairs whose translational step times are to be measured can then be placed at any portion of this sequence. Since placement of codon pairs whose translational step times are to be measured can cause an amino acid change from the original protein sequence, typically the codon pairs are placed at a sequence position that does not result in an amino acid change that would alter native enzyme activity. For example, codon pairs whose translational step times are to be measured can be placed near the amino terminus such that any translational pausing caused by the codon pair is most pronounced. Typically, codon pairs whose translational step times are to be measured are placed within or within about 20, 18, 16, 14, 12, 10, 9, 8, 7, 6, 5, 4, 3, 2 or 1 amino acid of the amino terminus. For example, codon pairs whose translational step times are to be measured can be placed at amino acid positions 3 and 4, where amino acid position 1 is the amino terminal amino acid. The protein can then be expressed under conditions in which protein levels reflect the speed of translation of the protein-encoding mRNA. For example, the protein can be expressed in a cell growing in logarithmic phase that expresses steady state levels of the mRNA under examination and steady state levels of the encoded protein, where the ratio of these steady state levels reflects the speed of translation of the protein-encoding mRNA. Since the mRNA under examination has been modified to have all predicted translational pauses removed except possibly the codon pair that is added, any reduction in the ratio of translated protein to mRNA will reflect a slower translational step time caused by the added codon pair. Thus, translational step times can be empirically measured by adding a codon pair to be studied to a polypeptide encoding nucleotide sequence that does not contain any translational pauses, translating the codon pair-added polypeptide-encoding nucleotide sequence, and comparing the ratio of translated protein to mRNA of the codon pair-added polypeptide-encoding nucleotide sequence to the ratio of translated protein to mRNA of the polypeptide-encoding nucleotide sequence containing no translational pauses, where a decrease in the ratio of translated protein to mRNA of the codon pair-added polypeptide-encoding nucleotide sequence relative to the ratio of translated protein to mRNA of the polypeptide-encoding nucleotide sequence containing no translational pauses is indicative of an increased translational step time caused by the codon pair, and is indicative that the codon pair causes a translational pause. Methods of measuring levels of protein and mRNA are known in the art, and any of a variety of methods can be used in the methods provided herein. In one example, when the translated protein is an enzyme, an enzymatic assay can be performed. For example, an o-nitrophenylgalactoside-based colorometric assay as known in the art can be performed to determine the level of β-galactosidase that has been translated. Levels of mRNA can be performed by any of a variety of real time-PCR methods known in the art for quantitating mRNA levels.

In some embodiments, control experiments can be performed to confirm that the measurement of protein level is not resultant from the change in the amino acid sequence due to insertion of the codon pair to be examined. In such embodiments, each of multiple codon pairs encoding the same amino acids as the codon pair to be examined can be separately inserted into the polypeptide-encoding nucleotide sequence at the same location as the insertion site for the codon pair to be examined, and corresponding protein and mRNA levels for these codon pair-inserted polypeptide-encoding nucleotide sequences can be compared to both the translated protein and mRNA levels of the codon pair-to-be-examined inserted polypeptide-encoding nucleotide sequence and the translated protein and mRNA levels of the non-inserted polypeptide-encoding nucleotide sequence. Polypeptide-encoding nucleotide sequences that do not contain any translational pauses are expected to typically yield similar ratios of translated protein levels to mRNA levels, unless an amino acid change due to codon pair insertion modulates the measurement of translated protein levels. Such controls, and multiple measurements of the various protein and mRNA levels can be collected to generate sufficiently accurate ratios of translated protein levels to mRNA levels that permit determination by well known methods in the art of whether or not the difference between the ratio of translated protein levels to mRNA levels in the polypeptide-encoding nucleotide sequence containing the codon pair to be examined and the non-inserted polypeptide-encoding nucleotide sequence is statistically significant, and thereby reflective of a difference in translational step times, and indicative that the codon pair to be examined causes a translational pause. Such well known methods also can be used to calculate the degree of the translational step time for a particular codon pair, and to also calculate the magnitude of the translational pause caused by the codon pair.

In some embodiments, translational step time measurement methods of the polypeptide-encoding nucleotide sequence can utilize cell-free in vitro translation assays known in the art. In other embodiments, translational step time measurement methods of the polypeptide-encoding nucleotide sequence can utilize cell systems. In methods that utilize cell systems, typically cells for which gene expression has been well characterized will be used; such cells include, but are not limited to, Escherichia coli, Saccharomyces cerevisiae, Pichia pastoris, Spodoptera frugiperda (e.g., Sf21 used in conjunction with baculovirus), Chinese hamster ovary (CHO) cells, human embryonic kidney (e.g., HEK 293) cells, HeLa cells, baby hamster kidney (BHK) cells, simian (e.g., CV-1) cells, mouse (e.g., NIH-3T3 or LTK) cells, and monkey (e.g., Cercopithecus aethiops or COS) cells. In some methods that utilize cell systems, the polypeptide-encoding nucleotide sequence is introduced such that the polypeptide-encoding nucleotide sequence copy number is stable. For example, the polypeptide-encoding nucleotide sequence can be introduced such that the polypeptide encoding nucleotide sequence is present as a stable single copy in the cell. Methods and tools for introducing polypeptide-encoding nucleotide sequences into cells are known in the art, and any such method can be used in accordance with the teachings provided herein. In an exemplary method, bacteriophage lambda can be used to insert a stable single copy of a polypeptide-encoding nucleotide sequence into E. coli. A variety of bacteriophages that can be used to insert a stable single copy of a polypeptide-encoding nucleotide sequence into a cell are known in the art, as exemplified in Simons et al., Gene (1987) 53:85-96.

Empirical measurements of translational step times and translational pause properties can be used as a substitute for statistically calculated translational kinetics values, or can supplement statistically calculated translational kinetics values. For example, empirical measurements of translational step times and translational pause properties for all 3721 codon pairs of an organism. In other examples, a sampling of codon pairs can be selected for empirical measurement in order to corroborate statistically calculated translational kinetics values. For example, codon pairs predicted to cause a translational pause, codon pairs predicted to not cause a translational pause, or a combination thereof, can be selected for empirical measurement of translational step times and translational pause properties. The results of these measurements can be used to revise the translational kinetics value of an empirically measured codon pair, and/or to evaluate the accuracy of the statistically calculated translational kinetics values. For example, a collection of codon pairs can have their translational step times and translational pause properties empirically measured, and the empirical measurements can be compared to the statistically calculated translational kinetics values, and the degree of variation between empirical measurements and calculated values can indicate the accuracy of the statistically calculated translational kinetics values. Thus, provided herein are methods of evaluating the accuracy of statistically calculated translational kinetics values, where the method comprises empirically measuring translational step times for a subset of all codon pairs, providing statistically calculated translational kinetics values for these same codon pairs, and determining the degree of correlation between empirical measurements and statistically calculated translational kinetics values, where an increased correlation is indicative of an increased accuracy of statistically calculated translational kinetics values and a decreased correlation is indicative of a decreased accuracy of statistically calculated translational kinetics values. In some embodiments, a linear correlation coefficient of at least 0.7, 0.75, 0.8, 0.85, 0.9, 0.95 or more, is indicative of statistically calculated translational kinetics values that are sufficiently accurate to predict codon pair-based translational pauses without further refinement of the statistically calculated translational kinetics values. In other embodiments, a linear correlation coefficient of 0.8, 0.75, 0.7, 0.65, 0.6, 0.55 or 0.5 or less, is indicative of statistically calculated translational kinetics values that are not sufficiently accurate to predict codon pair-based translational pauses without further refinement of the statistically calculated translational kinetics values. In such methods, the number of codon pairs to be empirically measured can be any amount sufficient to provide a sufficient comparison; for example 10, 15, 20, 25, 30, 35, 40, 50 or more codon pairs can be selected for empirical measurement. Typically, the codon pairs to be empirically measured possess a variety of different statistically calculated translational kinetics values. In one example, a combination of codon pairs predicted to cause a translational pause and codon pairs predicted to not cause a translational pause are selected for empirical measurement; in such cases, all codon pairs predicted to not cause a translational pause can have their statistically calculated translational kinetics values set to an arbitrary baseline value such as zero. In another example, a combination of codon pairs with varying degrees of being predicted to cause a translational pause is selected for empirical measurement.

In other embodiments, one or more codon pairs can be particularly selected for empirical measurement. In some instances, a particular codon pair or a few codon pairs may have statistically calculated translational kinetics values that are suspected of being inaccurate (e.g., a highly over-represented codon pair that is often located in the middle of autonomous folding units or that is not associated with other highly overrepresented codon pairs in other evolutionarily related organisms, or vice versa). In such instances, the statistically calculated translational kinetics value of such a codon pair can be checked by empirical measurement of translational step time and translational pause properties. Thus, provided herein is a method for verifying the statistically calculated translational kinetics value of a codon pair by providing a statistically calculated translational kinetics value for a codon pair, empirically measuring the translational step time for the codon pair, and determining whether or not the statistically calculated translational kinetics value of the codon pair accurately reflects the empirically measured value. Typically, when the statistically calculated translational kinetics value indicates a predicted translational pause and the empirical measurements also reflect a translational pause, the statistically calculated translational kinetics value of the codon pair can be said to accurately reflect the empirically measured value. Similarly, when the statistically calculated translational kinetics value indicates no predicted translational pause and the empirical measurements also reflect no translational pause, the statistically calculated translational kinetics value of the codon pair can be said to accurately reflect the empirically measured value. Analogously, when the statistically calculated translational kinetics value indicates a predicted translational pause and the empirical measurements reflect no translational pause, the statistically calculated translational kinetics value of the codon pair can be said to not accurately reflect the empirically measured value, and when the statistically calculated translational kinetics value indicates no predicted translational pause and the empirical measurements reflect a translational pause, the statistically calculated translational kinetics value of the codon pair can be said to not accurately reflect the empirically measured value. In various instances, the statistically calculated translational kinetics value can be replaced by or modified by the empirical measurement. For example, when the statistically calculated translational kinetics value predicts a translational pause, but no such pause was measured empirically, the statistically calculated translational kinetics value can be replaced by the empirical measurement. Similarly, when the statistically calculated translational kinetics value predicts no translational pause, but a pause was measured empirically, the statistically calculated translational kinetics value can be replaced by the empirical measurement. In other instances, when the statistically calculated translational kinetics value predicts a weak pause or a pause with low probability, but the empirical measurement indicates a strong pause, the statistically calculated translational kinetics value predicts can be modified to increase the degree to which a pause is predicted. Similarly, when the statistically calculated translational kinetics value predicts a strong pause or a pause with high probability, but the empirical measurement indicates a weak pause, the statistically calculated translational kinetics value predicts can be modified to decrease the degree to which a pause is predicted.

Calculation Methods of Modifying Translational Kinetics Values Based on Additional Translational Kinetics Data

The translational kinetics data described herein can be combined in such a manner as to provide a refined translational kinetics value for a codon pair in a host organism. Methods of combining predictive data to arrive at a refined predictive value are known in the art and can be used herein.

Estimates for translational kinetics values are informed by a number of knowledge sources known to those skilled in the art, including but not limited to experimental measurement, conservation at protein structural boundaries and across homologous families, presence or absence in highly expressed proteins, statistical inference from genomic sequence data, and the like as provided elsewhere herein. All these disparate knowledge sources must be integrated into an overall estimate for purposes of gene design and engineering. The general problem of integrating diverse and disparate knowledge sources is ubiquitous and well-studied in many different engineering fields, e.g., distributed sensor fusion in remote sensing, bagging classifiers in machine learning, heterogeneous database integration in data warehouses, or perceptual integration in artificial intelligence. Many useful and applicable approaches are known to the art.

While many approaches are possible, those skilled in the art agree that the method of Bayes [Bayes, T., 1764. An essay toward solving a problem in the doctrine of chances. Philosophical Transactions of the Royal Society of London 53:370-418. Reprinted pp. 131-153 in “Studies in the History of Statistics and Probability,” (ed. Pearson, E. S., Kendall, M. G.), Charles Griffin, London, 1970.] has rigorous foundations in probability and many success in bioinformatics [Baldi, P., and Brunak, S., 2001. Bioinformatics: The Machine Learning Approach, MIT Press, Cambridge, Mass., USA]. Using the Bayesian approach as an example here, without intending to exclude other well-known approaches, the Bayesian approach seeks to choose an hypothesis H that is most probable given the observed data D.

Operationally, this means to choose H so as to maximize the probability of H given D, written P(H|D). By Bayes's rule, this may be rewritten as P(H|D)=P(D|H)*P(H)/P(D). This is equivalent to maximizing P(D|H)*P(H) because P(D) is constant for all H. The term P(H) is identified with the degree of belief in hypothesis H before the data was observed. The term P(D|H), read “the probability of D given H,” is identified with how well hypothesis H predicts the observed data D. Thus, the Bayesian approach seeks to find an hypothesis that is a priori likely and also explains the data well.

In this example, an hypothesis H is that a given sequence feature, e.g., a given codon pair, has utility for translational kinetics engineering, e.g., creates a translational pause site. The observed data D may have several observations, e.g., D=D1 & D2 & D3 & D4, where D1=an experimental measurement, D2=conserved at protein structural domain boundaries, D3=conserved across homologous protein families, and D4=indicated as over represented by statistical analysis that yields a high chisq3 value. In this case, the term P(D|H)=P(D1 & D2 & D3 & D4|H), which indicates to choose an hypothesis that explains each of the observed datum. Of course, different data sources have different rates and magnitudes of observational error. This falls naturally into the Bayesian approach because the probability framework extends naturally to encompass the probability of observational error, as P(D|H)=P(D|H)*P(D is correct)+P(not D|H)*P(D is not correct). For example, an experimental measurement D1 that has been confirmed by replicate testing would have a very low probability of error, and therefore it would dominate the estimate if available.

In the general case, where no experimental measurement is available, several Bayesian approaches are commonly employed. The simplest, which often works well, is named “Naive Bayes” because it assumes conditional independence among the individual observed data items. In this case, P(D|H)=P(D1 & D2 & D3 & D4|H)=P(D1|H)*P(D2|H)*P(D3|H)*P(D4|H), where each of the individual terms is further expanded as P(Di|H)=P(Di|H)*P(Di is correct)+P(not Di|H)*P(Di is not correct) as indicated above. The terms P(Di is correct) and P(Di is not correct) can be estimated a priori by the correlation of Di with previous experimental measurements. The terms P(Di|H) and P(not Di|H) are obtained by observing whether or not hypothesis H is consistent with observed data item Di. More complex and powerful Bayesian approaches are also well known to the art. The fully general approach rewrites P(D|H)=P(D1 & D2 & D3 & D4|H)=P(D4|D3 & D2 & D1 & H)*P(D3|D2 & D1 & H)*P(D2|D1 & H)*P(D1|H). Many other approaches, both Bayesian and others, are well known to the art.

By way of example, the translational kinetics values for a codon pair can be refined by consideration of, for example, chi-squared value of observed versus expected codon pair frequency and the degree to which codon pairs are conserved at predicted pause sites across different proteins in the same species, for example, at protein structure domain boundaries. An over-represented codon pair which is present with above-random frequency at boundary locations between autonomous folding units of proteins in the same species can have a translational kinetics value reflecting higher predicted translational pause properties of the codon pair. In contrast, an over-represented codon pair which is present with below random frequency at boundary locations between autonomous folding units of proteins in the same species can have a translational kinetics value reflecting lower predicted translational pause properties of the codon pair.

As another example, the translational kinetics values for a codon pair can be refined by consideration of, for example, experimentally measured translation step times in one species and the degree to which codon pairs that correspond to measured pause sites in the first species are conserved across homologous proteins in other species, for example, in a multiple sequence alignment. When an over-represented codon pair in another species is aligned with above-random frequency to a codon pair that corresponds to a measured translation pause site in the first species, it can have a translational kinetics value reflecting higher predicted translational pause properties of that codon pair in the other species. In contrast, when an over-represented codon pair in another species is aligned with below random frequency to a codon pair that corresponds to a measured translation pause site in the first species, it can have a translational kinetics value reflecting lower predicted translational pause properties of that codon pair in the other species.

In various embodiments described herein, translational kinetics values for codon pairs, including refined translational kinetics values, can be determined. The translational kinetic values can be organized according to the likelihood of causing a translational pause or slowing based on any method known in the art. In one example, the translational kinetic values for two or more codon pairs, up to all codon pairs, in an organism are determined, and the mean translational kinetics value and associated standard deviation are calculated. Based on this, the translational kinetics value for a particular codon pair can be described in terms of the multiple of standard deviations the translational kinetics value for the particular codon pair differs from the mean translational kinetics value. Accordingly, reference herein to mean translational kinetics values and standard deviations, whether or not applied to a particular expression of translational kinetics value, can be applied to any of a variety of expressions of translational kinetics values provided herein.

Graphical Analysis of Translational Kinetics

Also provided herein are methods of analyzing translational kinetics of an mRNA into polypeptide encoded by a gene in a host organism by determining translational kinetics values for codon pairs in the host organism and generating a graphical display of the translational kinetics values of actual codon pairs of an original polypeptide-encoding nucleotide sequence of a heterologous gene as a function of codon position. Such a graphical display provides a visual display of the predicted translational influence, including translational pause or slowing for numerous or all codon pairs of a polypeptide-encoding nucleotide sequence. This visual display can be used in methods of modifying polypeptide encoding nucleotide sequences in order to thereby modify the predicted translational kinetics of the mRNA into polypeptide in methods such as those provided herein. For example, the graphical displays can be used to identify one or more codon pairs to be modified in a polypeptide-encoding nucleotide sequence. The graphical displays can be used in analyzing a polypeptide-encoding nucleotide sequence prior to modifying the polypeptide-encoding nucleotide sequence, or can be used in analyzing a modified polypeptide-encoding nucleotide sequence to determine, for example, whether or not further modifications are desired. The graphical displays can be created using translational kinetics values based on any of the methods for determining translational kinetics values provided herein or otherwise known in the art. For example, chi-squared as a function of codon pair position, chi-squared 2 as a function of codon position, or chi-squared 3 as a function of codon pair position, translational kinetics values thereof, empirical measurement of translational pause of codon pairs in a host organism, estimated translational pause capability based on observed presence and/or recurrence of a codon pair at predicted pause site, and variations and combinations thereof as provided herein.

The exact format of the graphical displays can take any of a variety of forms, and the specific form is typically selected for ease of analysis and comparison between plots. For example, the abscissa typically lists the position along the nucleotide sequence or polypeptide sequence, and can be represented by nucleotide position, codon position, codon pair position, amino acid position, or amino acid pair position. In such instances, the ordinate typically lists the translational kinetics value of the codon pair, such as, but not limited to, a translational kinetics value of codon pair frequency, including, but not limited to the z score of chisq1, the z score of chisq2, the z score of chisq3, the empirically measured value, and the refined translational kinetics value. In alternative embodiments, the sequence position can be plotted along the ordinate and the translational kinetics value can be plotted along the abscissa.

In another embodiment, the graphical display is a depiction of aligned related sequences, such as, for example, evolutionarily conserved sequences in different species, where the graphical display depicts the aligned sequences and the translational kinetics value of the codon pairs of each aligned sequence. For example, the graphical display can be a depiction of aligned related sequences, such as, for example, evolutionarily conserved sequences in different species, where the depiction of the sequence reflects the translational kinetics value of the codon pairs of each aligned sequence. As contemplated herein, related polypeptide-encoding nucleotide sequences can possess translational pauses that are conserved. Graphical alignment of such related sequences in a manner that reflects the translational kinetics value of the codon pairs of each aligned sequence can aid in identification of conserved translational pauses for the related sequences. Such graphical displays can be presented in any of a variety of manners. In one example, the graphical display can be an alignment of related amino acid sequences, where the translational kinetics values of each codon pair are reflected in the color of the letter representing one of the amino acids encoded by the codon pair (either the first or second amino acid encoded by the codon pair can be used, provided that the use is consistent throughout the graphical display). In this example, the translational kinetics properties information from the polypeptide-encoding nucleotide sequence can be combined with the amino acid sequence, which is used for alignment of the protein sequences, in order to provide a graphical display of conservation of nucleotide sequence-dependent translational pauses as function of amino acid sequence. In another example, the graphical display can be an alignment of related amino acid sequences, where the translational kinetics values of each codon pair are reflected in the font size of the letter representing one of the amino acids encoded by the codon pair. In another example, the graphical display can be a three-dimensional graph displaying translational kinetics values along the vertical axis, codon pair position along one horizontal axis, and different related sequences along a second horizontal axis. Any of a variety of additional graphical methods for such analysis consistent with the teachings provided herein is readily available to one skilled in the art.

Graphical displays depicting aligned sequences and the translational kinetics value of the codon pairs of each aligned sequence can be used to compare the codon pair translation kinetics values of a one or more proteins, such as, for example, a selected gene to be expressed, with gene sequences related to each other, such as gene sequences related at least a part of the selected gene sequence. Related gene sequences that can be used in such a comparison include related gene family members in the same species or in different species. Related genes of interest also include specific homologous portions of other genes such as conserved domain elements. In addition, related genes of interest can include portions of genes that are characterized by three dimensional structures that share a common protein domain structure with each other. A significant number of conserved domain elements have been characterized and the locations of homologous domains documented in alternative proteins within and across species. Gene family relationships, the locations of protein domain elements, and proteins that share related folds can be identified by one skilled in the art, based on information available in public databases such as, for example, UniProt, Pfam, Propom, Interpro, SCOP, PFD, ExPasy, EBI, Ensembl, the various databases at NCBI such as Genbank and COG as well as other, more specialized publicly available protein and nucleic acid sequence databases. Typically, related genes to be aligned with each other refers to genes that are classified as belonging to the same structural class, as identified by any publicly available resource for structural classification, such as, for example, SCOP, and/or genes having at least 50%, 60%, 70%, 80%, 90%, 91%, 92%, 93%, 94%, 95%, 96%, 97%, 98% or 99% sequence identity with each other or with one particular gene in the group. In some embodiments, related sequences are selected by identifying a group of known amino acid sequences that share some sequence identity with a query amino acid sequence (using, e.g., a tool such as BLAST which can identify homologous amino acid sequence), and of this group, selecting amino acid sequences from a variety of diverse organisms by selecting an amino acid sequence from each of several organisms where the selected amino acid sequence has the highest degree of homology to the query amino acid sequence of any protein for that organism, where the number of organisms included can be 2, 3, 4, 5, 6, 7, 8, 9, 10 or more different organisms, typically from at least different genera, different families, different orders, or from different classes. In other embodiments, related sequences are selected by identifying a group of known amino acid sequences that share some sequence identity with a query amino acid sequence (using, e.g., a tool such as BLAST which can identify homologous amino acid sequence), and of this group, selecting amino acid sequences from a variety of diverse organisms where the selected amino acid sequences can be confirmed as sharing the same protein fold as can be verified for protein folds with known conserved amino acid sequence properties, and as is known in the art.

The choice of sequences for comparison with each other in graphical displays is guided by the number and degree of similarity of alternative sequences sufficient to provide a desired representative sample of related proteins and domains. A representative sample refers to a sample with a sufficient amount of evolutionary divergence so that the key conserved pause sites that play a role in proper protein folding, biological processing, and localization of the proteins are conserved among most or all members of the set used for comparison while other pause sites are not conserved. For analyzing sequences within a species, at least two alternative family members are typically selected for comparison. When larger numbers of related family members exist, more representatives can be included, such as 3, 4, 5, 6, 7, 8, 9, 10 or more family members, in the comparison. Typically, family members are selected based on their relative degrees of homology, so that a wide sequence variety of related family members are selected. The degree of relationship between two genes can be measured, for example by known computational algorithms that calculate the amount of homology between two sequences. In some embodiments, sequences are selected that include two or more species, and typically span a broad range across the phylogenetic tree. For example, a useful range of species for comparison with a human gene, could include related genes from mouse, Drosophila, nematode, Arabidopsis, yeast, E. coli, and combinations thereof. Selection of one or more species to be included can be made according to factors such as the availability of related sequences, desired variation between compared sequences, and number of sequences to be included in the comparison, as will be recognized by one skilled in the art. Related sequences can be aligned with each other using known methods and tools such as ClustalW.

Once sequences for comparison have been selected, the codon pair translation kinetics value of each codon pair for each sequence is graphically displayed, whereupon the graphical display can be analyzed to identify and locate potential translation pause sites. Potential pause sited can be indicated on the graphical display by any of a variety of methods such as those provided above. In one example, amino acid sequences of each sequence are depicted in an alignment (e.g., similar to a typical ClustalW output), and a translational values can be reflected by modifying the color or font of the amino acid according to the translational kinetics value of the corresponding codon pair. Also, contemplated herein, analysis of these sequences to identify potential translation pause sites can be performed, for example, using the statistical methods for determining codon pair biases such as expectation maximization methods, as provided elsewhere herein or otherwise known in the art. Such statistical comparison of aligned sequences can be performed using a computer, for example a computer running programmatic scripts that search through aligned sequence data for conserved pause sites and output the locations of such sites. The result of the sequence analysis, graphical or statistical, of each of the genes selected for comparison is a list of likely translation pause sites, as described below.

Likely translation pause sites can be identified based on determination of predicted pause sites conserved in the aligned gene sequences. Conserved pause sites can be recognized as pause sites that occur in the same or similar aligned location within the genes in most or all related sequences. In some cases, conserved pause sites will not be at precisely the same aligned amino acid position, but rather can be recognized as being in approximately the same position. For example, conserved pause sites can be identified when predicted pause sites for most or all sequences are present within or within about 5, 4, 3, 2, or 1 aligned amino acids. This permits identification of a conserved pause despite variability between genes due to deletions or insertions resultant from evolutionary divergence of the sequences. Typically, although the exact position of the predicted pause sites can vary slightly, the amino acid context flanking the conserved pause sites will remain the same. When the amount of amino acid homology between related proteins is very low, the conserved pause sites can be recognized as occurring in structurally similar portions of the protein. In some embodiments where the degree of sequence similarity between related genes being compared is very low, the graphical display also contains a depiction of structural features of the proteins, such as information from X-ray crystallography or from computational algorithms that predict protein domains and/or secondary structures.

Conserved pause sites can occur before the start of an autonomous folding unit, or after the end of an autonomous folding unit. In some embodiments, conserved pause sites may occur within an autonomous folding unit of a protein. Such pause sites may occur, for example, in structural turn regions of an autonomous folding unit. Thus, superimposing known or predicted protein structural elements, for example secondary structure or domain features, on the graphical displays provided herein can assist in identifying such functionally important pause sites.

The result of the graphical or statistical comparison of the related genes is a list of conserved pause sites, within a canonical gene or a gene selected for expression, which are conserved across a range of phylogenetic groups and/or across divergent related proteins. These conserved pause sites can be selected as candidates for inclusion in a modified polypeptide-encoding nucleotide sequence in accordance with the methods provided elsewhere herein. These conserved pause sites also can be used to modify the translational kinetics value for codon pairs located at the site of the conserved translational pause, where a the translational kinetics value of a codon pair located at the site of the conserved translational pause can be modified to increase the likelihood that this codon pair causes a translational pause, in accordance with the methods provided elsewhere herein.

The graphical displays provided herein can represent the predicted translational kinetics of a polypeptide-encoding nucleotide sequence in a particular organism. The polypeptide-encoding nucleotide sequence can be any nucleotide sequence, such as, for example, a wild type sequence, a mutant sequence found in nature, a mutant or otherwise modified sequence caused by human activities (e.g., breeding or mutagenic methods), or a synthetic sequence in which the nucleotide sequence is derived and/or optimized (e.g., in a computer) according to the amino acid sequence (and optionally additional parameters), and may or may not have homology to other polypeptide-encoding nucleic acids. The organism can be the native host organism or a heterologous organism, relative to the polypeptide to be expressed.

Thus, some embodiments provided herein include graphical displays and related methods, where the predicted translational kinetics are graphically displayed for a wild type polypeptide-encoding nucleotide sequence expressed in the native host organism. Also provided herein are graphical displays of predicted translational kinetics for a wild type polypeptide-encoding nucleotide sequence expressed in a heterologous host organism. Also provided herein are graphical displays of predicted translational kinetics for a modified or synthetic polypeptide-encoding nucleotide sequence expressed in the wild type host organism. Also provided herein are graphical displays of predicted translational kinetics for a modified or synthetic polypeptide-encoding nucleotide sequence expressed in a heterologous host organism.

Comparing Plots

Also contemplated herein are methods in which a set of graphical displays, including at least a first graphical display and a second graphical display, are prepared. These sets of displays can be compared in order to determine the difference in predicted translational efficiency or translational kinetics of the two plots. The plots can differ according to any of a variety of criteria. For example, each plot can represent a different polypeptide-encoding nucleotide sequence, each plot can represent a different host organism, each plot can represent differently determined translational kinetics values, or any combination thereof. As will be apparent to one skilled in the art, any number of different graphical displays can be compared in accordance with the methods provided herein, for example, 2, 3, 4, 5, 6, 7, 8 or more different graphical displays can be compared. Typically, two plots will represent different polypeptide-encoding nucleotide sequences, the same sequence in different host organisms, or different sequences in different host organisms.

Comparison of different graphical displays can be used to analyze the predicted change in translational kinetics as a result of the difference represented by the graphical displays. For example, comparison of the same polypeptide-encoding nucleotide sequence in different host organisms can be used to analyze any predicted changes in translational kinetics between the two organisms. In one example, the polypeptide-encoding nucleotide sequence in the native host organism can be compared to the same polypeptide encoding nucleotide sequence in a heterologous host organism, and any predicted changes in translational kinetics between the two organisms can be analyzed. Comparisons also can be made of different polypeptide-encoding nucleotide sequences in a particular host organism in order to analyze any predicted changes in translational kinetics as a result of differences in the polypeptide-encoding nucleotide sequence. In one example, the wild-type polypeptide encoding nucleotide sequence in a heterologous host organism can be compared to a modified polypeptide-encoding nucleotide sequence in the same heterologous host organism, and any predicted changes in translational kinetics between the two sequences can be analyzed. In such instances, the encoded polypeptide sequences can be the same or can be different. Comparisons also can be made of different polypeptide-encoding nucleotide sequences in different host organisms in order to analyze any predicted changes in translational kinetics as a result of these differences. In one example, the wild-type polypeptide-encoding nucleotide sequence in the native host organism can be compared to a modified polypeptide-encoding nucleotide sequence in a heterologous host organism, and any predicted changes in translational kinetics between the two can be analyzed. With computer-generated synthetic polypeptide-encoding nucleotide sequences, random (non optimized) codon pair selection can be compared with more optimized selection based on native codon pair preferences of the expression organism.

In preferred embodiments, graphical displays of translational kinetics values of codon pairs in a host organism are plotted as a function of polypeptide-encoding nucleotide sequence. The graphical displays provided herein reflect the predicted or estimated influence on translational kinetics by each codon pair in an organism, thereby facilitating analysis of translational kinetics of an mRNA into polypeptide by comparing graphical displays of different codon pairs in sequences encoding the polypeptide. Previous graphical methods did not include improved translational kinetics values, and, therefore the resultant graphical displays provided information that might have been inadequate in depicting the actual translational kinetics of the polypeptide-encoding nucleotide. In addition, previous graphical methods did not compare translational kinetics values of codon pair frequencies, and, therefore the resultant graphical displays did not provide a basis to determine the relative degree to which codon pairs were biased (e.g., over-represented or under-represented). Without providing such graphical representations, it was not possible to establish standard methods of deriving suitable cutoffs for determining when a codon pair is sufficiently biased so as to be identified as likely influencing translational kinetics. These shortcomings also have led to the inability to provide graphical displays that can be readily analyzed for purposes of determining whether or not a polypeptide-encoding nucleotide sequence is predicted to have suitable translational kinetics, and if not, selecting particular codon pairs to be modified in attempting to derive a polypeptide-encoding nucleotide sequence with suitable predicted translational kinetics. It has been determined herein that graphical displays containing improved translational kinetics values of codon pairs permit determinations of suitable cutoffs and permits accurate selection of candidate codon pairs for modification, to an extent that has heretofore not been readily accessible.

Accordingly, provided herein are methods of analyzing translational kinetics of an mRNA into polypeptide in a host organism by comparing two graphical displays to understand or predict the differences in translational kinetics of the mRNA into polypeptide, where the differences in the graphical displays can be as a result of, for example, a difference in the polypeptide-encoding nucleotide sequence or a difference in the host organism. Upon determination of the differences in translational kinetics, it can be evaluated whether or not the change in translational kinetics as a result of the underlying difference between the two graphical displays is desirable. Such comparison methods also can lead to an identification of further modifications, e.g., further modifications to the polypeptide-encoding nucleotide sequence to further improve translational kinetics. Accordingly, it is contemplated herein that such comparison methods can be carried out iteratively.

In embodiments where it is desired to express a polypeptide-encoding nucleotide sequence in a particular heterologous host, a graphical display of the translational kinetics values of codon pairs in the native host can be compared to a graphical display of the translational kinetics values of codon pairs in the heterologous host, and codon pairs can be identified that can be modified in order to change the translational kinetics of the mRNA into polypeptide in a desired fashion. One or more proposed modifications to the polypeptide encoding nucleotide sequence can be generated, and graphical displays can be prepared for the translational kinetics values of codon pairs in the modified polypeptide-encoding nucleotide sequences in the heterologous host organism. A graphical display of a modified polypeptide-encoding nucleotide sequence can be compared to the graphical display of the unmodified, original polypeptide-encoding nucleotide sequence expressed in the host organism and/or to the graphical display of the unmodified, original polypeptide-encoding nucleotide sequence expressed in the heterologous organism. Comparison of these graphical displays provides a convenient visual basis for determining whether or not the change in translational kinetics is desirable, and as a result determining whether or not the modification to the polypeptide-encoding nucleotide sequence is desirable.

In embodiments where it is desired to improve expression of a polypeptide-encoding nucleotide sequence in a particular heterologous host, a graphical display of the translational kinetics values of codon pairs for the original polypeptide encoding nucleotide sequence in the heterologous host can be compared to a graphical display of the translational kinetics values of codon pairs for a modified polypeptide encoding nucleotide sequence in the heterologous host, and it can be determined whether or not the modification to the polypeptide-encoding nucleotide sequence resulted in improved translational kinetics.

In embodiments where it is desired to select a suitable heterologous host for expression of a polypeptide-encoding nucleotide sequence, a graphical display of the translational kinetics values of codon pairs for the polypeptide-encoding nucleotide sequence in the native host can be compared to a graphical display of the translational kinetics values of codon pairs for the polypeptide-encoding nucleotide sequence in one or more heterologous hosts, and the graphical displays can be compared to identify any host organism(s) with preferred translational kinetics.

In embodiments where modifying and/or replacing a codon pair predicted to cause a translational pause with a codon pair not predicted to cause a translational pause is performed, desirability of such a modification and/or replacement can be evaluated based on the location along the nucleotide sequence of one or more codon pairs predicted to cause a translational pause or slowing, using a graphical display of the translational kinetics values of codon pairs for the polypeptide-encoding nucleotide sequence. As provided herein, codon pairs causing translational pauses, such as over-represented codon pairs, closer to the amino terminus/translation initiation site can have a stronger influence on protein expression levels compared to codon pairs causing translational pauses that are situated further downstream (i.e., closer to the carboxy terminus). Accordingly, a graphical display can be used to determine the location of one or more codon pairs predicted to cause a translational pause or slowing, and the proximity of such codon pairs to the amino terminus/translation initiation site can be considered in determining what, if any, modification to make to the polypeptide encoding nucleotide sequence.

In another embodiment, as provided herein above, graphical displays of aligned related genes can be used to compare the aligned sequences and identify conserved pause sites. One or more of these conserved pause sites can be selected as candidates for inclusion in a modified polypeptide-encoding nucleotide sequence in accordance with the methods provided elsewhere herein.

Changes to Translational Kinetics

The methods and graphical displays provided herein permit one to modify translational kinetics of an mRNA into polypeptide. Translational kinetics of an mRNA into polypeptide can be changed in order to achieve any of a variety of expression profiles. For example, translational kinetics of an mRNA into polypeptide can be changed in order to more closely resemble the translational kinetics of the mRNA into polypeptide in the native host organism. In another example, translational kinetics of an mRNA into polypeptide can be changed in order to replace some or all codon pairs that cause a translational pause with codon pairs that do not cause a translational pause. In another example, translational kinetics of an mRNA into polypeptide can be changed in order to replace some or all codon pairs that cause a translational pause and that are predicted to occur within an autonomous folding unit of a nascent protein with codon pairs that do not cause a translational pause. In another example, translational kinetics of an mRNA into polypeptide can be changed in order to include or preserve, at least approximately, one or more translational pauses, such as, for example, translational pauses predicted to occur before, after, or between autonomous folding units of a nascent protein. In another example, determination of inclusion or exclusion of translational pauses before, after, or between autonomous folding units of a nascent protein can be based on a comparison of the predicted translational kinetics (e.g., using one or more graphical displays) of two or more related proteins from the same or different species. In another example, translational kinetics of an mRNA into polypeptide can be changed in order to replace some or all under-represented codon pairs with codon pairs that are not under-represented. In another example, translational kinetics of an mRNA into polypeptide can be changed in order to replace all codon pairs that cause a translational pause with codon pairs that do not cause translational pauses.

In some embodiments, translational kinetics of an mRNA into polypeptide is changed in order to more closely resemble the translational kinetics of the mRNA into polypeptide in the native host organism. As used herein, a change of translational kinetics to more closely “resemble” the translational kinetics of the native host organism refers to a change in translational kinetics of an mRNA into polypeptide in a heterologous host organism that modifies a codon pair such that a translational pause is present at or near the site of a translational pause for expression of the nascent polypeptide in the native host organism, and/or modifies a codon pair such that no translational pause is present when a translational pause is not present in the expression profile of the polypeptide in the native host organism. Typically, more than one codon pair is changed in the polypeptide-encoding nucleotide sequence, such that one or more translational pauses are no longer present, one or more translational pauses are introduced, or one or more translational pauses are no longer present and one or more translational pauses are introduced. It is contemplated herein that a change in translational kinetics of an mRNA into polypeptide in order to resemble the translational kinetics of the mRNA into polypeptide in the native host organism will, for at least some polypeptides, increase levels of expression of the polypeptide, increase levels of expression of properly folded polypeptide, increase levels of expression of soluble polypeptide, and/or increase levels of properly post-translationally processed polypeptide.

In some instances, it is not possible to modify the polypeptide-encoding nucleotide sequence such that a translational pause is not present in the expression profile of the polypeptide in the native host organism. For example, there may be no codon pairs that are not predicted to cause a translational pause or slowing and that encode a corresponding pair of amino acids. In such instances, several options are available: the codon pair that is least likely to cause a translational pause or slowing can be selected; an amino acid insertion, deletion or mutation can be introduced to yield a codon pair that is not predicted to cause a translational pause or slowing; or no change is made. One option in a computerized method is to request human input in order to resolve the issue. Alternatively, the computer may be programmed to make a selection. In methods in which an amino acid insertion, deletion or mutation is made in order to change translational kinetics, it is preferable to select a change that is predicted not to substantially influence the final three-dimensional structure of the protein and/or the activity of the protein. Such an amino acid mutation can include, for example, a conservative amino acid substitution such as the conservative substitutions shown in Table 1. The substitutions shown are based on amino acid physical-chemical properties, and as such, are independent of organism. In some embodiments, the conservative amino acid substitution is a substitution listed under the heading of exemplary substitutions.

TABLE 1

| Original | Conservative | Exemplary |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe | leu |
| Leu (L) | ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala | leu |

While in some embodiments, all codon pairs predicted to cause a translational pause or slowing are treated equally, in other embodiments, one or more different threshold levels can be established for differential treatment of codon pairs, where codon pairs above a highest threshold are the codon pairs most likely to cause a translational pause or slowing, and succeedingly lower codon pair threshold-based groups correspond to succeedingly lower likelihoods of the respective codon pairs causing a translational pause or slowing. Based on the codon pair groupings, different numbers or percentages of codon pairs can be removed for each of these different threshold-based groups. For example, 95% or more codon pairs above a highest threshold level can be removed, while 90% or less of all codon pairs between that level and an intermediate threshold level are removed. As contemplated herein, codon pairs likely to cause a translational pause or slowing can be segregated into two or more different threshold-based groups, three or more different threshold-based groups, four or more different threshold-based groups, five or more different threshold-based groups, six or more different threshold-based groups, or more. Discussion of specific thresholds are provided elsewhere herein; however, typically the higher the threshold, the higher the likelihood of a translational pause or slowing caused by a codon pair with a translational kinetics value greater than the threshold. In embodiments in which codon pairs likely to cause a translational pause or slowing can be segregated into two or more different threshold-based groups, different numbers or percentages of codon pairs can be removed for each codon pair group. For example, in one embodiment, at least 10%, 20%, 30%, 40%, 50%, 60%, 70%, 75%, 80%, 85%, 90%, 95%, 97%, 98% or 99% of codon pairs above a highest threshold are removed, while the same or a lower percentage of codon pairs are removed from codon pair groups corresponding to one or more lower thresholds. Typically, for each successively lower threshold group, the same or a lower percentage of codon pairs are removed. In one example, all codon pairs above a highest threshold are removed, while a codon pair above an intermediate threshold is removed only if the codon pair is located within an autonomous folding unit. In another example, all codon pairs above a highest threshold are removed, while a codon pair above an intermediate threshold is removed only if the codon pair can be removed without requiring a change in the encoded polypeptide sequence. In another example, all codon pairs above a highest threshold are removed, while a codon pair above a first higher intermediate threshold is removed only if the codon pair can be removed without changing the encoded polypeptide sequence or with only a conservative change to the encoded polypeptide sequence, while a codon pair above a second lower intermediate threshold is removed only if the codon pair can be removed without requiring any change in the encoded polypeptide sequence. While the above discussion has been applied to the use of a plurality of threshold levels, it will be readily apparent to one skilled in the art that, in the place of using threshold levels, an evaluation method can be used that determines the degree to which a codon pair should be removed according to the translational kinetics value of the codon pair, where the degree to which the codon pair should be removed can be counterbalanced by any of a variety of user-determined factors such as, for example, presence of the codon pair within or between autonomous folding units, and degree of change to the encoded polypeptide sequence.

In some instances, it is not possible to modify the polypeptide-encoding nucleotide sequence to introduce a translational pause at the site of a translational pause for expression of the polypeptide in the native host organism. For example, there may be no codon pairs predicted to cause a translational pause or slowing and encoding a corresponding pair of amino acids. In such instances, several options are available: the codon pair that is most likely to cause a translational pause or slowing can be selected; the polypeptide encoding nucleotide sequence can be scanned upstream and downstream of the codon pair site in question, and a nearby codon pair can be changed to a codon pair predicted to cause a translational pause or slowing; an amino acid insertion, deletion or mutation can be introduced to yield a codon pair that is predicted to cause a translational pause or slowing; or no change is made. In methods in which the polypeptide-encoding nucleotide sequence can be scanned upstream and downstream of the codon pair site in question, modifications to codon pairs closer to the codon pair site in question is typically preferred to more distant modifications, and modifications are typically avoided that introduce a translational pause where it is not desired (e.g., within an autonomous folding unit of a protein) or that modify a codon pair such that a translational pause is not present where a translational pause is desired (e.g., between autonomous folding units of a protein). For example, one of the 1, 2, 3, 4 or 5 most proximal codon pairs upstream (5′ of the desired pause site) or one of the 1, 2, 3, 4 or 5 most proximal codon pairs downstream (3′ of the desired pause site) can be chosen for replacement to introduce the translational pause or slowing. Typically in such instances, 1 codon pair upstream or downstream is selected favor of 2 codon pairs upstream or downstream, provided the desired translational pause or slowing can be attained. In methods in which an amino acid insertion, deletion or mutation is made in order to change translational kinetics, it is preferable to select a change that is predicted not to substantially influence the final three-dimensional structure of the protein and/or the activity of the protein. Such an amino acid mutation can include, for example, a conservative amino acid substitution such as the conservative substitutions shown in Table 1.

In some embodiments, translational kinetics of an mRNA into polypeptide can be changed in order to replace some or all codon pairs that cause translational pauses or other codon pairs that cause translational slowing with codon pairs that do not cause translational pauses or translational slowing. While not intending to be limited to the following, it is believed that, for at least some proteins, reduction or elimination of the number of translational pauses that occur during translation can serve to increase the expression level and/or quality of the protein. Accordingly, by replacing some or all codon pairs that cause translational pauses or other codon pairs that cause translational slowing with codon pairs that do not cause translational pauses or translational slowing, the expression levels and/or quality of an expressed protein can be increased. Thus, also provided herein are polypeptide-encoding nucleotide sequences that have been modified to have one or more codon pairs that cause a transcription pause or slowing replaced with codon pairs that are less likely to cause a translational pause or slowing. While in some embodiments it is preferred to replace all codon pairs predicted to cause a translational pause or slowing, in other embodiments, it is sufficient to replace a subset of codon pairs predicted to cause a translational pause or slowing. For example, expression levels can be increased by replacing at least 1, 2, 3, 4, 5, 6, 7, 8, 9, 10 or more codon pairs predicted to cause a translational pause or slowing. In another example, at least 10%, 20%, 30%, 40%, 50%, 60%, 70%, 75%, 80%, 85%, 90%, 95%, 97%, 98% or 99% of codon pairs predicted to cause a translational pause or slowing are replaced by, for example, substituting different codon pairs that encode the same amino acids.

In accordance with the methods and graphical displays provided herein, a translational kinetics value of a codon pair is a representation of the degree to which it is expected that a codon pair is associated with a translational pause. Methods of determining the translational kinetics value of a codon pair are discussed elsewhere herein. Such translational kinetics values can be normalized to facilitate comparison of translational kinetics values between species. In some embodiments, the translational value can be the degree of over-representation of a codon pair. An over-represented codon pair is a codon pair which is present in a protein-encoding sequence in higher abundance than would be expected if all codon pairs were statistically randomly abundant. When translational kinetics translational kinetics values of codon pairs are determined, a codon pair predicted to cause a translational pause or slowing is a codon pair whose likelihood of causing a translational pause or slowing is at least one standard deviation above the mean likelihood of causing a translational pause or slowing. In the methods provided herein, a threshold for the translational kinetics value of codon pairs that are predicted to cause a translational pause or slowing can be set in accordance with the method and level of stringency desired by one skilled in the art. For example, when it is desired to identify only a small number of the codon pairs most likely to cause a translational pause or slowing, a threshold value can be set to 5 standard deviations or more above the mean translational kinetics value. Typical threshold values can be at least 1, 1.25, 1.5, 1.75, 2, 2.25, 2.5, 3, 3.5, 4, 4.5 and 5 standard deviations above the mean. In one example, the threshold value is 3 standard deviations above the mean. As provided herein, a plurality of thresholds can be applied in the herein provided methods in segregating codon pairs into a plurality of groups. Each threshold of such a plurality can be a different value selected from 1, 1.25, 1.5, 1.75, 2, 2.25, 2.5, 3, 3.5, 4, 4.5 and 5 standard deviations above the mean.

In some embodiments, translational kinetics of an mRNA into polypeptide can be changed in order to include or preserve one or more translational pauses. A translational pause can serve to slow translation of the nascent amino acid chain. In some instances when such translational pauses are desirable, for example, in instances in which a pause(s) serves to facilitate proper polypeptide folding, post-translational modification, re-organization/folding at protein domain boundaries, one or more translational pauses can be included in the modified polypeptide-encoding nucleotide sequence. Such pauses can be, for example, pauses that are preserved, referring to a pause that is present in the original polypeptide-encoding nucleotide sequence when expressed in the native organism being also present in the modified polypeptide-encoding nucleotide sequence for the intended host organism. Such pauses can be, for example, pauses that are conserved among related polypeptide-encoding nucleotide sequences, referring to a pause that is present in most or all of a number of sequences related to the polypeptide-encoding nucleotide sequence to be expressed, where methods of comparing related sequences and identifying conserved pauses are provided in more detail elsewhere herein. Such pauses also can be inserted, for example, when the intended host organism encodes a homologous protein and the polypeptide encoding nucleotide sequence of the homologous protein contains one or more translational pauses, the modified polypeptide-encoding nucleotide sequence also can contain one or more of such translational pauses of the homologous protein from the host organism. In some such embodiments, the polypeptide-encoding nucleotide sequence can be modified to contain the codon pair associated with the translational pause from the homologous protein in the host organism. In some embodiments, the polypeptide-encoding nucleotide sequence can be modified to contain a codon pair that causes a translational pause in order to intentionally down regulate or reduce the expression level of the encoded polypeptide. Additionally, pause(s) can be inserted at any particular location in the modified polypeptide-encoding nucleotide sequence for any of a variety of reasons one skilled in the art may have for slowing translational speed at a particular site.

In some embodiments provided herein, one or more pauses that are predicted to be present in native translation of the original polypeptide-encoding nucleotide sequence is/are preserved in a modified polypeptide-encoding nucleotide sequence provided in accordance with the teachings herein. For example, a codon pair in the modified polypeptide-encoding nucleotide sequence can be selected to have a predicted translational kinetics value that is at least 50%, 60%, 70%, 75%, 80%, 85%, 90%, 95%, 97%, or 99% that of the native codon pair whose predicted pause is to be preserved; further, the codon pair in the modified polypeptide-encoding nucleotide sequence can be selected to be located within 30, 20, 10, 9, 8, 7, 6, 5, 4, 3, 2 or 1 codons of the native codon pair whose predicted pause is to be preserved.

In some embodiments, the translational kinetics of an mRNA into polypeptide can be changed in order to include or preserve one or more translational pauses predicted to occur before, after, within, or between autonomous folding units of a protein.

While not intending to be limited to the following, it is proposed that translational pauses are present in wild type genes in order to slow translation of a nascent polypeptide subsequent to translation of a protein domain, thus providing time for acquisition of secondary and at least partial tertiary structure in the domain prior to further downstream translation. By modifying the translational kinetics of complex multi-domain proteins it may be possible to experimentally alter the time each domain has available to organize. Folding of a heterologously expressed gene having two or more independent domains can be altered by the presence of pause sites between the domains. Refolding studies indicate that the time it takes for a protein to settle into its final configuration may take longer than the translation of the protein. Pausing may allow each domain to partially organize and commit to a particular, independent fold. Other co-translational events, such as those associated with co factors, protein subunits, protein complexes, membranes, chaperones, secretion, or proteolytic complexes, also can depend on the kinetics of the emerging nascent polypeptide. Pauses can be introduced by engineering one codon pair predicted to cause a translational pause or slowing, or two or more such codon pairs into the sequence to facilitate these co translational interactions.

As such, provided herein is the recognition that the presence of codon pairs predicted to cause a translational pause or slowing in protein-encoding regions separating regions encoding different autonomous folding units of the protein can serve to pause translation and facilitate folding of the nascent translated protein, where autonomous folding units can be secondary structural elements such as an alpha helix, or can be tertiary structural elements such as a protein domain. Accordingly, provided herein are methods of changing translational kinetics of an mRNA into polypeptide by including or preserving one or more translational pauses predicted to occur before, after, or between autonomous folding units of a protein, thereby increasing the likelihood that the translated protein will be properly folded. In such embodiments, typically a translational pause is preserved, which refers to maintaining the same codon pair for a polypeptide-encoding nucleotide sequence that is expressed in the native host organism, or, when the polypeptide-encoding nucleotide sequence is heterologously expressed, changing the codon pair as appropriate to have a translational kinetics value comparable to or closest to the translational kinetics value of the native codon pair in the native host organism.

In another example, determination of inclusion or exclusion of translational pauses before, after, or between autonomous folding units of a nascent protein can be based on a comparison of the predicted translational kinetics (e.g., using one or more graphical displays) of two or more related proteins from the same or different species. As provided herein, the number and/or position of translational pauses predicted to occur before, after, or between autonomous folding units of a protein can be determined using the methods provided herein for comparing predicted translational kinetics for two or more related proteins. For example, graphical displays of native expression for two or more related proteins can be compared and the number and/or position of predicted translational pauses conserved across the proteins can be determined. Based on this determination of conserved predicted translational pauses, methods that include changing the translational kinetics of an mRNA into polypeptide can include preserving one or more, or all conserved predicted translational pauses, particularly those present between autonomous folding units of a nascent protein.

In some embodiments, translational kinetics of an mRNA into polypeptide can be changed in order to replace some or all codon pairs predicted to cause translational pauses and that are predicted to occur within an autonomous folding unit of a protein, with codon pairs not predicted to cause a translational pause. As used herein, an autonomous folding unit of a protein refers to an element of the overall protein structure that is self stabilizing and often folds independently of the rest of the protein chain. Such autonomous folding units typically correspond to a protein domain. As provided herein, expression of a gene in a heterologous host organism can result in translational pauses located in regions that inhibit protein expression and/or protein folding. Since the presence of codon pairs predicted to cause a translational pause or slowing in protein-encoding regions separating regions encoding different autonomous folding units of the protein can serve to pause or slow translation and, in some instances, facilitate folding of the nascent translated protein, and thereby increase the likelihood that the translated protein will be properly folded, it is also contemplated that replacing codon pairs predicted to cause translational pauses within an autonomous folding unit of a protein, particularly for heterologously expressed proteins, with codon pairs not predicted to cause a translational pause can result in improved expression levels and/or folding of expressed proteins. Accordingly, provided herein are methods of changing translational kinetics of an mRNA into polypeptide by replacing some or all codon pairs predicted to cause translational pauses and that are predicted to occur within an autonomous folding unit of a protein with codon pairs not predicted to cause a translational pause, thereby increasing expression levels and/or improving the folding of expressed proteins.

In the methods provided herein that include changing translational kinetics of an mRNA into polypeptide by modifying codon pairs with regard to their location within or outside of autonomous folding units of proteins, one step can include identifying predicted autonomous folding units of a protein. Methods for identifying predicted autonomous folding units of a protein or protein domains are known in the art, and include alignment of amino acid sequences with protein sequences having known structures, and threading amino acid sequences against template protein domain databases. Such methods can employ any of a variety of software algorithms in searching any of a variety of databases known in the art for predicting the location of protein domains. The results of such methods will typically include an identification of the amino acids predicted to be present in a particular domain, and also can include an identification of the domain itself, and an identification of the secondary structural element, if any, in which each amino acid sequence of a domain is located.

Some methods provided herein include evaluating whether or not to modify and/or replace a predicted translational pause. In such methods desirability of such a modification and/or replacement can be evaluated based on the location along the nucleotide sequence of one or more codon pairs predicted to cause a translational pause or slowing. Such evaluation can be performed for example, using a graphical display of the translational kinetics values of codon pairs for the polypeptide-encoding nucleotide sequence, or by other computational methods provided herein or otherwise known in the art. As provided herein, over-represented codon pairs closer to the amino terminus/translation initiation site can have a stronger influence on protein expression levels compared to over-represented codon pairs situated further downstream (i.e., closer to the carboxy terminus). Accordingly, the location of one or more codon pairs predicted to cause a translational pause or slowing relative to the amino terminus/translation initiation site can be considered in determining what, if any, modification to make to the polypeptide-encoding nucleotide sequence, where an increasing proximity to the amino terminus/translation initiation site will typically correspond to an increasing predicted translational pause or slowing effect of the codon pair. Thus, in instances in which replacement of a codon pair predicted to cause translational pause or slowing with a codon pair not predicted to cause a translational pause or slowing is desired, an increasing proximity to the amino terminus/translation initiation site will typically correspond to an increasing desirability to modify and/or replace the codon pair. Such evaluation can find particular application in embodiments in which a predicted translational pause or slowing can be replaced only by modification (e.g., addition, deletion or mutation) of the encoded amino acid sequence, where the proximity to the amino terminus/translation initiation site of a codon pair predicted translational pause or slowing can serve as a weighting factor (e.g., increasing in importance with increasing proximity to the amino terminus/translation initiation site and decreasing in importance with increasing distance away from the amino terminus/translation initiation site) in evaluating whether or not to modify the amino acid sequence, particularly in instances in which it is desirable to not modifying the encoded amino acid sequence or only conservatively modify the amino acid sequence (e.g., by a conservative amino acid substitution). Similar sequence location-based weighting of the importance of modification and/or replacement of a codon pair predicted to cause translational pause or slowing with a codon pair not predicted to cause a translational pause or slowing can be applied to any of a variety of other factors considered when modifying or otherwise designing a polypeptide-encoding nucleic acid sequence. For example, when a synthetic polypeptide-encoding nucleic acid sequence is generated, a variety of factors can be considered (as provided elsewhere herein), where one such factor is the predicted translational pause or slowing properties of a codon pair. In such instances, the predicted translational pause or slowing properties of a codon pair can be further weighted by the location of the codon pair along the polypeptide-encoding nucleotide sequence such that the predicted influence on translational pause or slowing increases with increasing proximity to the amino terminus/translation initiation site and the predicted influence on translation pause or slowing decreases with increasing distance away from the amino terminus/translation initiation site.

In some embodiments, there may be two or more candidate pauses to introduce into the polypeptide-encoding nucleotide sequences. In such embodiments, the two or more different polypeptide-encoding nucleotide sequences can be generated where the different polypeptide-encoding nucleotide sequences differ by the number of and/or placement of translational pauses. One of these different polypeptide-encoding nucleotide sequences can contain all candidate pauses; one of these different polypeptide-encoding nucleotide sequences can contain none of the candidate pauses. In some embodiments, all possible combinations of candidate pauses are prepared. The various different polypeptide encoding nucleotide sequences can be tested according to known expression and protein assay methods to determine which polypeptide-encoding nucleotide sequence(s) is most suitable for the desired expression purposes such as, for example, the polypeptide-encoding nucleotide sequence that produces the most protein, produces the most active protein, produces the largest amount of active protein, produces the most stable protein, or other reason provided herein or known in the art.

In one embodiment, the translational kinetics of an mRNA into polypeptide can be changed in order to include a codon pair that inserts or preserve one or more translational pauses and in order to replace at least one codon pair that causes a translational pause with a codon pair that does not cause a translational pause. Methods and criterion for inserting or preserving translational pauses, as well as methods and criterion for removing translational pauses are provided elsewhere herein and can be applied to the present embodiment.

Redesign of Polypeptide-Encoding Nucleotide Sequence

As provided herein, codon pairs are associated with translational pauses, and can thereby influence translational kinetics of an mRNA into polypeptide. Thus, the methods of changing translational kinetics provided herein will typically be performed by modifying or designing one or more nucleotide sequences encoding a polypeptide to be expressed. Accordingly, provided herein are methods of modifying a gene or designing a synthetic nucleotide sequence encoding the polypeptide encoded by the gene, collectively referred to herein as redesigning a polypeptide-encoding gene sequence or redesigning a polypeptide-encoding nucleotide sequence. Also included in the various embodiments provided herein are redesigned gene sequences encoding polypeptides that are not identical to the original gene.

Thus, provided herein are methods for redesigning a polypeptide-encoding nucleotide sequence to modify the translational kinetics of the polypeptide-encoding nucleotide sequence, where the polypeptide-encoding nucleotide sequence is altered such that one or more codon pairs have a decreased likelihood of causing a translational pause or slowing relative to the unaltered polypeptide-encoding nucleotide sequence. For example, one or more nucleotides of a polypeptide-encoding nucleotide sequence can be changed such that a codon pair containing the changed nucleotides has a translational kinetics value indicative of a decreased likelihood of causing a translational pause or slowing relative to the unchanged polypeptide-encoding nucleotide sequence.

While it will be understood by those of skill in the art that a redesigned polypeptide-encoding nucleotide sequence need not possess a high degree of identity to the polypeptide-encoding nucleotide sequence of the original gene, in some embodiments, the redesigned polypeptide-encoding nucleotide sequence will have at least 50%, 60%, 70%, 80%, 90%, 91%, 92%, 93%, 94%, 95%, 96%, 97%, 98% or 99% identity with the polypeptide-encoding nucleotide sequence of the original gene. As used to herein an “original gene” refers to a gene for which codon pair refinement is to be performed; such original genes can be, for example, wild type genes, naturally occurring mutant genes, other mutant genes such as site-directed mutant genes. In other embodiments, the polynucleotide sequence will be completely synthetic, and will bear much lower identity with the original gene, e.g., no more than 90%, 80%, 70%, 60%, 50%, 40%, or lower.

Because of the redundancy of the triplet genetic code it is possible to preserve amino acid sequence coding while redesigning the polypeptide-encoding gene nucleotide sequence. Polypeptide-encoding nucleotide sequences can be redesigned to be convenient to work with and specifically tailored to a particular host and vector system of choice. The resulting sequence can be designed to: (1) reduce or eliminate translational problems caused by inappropriate ribosome pausing, such as those caused by over represented codon pairs or other codon pairs with translational values predictive of a translational pause; (2) have codon usage refined to avoid over-reliance on rare codons; (3) reduce in number or remove particular restriction sites, splice sites, internal Shine-Dalgarno sequences, or other sites that may cause problems in cloning or in interactions with the host organism; or (4) have controlled RNA secondary structure to avoid detrimental translational termination effects, translation initiation effects, or RNA processing, which can arise from, for example, RNA self-hybridization. When a synthetic polypeptide-encoding nucleotide sequence is to be used, this sequence also can be designed to avoid oligonucleotides that mishybridize, resulting in genes that can be assembled from refined oligonucleotides that by thermodynamic necessity only pair up in the desired manner.

In some instances, it is not possible to modify the polypeptide-encoding nucleotide sequence to suitably modify the translational kinetics of the mRNA into polypeptide without modifying the amino acid sequence of the encoded polypeptide. In such instances, an amino acid insertion, deletion or mutation can be introduced to yield a codon pair that is not predicted to cause a translational pause or slowing; or no change is made. In methods in which an amino acid insertion, deletion or mutation is made in order to change translational kinetics, it is preferable to select a change that is predicted not to substantially influence the final three-dimensional structure of the protein and/or the activity of the protein. Such an amino acid mutation can include, for example, a conservative amino acid substitution such as the conservative substitutions shown in Table 1. Such non-identical polypeptides can vary by containing one or more insertions, deletions and/or mutations. Although the nature and degree of change to the polypeptide sequence can vary according to the purpose of the change, typically such a change results in a polypeptide that is at least 50%, 60%, 70%, 80%, 90%, 91%, 92%, 93%, 94%, 95%, 96%, 97%, 98% or 99% identical to the wild type polypeptide sequence.

In some embodiments, redesign of the polypeptide-encoding gene sequence is performed in conjunction with optimization of a plurality of parameters, where one such parameter is codon pair usage. Methods already known in the art for optimizing multiple parameters in synthetic nucleotide sequences can be applied to optimizing the parameters recited in the present claims. Such methods may advantageously include those exemplified in U.S. patent App. Publication No. 2005/0106590, and R. H. Lathrop et al. “Multi-Queue Branch-and-Bound Algorithm for Anytime Optimal Search with Biological Applications” in Proc. Intl. Conf. on Genome Informatics, Tokyo, Dec. 17-19, 2001 pp. 73-82; in Genome Informatics 2001 (Genome Informatics Series No. 12), Universal Academy Press, which are incorporated herein by reference in their entireties. Briefly, in addition to optimizing the various parameters recited herein, an exemplary method for generating a synthetic sequence can also include dividing the desired sequence into a plurality of partially overlapping segments; optimizing the melting temperatures of the overlapping regions of each segment to disfavor hybridization to the overlapping segments which are non-adjacent in the desired sequence; allowing the overlapping regions of single stranded segments which are adjacent to one another in the desired sequence to hybridize to one another under conditions which disfavor hybridization of non-adjacent segments; and filling in, ligating, or repairing the gaps between the overlapping regions, thereby forming a double-stranded DNA with the desired sequence. This process can be performed manually or can be automated, e.g., in a general purpose digital computer. In one embodiment, the search of possible codon assignments is mapped into an anytime branch and bound computerized algorithm developed for biological applications.

Accordingly, provided herein are methods of designing a synthetic nucleotide sequence encoding a desired polypeptide, where the synthetic nucleotide sequence also is designed to have desirable translational kinetics properties, such as the removal of some or all codon pairs predicted to result in a translational pause or slowing. Such design methods include determining a set of partially overlapping segments with optimized melting temperatures, and determining the translational kinetics of the synthetic sequence, where if it is desired to change the translational kinetics of the synthetic gene, the sequences of the overlapping segments are modified and refined in order to approximate the desired translational kinetics while still possessing acceptable hybridization properties. In some embodiments, this process is performed iteratively. In some embodiments, a criterion is established for selecting codon pairs having high translational kinetics values to be replaced with codon pairs having lower the translational kinetics values unless a codon pair of this group is the site of a planned pause. For example, the top 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of codon pairs ranked by translational kinetics values can be replaced by codon pairs having lower translational kinetics values, such as translational kinetics value below a user defined level that can be, for example, a translational kinetics value equal to or below the translational kinetics values of codon pairs not in the top selected percentage, unless a codon pair of this group is the site of a planned pause (in which case it is not necessarily replaced). In another example all codon pairs above a user-selected translational kinetics value, such as more than 5, 4.5, 4, 3.5, 3, 2.5 or 2 standard deviations above the mean translational kinetics value can be replaced by codon pairs having lower translational kinetics values, such as translational kinetics value below a user defined level that can be, for example, a translational kinetics value that is 4, 3.5, 3, 2.5, 2, 1.5 or 1 standard deviations less than the mean translational kinetics value, unless a codon pair of this group is the site of a planned pause (in which case it is not necessarily replaced). In some embodiments, graphical displays of values of observed versus expected codon pair frequencies are generated for the original sequence, the final sequence, and/or any intermediate sequences. In other embodiments, graphical displays of refined, possible, or improved translational kinetics values of codon pairs are generated for the original sequence, the final sequence, and/or any intermediate sequences. Such graphical displays can be used for analyzing the translational kinetics of the synthetic nucleotide sequence. Further synthetic nucleotide sequence refinement methods can be employed where additional properties of the synthetic nucleotide sequence can be refined in addition to hybridization and codon pair usage properties, where such properties can include, for example, codon usage, reduced number of restriction sites or Shine-Dalgarno sequences, or reduced detrimental RNA secondary structure, as described above.

Those skilled in the art will recognize that various optimization methods can be used, e.g., simulated annealing, genetic algorithms, branch and bound techniques, hill climbing, Monte Carlo methods, other search strategies, and the like. Thus, the methods provided herein for redesigning the polypeptide-encoding gene sequence that include optimization of a plurality of parameters, where one such parameter is codon pair usage, can be implemented in by applying those parameters to art-recognized algorithms or techniques. Advantageously, redesign of the polypeptide-encoding gene sequence is performed using an optimization method that designs a synthetic nucleotide sequence encoding the polypeptide to be expressed.

The polypeptide-encoding nucleotide sequence redesign methods provided herein can be employed where a plurality of properties of the polypeptide-encoding nucleotide sequence can be refined in addition to codon pair usage properties, where such properties can include, but are not limited to, melting temperature gap between oligonucleotides of synthetic gene, average codon usage, average codon pair chi-squared (e.g., z score), worst codon usage, worst codon pair (e.g., z score), maximum usage in adjacent codons, Shine-Dalgarno sequence (for E. coli expression), occurrences of 5 consecutive G's or 5 consecutive C's, occurrences of 6 consecutive A's or 6 consecutive T's, long exactly repeated subsequences, cloning restriction sites, user-prohibited sequences (e.g., other restriction sites), codon usage of a specific codon above user-specified limit, and out-of-frame stop codons (framecatchers). In embodiments that include expression in a eukaryotic host organism, additional properties that can be considered in a process of redesigning a polypeptide-encoding nucleotide sequence include, but are not limited to, occurrences of RNA splice sites, occurrences of polyA sites, and occurrence of Kozak translation initiation sequence. For example, a process of redesigning a polypeptide encoding nucleotide sequence can include constraints including, but not limited to, minimum melting temperature gap between oligonucleotides of synthetic gene, minimum average codon usage, maximum average codon pair chi-squared (z score), minimum absolute codon usage, maximum absolute codon pair (z score), minimum maximum usage in adjacent codons, no Shine-Dalgarno sequence (for E. coli expression), no occurrences of 5 consecutive G's or 5 consecutive C's, no occurrences of 6 consecutive A's or 6 consecutive T's no long exactly repeated subsequences, no cloning restriction sites, no user-prohibited sequences (e.g., other restriction sites), and optionally no codon usage of a specific codon above user-specified limit. In embodiments that include expression in a eukaryotic host organism, additional constraints can include, but are not limited to, minimum occurrences of RNA splice sites, minimum occurrences of polyA sites, and occurrence of Kozak translation initiation sequence. A process of redesigning a polypeptide-encoding nucleotide sequence can include preferences including, but not limited to, prefer high average codon usage, prefer low average codon pair chi-squared, prefer larger melting temperature gap, prefer more out of frame stop codons (framecatchers), and optionally prefer evenly distributed codon usage. Any of a variety of nucleotide sequence refinement/optimization methods known in the art can be used to refine the polypeptide-encoding nucleotide sequence according to the codon pair usage properties, and according to any of the additional properties specifically described above, or other properties that are refined in nucleotide sequence redesign methods known in the art. In some embodiments, a branch and bound method is employed to refine the polypeptide-encoding nucleotide sequence according to codon pair usage properties and at least one additional property, such as codon usage.

In some embodiments, the methods provided herein can further include analyzing at least a portion of the candidate polynucleotide sequence in frame shift, and selecting codons for the candidate polynucleotide sequence such that stop codons are added to at least one said frame shift. In additional embodiments, the generating step further includes analyzing at least a portion of the candidate polynucleotide sequence in frame shift, and selecting codons for the candidate polynucleotide sequence such that one or more stop codons in one, two or three reading frames are added downstream of polypeptide-encoding region of the nucleotide sequence.

In some embodiments, methods are provided for redesigning a polypeptide-encoding gene for expression in a host organism, by providing a data set representative of codon pair translational kinetics for the host organism which includes translational kinetics values of the codon pairs utilized by the host organism, providing a desired polypeptide sequence for expression in the host organism, and generating a polynucleotide sequence encoding the polypeptide sequence by analyzing candidate nucleotides to select, where possible, codon pairs that are predicted not to cause a translational pause in the host organism, with reference to the data set, thereby providing a candidate polynucleotide sequence encoding the desired polypeptide.

Also provided herein are methods for redesigning a polypeptide-encoding gene for expression in a host organism, by providing a first data set representative of codon pair translational kinetics for the host organism which includes translational kinetics values of the codon pairs utilized by the host organism, providing a second data set representative of at least one additional desired property of the synthetic gene, providing a desired polypeptide sequence for expression in the host organism, and generating a polynucleotide sequence encoding the polypeptide sequence by analyzing candidate nucleotides to select, where possible, both (i) codon pairs that are predicted not to cause a translational pause in the host organism, with reference to the first data set, and (ii) nucleotides that provide a desired property, with reference to the second data set, thereby providing a candidate polynucleotide sequence encoding the desired polypeptide. In some embodiments, a branch and bound method is employed to refine the polypeptide-encoding nucleotide sequence according to codon pair usage properties of the first data set and according to the properties of the second data set. In some embodiments, the second data set contains of codon preferences representative of codon usage by the host organism, including the most common codons used by the host organism for a given amino acid.

The methods provided herein can further include analyzing the candidate polynucleotide sequence to confirm that no codon pairs are predicted to cause a translational pause in the host organism by more than a designated threshold. As described elsewhere herein, the likelihood that a particular codon pair will cause translational pausing or slowing in an organism (or the relative predicted magnitude thereof) can be represented by a translational kinetics value. The translational kinetics value can be expressed in any of a variety of manners in accordance with the guidance provided herein. In one example, a translational kinetics value can be expressed in terms of the mean translational kinetics value and the corresponding standard deviation for all codon pairs in an organism. For example, the translational kinetics value for a particular codon pair can be expressed in terms of the number of standard deviations that separate the translational kinetics value of the codon pair from the mean translational kinetics value. In methods that include analyzing the candidate polynucleotide sequence to confirm that no codon pairs are predicted to cause a translational pause in the host organism by more than a designated threshold, a threshold value can be at least 1, 1.25, 1.5, 1.75, 2, 2.25, 2.5, 3, 3.5, 4, 4.5 or 5 or more standard deviations above the mean translational kinetics value. Although such a method is described in terms of a binary scoring of a codon pair as either at least or less than the threshold value, one skilled in the art, in view of the teachings herein, will recognize that multiple thresholds can be used, or methods can be used that weight a codon pair along a continuum according to the translational kinetics value, based on the teachings provided herein and the general knowledge in the art.

In some embodiments, in addition to generating a candidate nucleotide sequence according to codon pair usage properties, the methods provided herein also include generating a candidate nucleotide sequence according to codon usage. As is known in the art, different organisms can have different preference for the three-nucleotide codon sequence encoding a particular amino acid. As a result, translation can often be improved by using the most common three-nucleotide codon sequence encoding a particular amino acid. Thus, some methods provided herein also include generating a candidate nucleotide sequence such that codon utilization is non-randomly biased in favor of codons most commonly used by the host organism. Codon usage preferences are known in the art for a variety of organisms and methods for selecting the more commonly used codons are well known in the art.

In some embodiments, the methods of redesigning a polypeptide-encoding nucleotide sequence are based on a plurality of properties, where a conflict in the preferred nucleotide sequence arising from the plurality of properties is determined in order to optimize the predicted translational kinetics. That is, when the plurality of properties being optimized would lead to more than one possible nucleotide sequence depending on which property is to be accorded more weight, typically, the conflict is resolved by selecting the nucleotide sequence predicted to be translated more rapidly, for example, due to fewer predicted translational pauses. In some embodiments, the methods of redesigning a polypeptide-encoding nucleotide sequence are based on a plurality of properties, where a conflict in the preferred nucleotide sequence arising from the plurality of properties is determined in order to optimize codon pair usage preferences. That is, when the plurality of properties being optimized would lead to more than one possible nucleotide sequence depending on which property is to be accorded more weight, typically, codon pair usage will be accorded more weight in order to resolve the conflict between the more than one possible nucleotide sequences. In one example, the methods provided herein can include identifying at least one instance of a conflict between selecting common codons and avoiding codon pairs predicted to cause a translational pause; in such instances, the conflict is resolved in favor of avoiding codon pairs predicted to cause a translational pause.

Some embodiments provided herein include generating a candidate polynucleotide sequence encoding the polypeptide sequence, the candidate polynucleotide sequence having a non-random codon pair usage, such that the codon pairs encoding any particular pair of amino acids have the lowest translational kinetics values. In some embodiments, the candidate polynucleotide sequence encoding the polypeptide sequence is generated and/or altered such that the encoded amino acid sequence is not altered. In some embodiments, the candidate polynucleotide sequence encoding the polypeptide sequence is generated and/or altered such that the three dimensional structure of the encoded polypeptide is not substantially altered. In some embodiments, the candidate polynucleotide sequence encoding the polypeptide sequence is generated and/or altered such that no more than conservative amino acid changes are made to the encoded polypeptide.

The methods provided herein can further include a step of refining or altering the candidate polynucleotide sequence in accordance with a second nucleotide sequence property to be refined. For example, in embodiments in which codon usage is also refined, the methods further include generating or refining a candidate polynucleotide sequence encoding a polypeptide sequence such that the candidate polynucleotide sequence has a non-random codon usage, where the most common codons used by the host organism are over-represented in the candidate polynucleotide sequence. The methods can include refining or altering the candidate polynucleotide sequence in accordance with any of a variety of additional properties provided herein, including but not limited to, melting temperature gap between oligonucleotides of synthetic gene, Shine-Dalgarno sequence, occurrences of 5 consecutive G's or 5 consecutive C's, occurrences of 6 consecutive A's or 6 consecutive T's long exactly repeated subsequences, cloning restriction sites, or any other user-prohibited sequences. Further, any of a variety of combinations of these properties can be additionally included in the nucleotide sequence refinement methods provided herein.

The method provided herein can further include an evaluation step in which after the candidate polynucleotide sequence is altered, the sequence is compared with at least a portion of a data set of a property against which the sequence was refined. In such methods, it is possible to compare the candidate sequence to the data set in order to determine whether or not the candidate sequence possesses the desired or acceptable properties with respect to the data set. For example, subsequent to a round of nucleotide sequence refinement, it can be evaluated whether or not the codon pairs of the candidate sequence have acceptable translational kinetics values. If the values are deemed to be acceptable or desired, no further sequence alteration is required with respect to the property. In view of the methods provided herein which can be directed to the refinement or optimization of a plurality of properties, the candidate nucleotide sequence can be compared to each property considered in the refinement, and, if the values for all properties are deemed to be acceptable or desired, no further sequence alteration is required. If the values for fewer than all properties are deemed to be acceptable or desired, the candidate nucleotide sequence can be subjected to further sequence alteration and evaluation.

Thus, it is contemplated herein that the sequence alteration steps of methods provided herein can be performed iteratively. That is, one or more steps of altering the nucleotide sequence can be performed, and the candidate nucleotide sequence can be evaluated to determine whether or not further sequence alteration is necessary and/or desirable. These steps can be repeated until values for all properties are deemed to be acceptable or desired, or until no further improvement can be achieved.

The graphical displays and methods provided herein can be used in a variety of applications provided herein, and additional applications that will be readily apparent to one skilled in the art. For example, the graphical displays and methods provided herein can be used in methods of genetic engineering, in development of biologics such as therapeutic biologics, preparation of immunological reagents including vaccines, preparation of serological diagnostic products, and additional protein production technologies known in the art.

In addition to the specifically recited steps in all methods provided herein, an additional step can include outputting the results of the method, where the output can be to a computer-readable medium such as a fixed computer-readable medium or a transient computer-readable medium, or the output can be to a user-readable form such as a paper printout or a display on a computer monitor.

The methods described herein are typically implemented on one or more computing devices, optionally in a computer network environment. A computing device suitable for practicing various aspects of the methods disclosed herein is provided. The computer device may take various forms. In one embodiment, the computing device is a personal computer such as a supercomputer, clustered computers, a desktop computer or a laptop computer.

The computer device typically includes many operating components, several of which are shown here. The computing device includes one or more processors. The processor may be a central processing unit which is configured to interpret computer program instructions and process data. Well known examples of central processing units are chips offered by Intel® and Advanced Micro Devices, Inc. which are typically installed in desktop computers. The computing device may also include a volatile memory such as random access memory (RAM). The computing device may further include non-volatile memory. The non-volatile memory may take various forms. The non-volatile memory may include a hard disk drive or some other type of mass storage media. The non-volatile memory may further include flash memory, or some form of read only memory (ROM) such as a PROM, EPROM, or EEPROM.

Stored on the non-volatile memory may be an operating system. The operating system may be a well known computer desktop operating system such as Windows®, MacOS®, Unix or Linux. Also stored on the non-volatile memory may be application software. The application software typically includes end user software applications such as web browsers, business applications and the like. In some embodiments, the systems and methods described herein are implemented as application software programs running within or on top of the operating system. In other embodiments, the knowledge acquisition systems described below may be implemented as a web-based application running within a web browser. Also included in the non-volatile memory may be application data. A portion of the application data may be data that is related to the knowledge acquisition systems described in further detail below. In particular, the application data 110 may include “electronic flashcard” data, graphical data, audio data, or some other data.

The computing device also includes one or more input devices which are used to input data into the computing device by the user. The input devices may include a keyboard, a mouse, a stylus, a touch screen, input a microphone, joystick, game pad, satellite dish, scanner, or the like. The computing device also includes a display. The display typically provides a graphical user interface with which a user may interact to control the operation of the computing device.

As certain embodiments may be implemented in a computer network environment, the computing device may be equipped with a network interface. The network interface may take the form of a network interface card (NIC) which may provide the computing device with the ability to communicate with other computers on the network. The NIC may be a wireless network card, a wired network card, or both. The computing device may further include a removable storage media. The removable storage media may take the form of a memory stick, a writeable CD or DVD, a floppy disk, or some other storage media. The removable storage media may be used to store application data and to transfer application data between computing devices. The removable storage media also may be used to store results generated by the application, such as, for example, translational kinetics values.

Also provided herein is a computer usable medium having computer readable program code embodied therein for calculating translational kinetics values, the computer readable code comprising instructions for determining translational kinetics values according to any one of the various methods provided herein elsewhere. Also provided herein is a computer usable medium having computer readable program code embodied therein for modifying a polypeptide-encoding nucleotide sequence, the computer readable code comprising instructions for modifying a polypeptide-encoding nucleotide sequence according to any one of the various methods provided herein elsewhere. Also provided herein is a computer usable medium having computer readable program code embodied therein for redesigning a polypeptide-encoding nucleotide sequence, the computer readable code comprising instructions for redesigning a polypeptide-encoding nucleotide sequence according to any one of the various methods provided herein elsewhere. Also provided herein is a computer usable medium having computer readable program code embodied therein for graphically displaying the translation kinetics of a polypeptide-encoding nucleotide sequence, the computer readable code comprising instructions for graphically displaying the translation kinetics of a polypeptide-encoding nucleotide sequence according to any one of the various methods provided herein elsewhere.

Also provided herein is a computer readable medium containing software that, when executed, causes the computer to perform the acts of determining translational kinetics values according to any one of the various methods provided herein elsewhere. Also provided herein is a computer readable medium containing software that, when executed, causes the computer to perform the acts of modifying a polypeptide-encoding nucleotide sequence according to any one of the various methods provided herein elsewhere. Also provided herein is a computer readable medium containing software that, when executed, causes the computer to perform the acts of redesigning a polypeptide-encoding nucleotide sequence according to any one of the various methods provided herein elsewhere. Also provided herein is a computer readable medium containing software that, when executed, causes the computer to perform the acts of graphically displaying the translation kinetics of a polypeptide-encoding nucleotide sequence according to any one of the various methods provided herein elsewhere.

The following examples are included for illustrative purposes only and are not intended to limit the scope of the invention.

### Example 1

This example describes graphical displays of z scores for expression of a gene from a yeast retrotransposon in yeast and bacteria, and E. coli expression levels of different nucleotide sequences encoding the same protein. Ty3 is a retrotransposon of Saccharomyces cerevisiae, and is adapted to express its genes in S. cerevisiae using S. cerevisiae translational machinery. Thus, expression of Ty3 genes in S. cerevisiae represents native expression of these genes.

Chi-squared values for S. cerevisiae and E. coli were determined using previously reported methods (Hatfield and Gutman, “Codon Pair Utilization Bias in Bacteria, Yeast, and Mammals” in Transfer RNA in Protein Synthesis, Hatfield, Lee and Pirtle Eds. CRC Press (Boca Raton, La.) 1993). Briefly, nonredundant protein coding regions for each organism was obtained from GenBank sequence database (75,403 codon pairs in 177 sequences for S. cerevisiae, and 75,096 codon pairs in 237 sequences for E. coli) to determine an observed number of occurrences for each codon pair. The expected number of occurrences of each codon pair was calculated under the assumption that the codon pairs are used randomly. The chi-squared value “chisq1” was generated by the expected and observed values determined. The chsq1 was re-calculated to remove any influence of non-randomness in amino acid pair frequencies, yielding “chisq2.” The chsq2 was re-calculated to remove any influence of non-randomness in dinucleotide frequencies, yielding “chisq3.” z scores of chisq3 were calculated by determining the mean chisq3 value and corresponding standard deviation for all codon pairs, and normalizing each chisq3 value to be reported in terms of number of standard deviations from the mean chisq3 values.

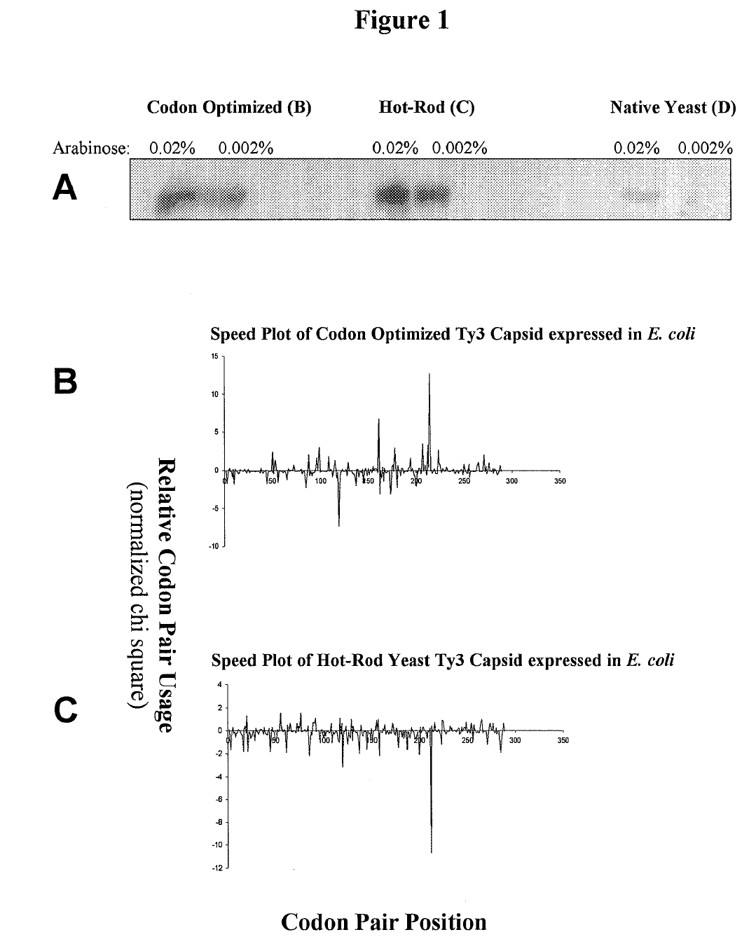

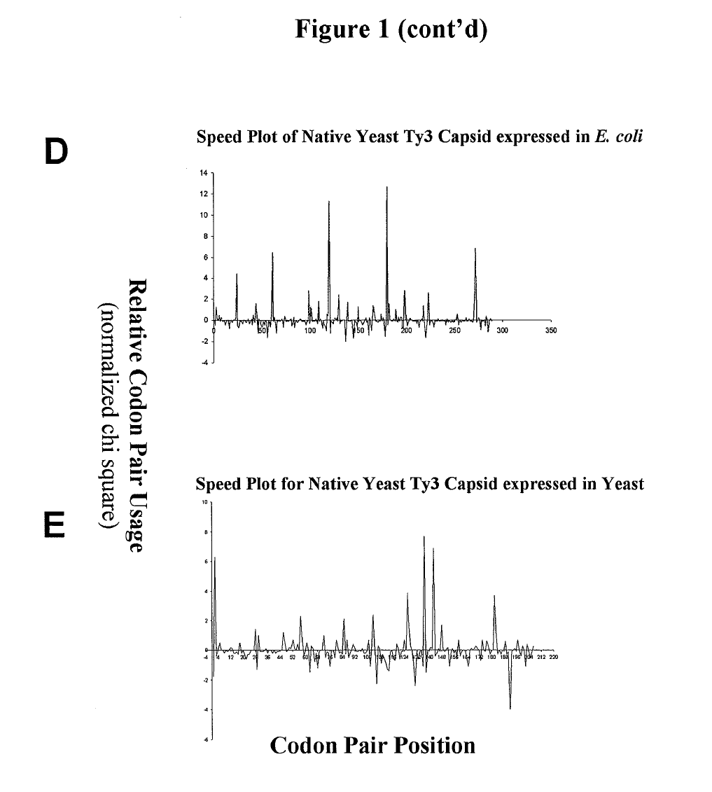

The nucleotide sequence for the gene encoding the Ty3 capsid protein was modified to optimize codon usage. A graphical display for the codon usage optimized gene (SEQ ID NO:3) encoding the Ty3 capsid protein (SEQ ID NO:4) expressed in E. coli was prepared by plotting z scores of chi-squared values for codon pair utilization in E. coli as a function of codon pair position. The graphical display is provided in FIG. 1B.

The nucleotide sequence for the gene encoding the Ty3 capsid protein was modified to no longer contain codon pairs having z scores in E. coli greater than 2. A graphical display for the codon pair utilization-modified gene (SEQ ID NO:5) encoding the Ty3 caspid protein (SEQ ID NO:6) expressed in E. coli was prepared by plotting z scores of chi-squared values for codon pair utilization in E. coli as a function of codon pair position. The graphical display is provided in FIG. 1C.

A graphical display for the native gene (SEQ ID NO:1) encoding the Ty3 caspid protein (SEQ ID NO:2) expressed in E. coli was prepared by plotting z scores of chi-squared values for codon pair utilization in E. coli as a function of codon pair position. The position. The graphical display is provided in FIG. 1D.

A graphical display for the native gene (SEQ ID NO:1) encoding the Ty3 capsid protein (SEQ ID NO:2) in S. cerevisiae was prepared by plotting z scores of chi-squared values for codon pair utililization in S. cerevisiae as a function of codon pair position. The graphical display is provided in FIG. 1E.

Expression in E. coli of the codon optimized, codon pair utilization-based modification (hot-rod) and native Ty3 capsid was examined by Western blot analysis. pBAD-GAG was transformed into E. coli strain Top 10 (F-mcrA delta(mrr-hsdRMS-mcrBC) phi 80lacZ deltaM15 deltalacX74 deoR recA1 araD139 delta(ara-leu) 7697 galU galK rpsL (StrR) endA1nupG). An overnight culture was inoculated at 1:100 into 5 ml of LB medium plus 100 μg/ml ampicillin and grown at 37° C. to OD₆₀₀ of 0.5. Protein expression was induced by addition of 0.002 or 0.02% L-arabinose and grown for 3 hrs at 37° C. Cells were harvested by centrifugation and the cell pellet was resuspended in phosphate buffered saline. Cells were disrupted by sonication and supernatant and pellet fractions were resolved in a 4-20% SDS-polyacrylamide gel (Pierce). Proteins were transferred to Immobilon-P (Millipore, Bedford, Mass.) and were incubated with rabbit polyclonal anti-Ty3 CA (capsid) antibody diluted 1:20,000. Rabbit IgG was visualized using a HRP-conjugated secondary antibody and ECL+Plus (Amersham, Buckinghamshire, UK) according to manufacturer's instructions. The results of the Western blot analysis are provided in FIG. 1A.

FIG. 1A demonstrates that changes to a polypeptide-encoding nucleic acid sequence can increase expression of the polypeptide, particularly when the polypeptide is heterologously expressed. Specifically, FIG. 1A shows that the unmodified Ty3 capsid encoding nucleic acid sequence yields low levels of Ty3 capsid expression in E. coli. In contrast, a codon optimized Ty3 capsid-encoding nucleic acid sequence yields high levels of Ty3 capsid expression in E. coli, and codon pair utilization-based modified Ty3 capsid encoding nucleic acid sequence yields the highest levels of Ty3 capsid expression in E. coli. Further demonstrated in FIG. 1 is the influence of the location in the polypeptide-encoding nucleotide sequence of an over-represented codon pair on the expression levels of the protein. FIG. 1D, corresponding to the lowest expression levels of Ty3 capsid, depicts two predicted pause sites within the first 70 codons. In contrast, FIG. 1B and FIG. 1E both depict predicted pause sites, but these pause sites are further downstream relative to the pause sites in FIG. 1D (note that although not depicted, Ty3 capsid is known to be expressed at high levels in S. cerevisiae). These results demonstrate that over-represented codon pairs closer to the amino terminus/translation initiation site can have a stronger influence on protein expression levels compared to over-represented codon pairs situated further downstream (i.e., closer to the carboxy terminus).

### Example 2

This example describes the use of graphical displays of codon pair usage versus codon pair position in conjunction with knowledge of the secondary and tertiary structure of a polypeptide in evaluating over-represented codon pairs and the importance of pause sites between protein structural elements.

Normalized chi-squared values of codon pair utililization were plotted versus codon pair position for nucleic acid sequences encoding the capsid protein of the human immunodeficiency virus, HIV-1, and the capsid protein of the S. cereviseae retrotransposon, Ty3. The three-dimensional structure of the HIV-1 capsid protein has been determined experimentally, and the structural elements of the Ty3 capsid protein have been predicted by conventional threading methods to be similar to those of the HIV-1 capsid protein. The ribbon structure depicting alpha helices of each protein is shown above the respective graphical display. The regions of the abscissa indicating the amino terminal and the carboxy terminal domains of each protein are indicated by brackets. The thick black horizontal lines aligned in parallel with the codon pair position identify the positions of each alpha helix in each protein.

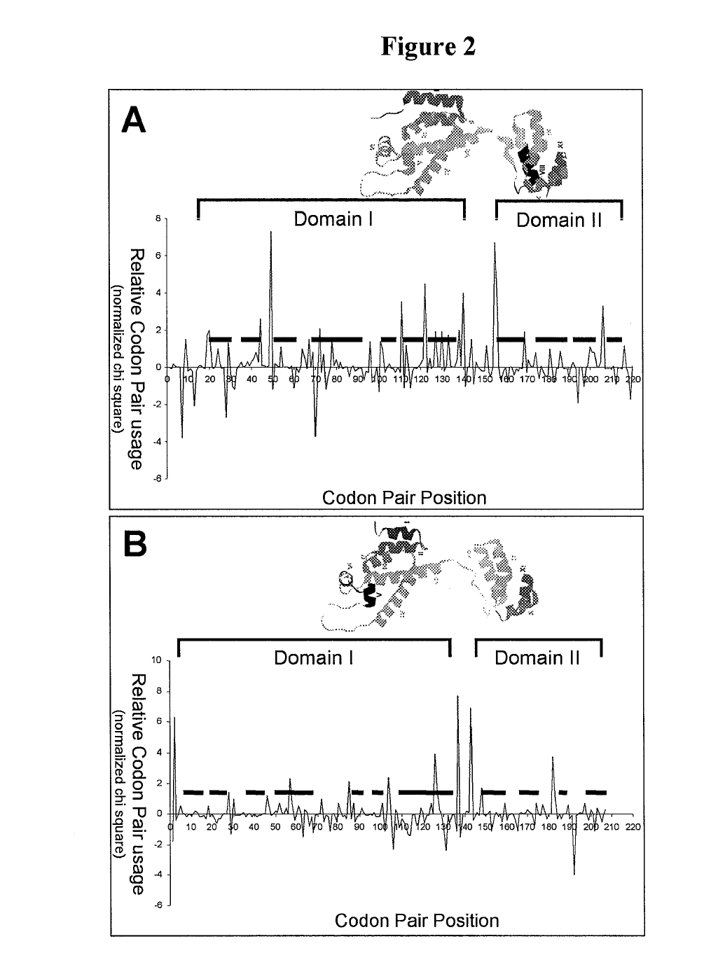

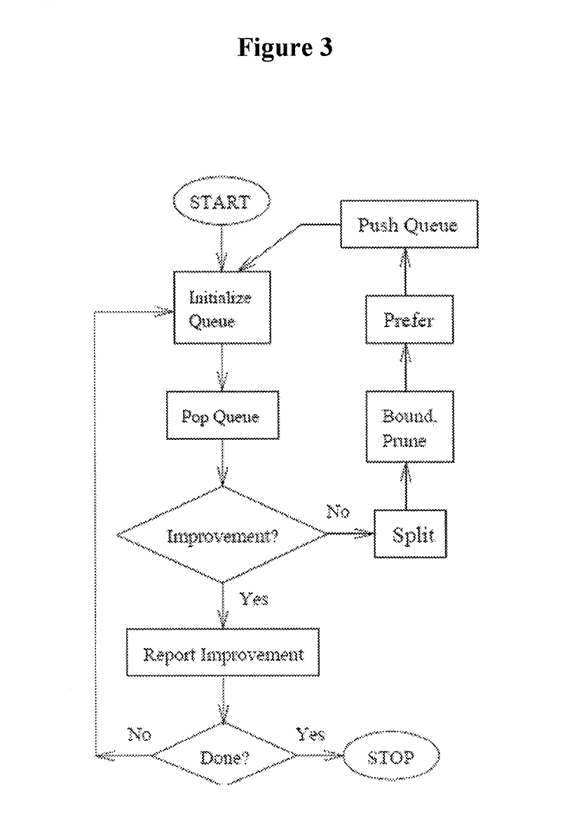

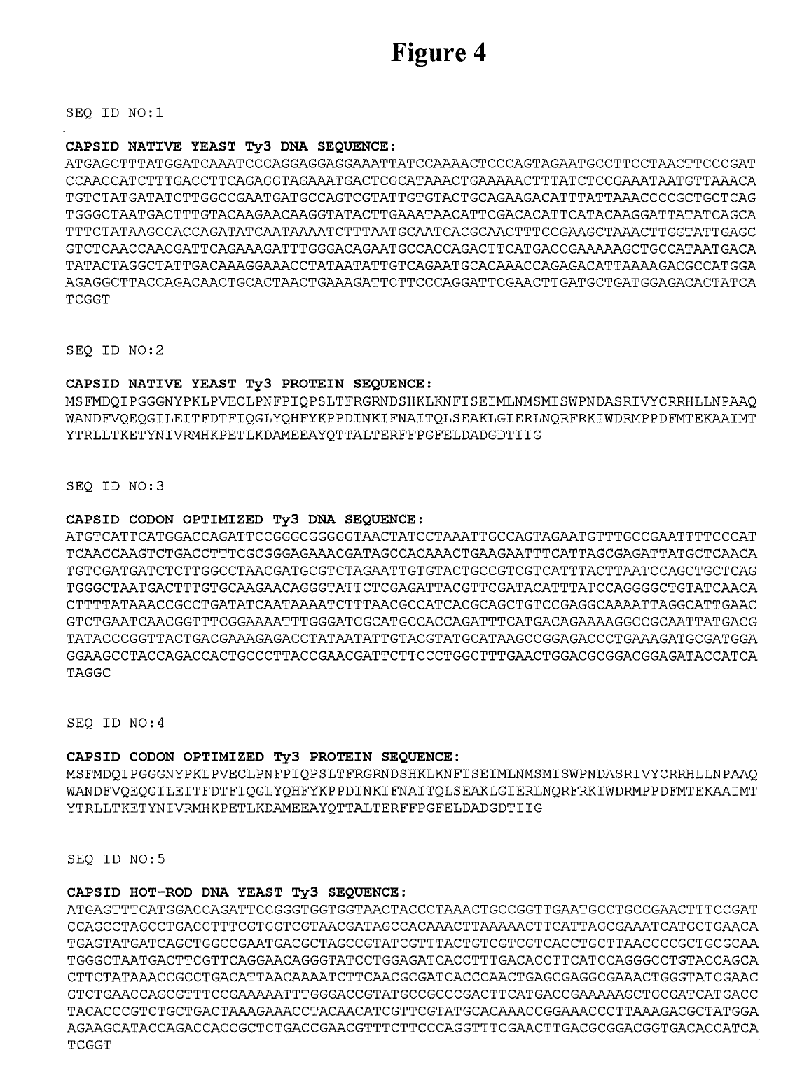

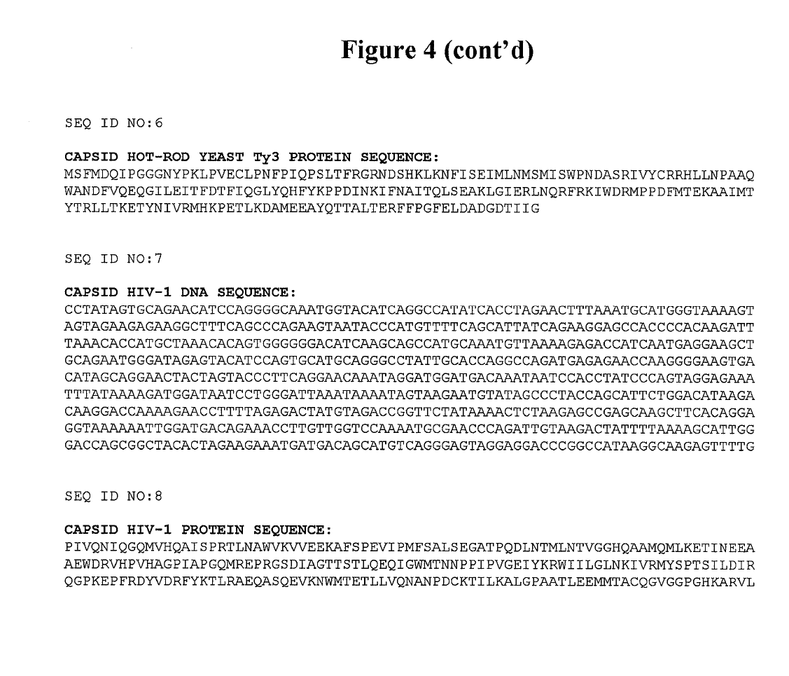

The plot of codon pair utililization versus codon pair position for the gene (SEQ ID NO:7) encoding the HIV-1 capsid protein (SEQ ID NO:8) expression in human is provided in FIG. 2A. In this figure, codon pairs having normalized chi-squared values greater than approximately 2 are not present in regions encoding amino acids located within an alpha helix, but are present in regions encoding amino acids located between alpha helices, and in particular, are present in regions immediately N-terminal to, or immediately C-terminal to, an alpha helix. In addition, two highly over-represented codon pairs are located between the N-terminal and C-terminal domains, and, in particular, the first is located immediately C-terminal to the N-terminal domain and the second is located immediately N-terminal to the C-terminal domain.

The plot of codon pair utililization versus codon pair position for the native gene (SEQ ID NO: 1) encoding the Ty3 capsid protein (SEQ ID NO:2) expressed in S. cerevisiae is provided in FIG. 2B. Protein amino acid similarity between Ty3 and HIV-1 capsid protein is 16.6%, and DNA sequence similarity between Ty3 and HIV-1 capsid protein is considered to be even lower than 16.6%. Despite the lack of sequence similarity, FIG. 2B shows several similarities with FIG. 2A. Specifically, except for two instances, codon pairs having normalized chi-squared values greater than approximately 2 are not present in regions encoding amino acids located within an alpha helix. Further, numerous codon pairs having normalized chi-squared values greater than approximately 2 are present in regions between alpha helices, and in particular, are present in regions immediately N-terminal to, or immediately C-terminal to, an alpha helix. In addition, two highly over represented codon pairs are located between the N-terminal and C-terminal domains, and, in particular, the one such codon pair is located immediately C-terminal to the N-terminal domain.

These plots demonstrate that it is possible to use graphical displays of translational kinetics to validate or obtain evidence confirming the likelihood that an over represented codon pair indicates a translational pause site. These plots also demonstrate that it is possible to analyze polypeptide-encoding nucleotide sequences of structurally similar proteins from different species in order to validate or obtain evidence confirming the likelihood that an over-represented codon pair indicates a translational pause site, or validate or obtain evidence confirming the likelihood that a particular site in the sequence contains a translational pause. These plots also demonstrate that it is possible to analyze polypeptide-encoding nucleotide sequences in conjunction with the secondary and/or tertiary structure of the polypeptide in order to validate or obtain evidence confirming the likelihood that an over-represented codon pair indicates a translational pause site, or validate or obtain evidence confirming the likelihood that a particular site in the sequence contains a translational pause.

### Example 3

This example describes creation of generic translational kinetics values.

Generic species datasets can be generated by following the hierarchy of the phylogenetic tree of life. Starting at the root of the tree, each mid-level node of the phylogenetic tree, which could be a family, genus, or higher level, represents a collection of all the species in the sub-tree under this node, until the tree reaches the lowest level nodes, which correspond to individual species.

For example, in order to create a generic set of translational kinetics values, such as generic mammal, genomic sequences from various mammalian species such as human (Homo sapiens), monkey (Macaca mulatta, Macaca fascicularis), chimpanzee (Pan troglodytes), sheep (Ovis aries), dog (Canis familiaris), and cow (Bos Taurus) can be pooled. In another example, a generic rodent dataset can include genomic sequences from rat (Rattus novegicus), mouse (Mus musculus), and Chinese hamster (Cricetulus griseus).

In one exemplary embodiment, in order to create a generic dataset of the Saccharomycetaceae family, sequences from such species as Saccharomyces bayanus, Saccharomyces castellii, Saccharomyces kluyveri, Saccharomyces kudriavzevii, Saccharomyces mikatae, Saccharomyces paradoxus, Pichia stipitis, Pichia pastoris, Pichia minuate, and Debaryomyces hansenii, etc., can all be included. These species are all part of the Saccharomycetaceae family.

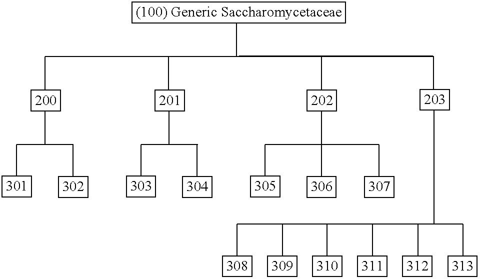

100 Generic Saccharomycetaceae

200 Generic Debaryomyces

201 Generic Kluyveromyces

202 Generic Pichia

203 Generic Saccharomyces

301 Debaryomyces hansenii

302 Debaryomyces occidentalis

303 Kluyveromyces lactis

304 Kluyveromyces waltii

305 Pichia pastoris

306 Pichia stipitis

307 Pichia minuta

308 Saccharomyces bayanus

309 Saccharomyces castellii

310 Saccharomyces kluyveri

311 Saccharomyces kudriavzevii

312 Saccharomyces mikatae

313 Saccharomyces paradoxus

The first step to generating a generic codon pair dataset is to gather all the coding region sequences of all the genes in the nodes (e.g., species) included in the sub-tree, to the extent that the sequences are available.

Generic species datasets can be created at any level of the phylogenetic tree except at the lowest (e.g., species or leaf nodes) level. In an exemplary embodiment, a collection of nodes (for example, 305, 306 and 307) can be clustered and formed into a group. This new group becomes a generic dataset for the nodes it includes; for example, a generic Pichia dataset can be formed.

After all the sequences within the range of the generic datasets are collected, and sequence redundancy is reduced, codon pair statistics can be calculated based on these sequences. Sometimes not all sequences from the member species are included in the generic dataset; for example, if there are any data quality problems, if a sequence's coding region contains uncertain base codes such as N, or if stop codons are found anywhere besides the end of the sequence, then the sequence may not be included in the constructed generic dataset.

### Example 4

This example describes creation of generic translational kinetics values.

Most datasets retrieved from public databases such as Genbank and EMBL contain redundant sequences as a result of repeated or similar research efforts, natural gene duplication, and other reasons. Some of these sequences are complete repeats of other sequences, and some sequences have very high sequence similarities among themselves. If the repeated or highly similar sequences were to be included directly in the dataset and then submitted for statistical calculations, the results can be highly biased. As a result, the redundancy in the datasets is typically eliminated before it is submitted for statistics calculations.

However, removing data redundancy by simply deleting sequences that are highly redundant can also result in the exclusion of certain codon pairs. For example, if a sequence is 90% similar to another sequence and is marked for elimination, the remaining 10% that is different could contain codon pair patterns that are not represented in other sequences. In order to both get rid of data redundancy and keep the variety of codon pairs included in each sequence, similar genomic sequences can be grouped into clusters before statistical analysis. The number of occurrences of each codon pair in sequences within a cluster is divided by the total number of sequences in the group, referred to as the redundancy index.

This can be implemented by starting with the first sequence in a dataset, since this is the first sequence, a cluster called cluster A is made for it, and sequence 1 is the only member in cluster A. Then the second sequence in the dataset is considered. If sequence 2 is similar to sequence 1 using the standard given by the user (such as E value<0.1, or 0.01, or 0.001, etc.), then sequence 2 is also assigned to be a member of cluster A. Because cluster A now has two members, the redundancy index of cluster A is increased to 2. However, if sequence 2 is not similar to sequence 1, then sequence 2 is not a member of cluster A, and new cluster called cluster B is started for sequence 2, and the redundancy of cluster A and B are both 1.

As each sequence in the dataset is scanned for similarity, if it is found to be similar to a known sequence, it is added to the cluster of the known sequence, and the redundancy index value of all the members in the cluster is increased by 1. If the sequence scanned is not similar to any other sequences that have been processed, a new cluster is started for it, and as with all the new clusters, the redundancy index is initiated to 1.

The final output contains each sequence in the dataset together with its redundancy index. By the time this program stops, all the sequences are assigned a redundancy index number, and all the sequences belong to their corresponding clusters All members of the same cluster should have the same redundancy index number. To process this output, the chi-squared values can be calculated by counting the number of occurrences of each codon pair in the sequence dataset and recording the redundancy index of each sequence. In performing the chi-squared calculation, when a codon pair is observed, instead of adding 1 directly to the number of total occurrences of this particular codon pair, the reciprocal of the redundancy index is added instead.

### Example 5

This example describes creation of generic translational kinetics values as in Example 3 by pre-processing the sequence data according to Example 4.

If the sequences included in the species datasets are taken directly into building a generic dataset without pre-processing, noise can be introduced and can affect the quality of the generic datasets generated by this method. One way to reduce noise is to combine the generic species dataset approach of Example 3 and the clustering similar sequences approach of Example 4. For each member dataset, a clustered sequence redundancy reduction is performed as described in Example 4. The weighted sequences are then included in the generic species sequence dataset as described in Example 3. The clustering step not only reduces data redundancy, but also preserves the sequence variety in genomic sequences.

### Example 6

This example describes empirical measurement of codon pair translational step times.

Gene Preparation and Phage Construction

The lacZ gene from Escherichia coli is modified to have all predicted translational pauses removed for expression in E. coli. The modified lacZ gene is transformed into the E. coli lacZ strain MC4100, which is then infected with ?RS88 (Simons et al., Gene (1987) 53:85-96) generating a new bacteriophage lambda containing the modified lacZ. The new bacteriophage lambda is then used to generate monolysogens in the unique attB site in the E. coli chromosome of strain MC4100.

Codon Pair Mutation

The lacZ gene is mutated using site-directed mutagenesis to alter the codon pairs at positions 3/4 or at positions 14/15. Each of these altered lacZ genes is then used for creating novel lambda phage lysates and monolysogens according to the above.

Step Times Measurements

β-galactosidase measurements are taken for each monolysogen strain by measuring the rate of ONPG hydrolysis according to known methods (Miller, J. 1972. Experiments in Molecular Genetics, p. 352-355. Cold Spring Harbor Laboratory, NY.). β-galactosidase activities are measured using a TECAN GENiosPlus microplate reader (Zurich, Switzerland) which can conduct kinetic measurements over a time course. Rates of ONP formation are determined by a linear regression analysis of an ONP versus time plot.

mRNA Stability Measurements

As a standard control, mRNA stability is measured using Real Time PCR. The amount modified lacZ mRNA will be monitored across all constructs using identical 5′ and 3′ primers.

Since modifications will be apparent to those of skill in this art, it is intended that this invention be limited only by the scope of the appended claims.

What is claimed is:

1. A method of analyzing translational kinetics of an mRNA into polypeptide encoded by a heterologous gene in a host organism comprising:
(a) providing translational kinetics values for codon pairs in a host organism;
(b) generating a first graphical display of the translational kinetics values of actual codon pairs of an original polypeptide-encoding nucleotide sequence of a heterologous gene as a function of codon position;
(c) providing a modified nucleotide sequence encoding the same polypeptide as the original nucleotide sequence;
(d) generating a second graphical display of the translational kinetics values of the codon pairs of the modified polypeptide-encoding nucleotide sequence as a function of codon position; and
(e) comparing said first and second graphical displays to predict the translational kinetics of the polypeptide encoded by the modified polypeptide-encoding nucleotide sequence relative to the unmodified polypeptide-encoding nucleotide sequence.

2. The method of claim 1, wherein the translational kinetics values are based, at least in part, on normalized chi squared values of observed codon pair frequency versus expected codon pair frequency in the host organism.

3. The method of claim 1, wherein the translational kinetics values are based, at least in part, on an empirical measurement of the translational kinetics of a codon pair in the host organism.

4. The method of claim 1, wherein the translational kinetics values are based, at least in part, on determination of a translational kinetics value that is conserved across two or more species at a boundary location between autonomous folding units of a protein present in the two or more species, wherein the group of two or more species includes the host organism.

5. The method of claim 1, wherein the translational kinetics values are based, at least in part, on determination of a normalized value of observed codon pair frequency versus expected codon pair frequency conserved across two or more species at a boundary location between autonomous folding units of a protein present in the two or more species, wherein the group of two or more species includes the host organism.

6. The method of claim 1, wherein the translational kinetics values are based, at least in part, on determination of a translational kinetics value that is positionally conserved across two or more species for a protein present in the two or more species, wherein the group of two or more species includes the host organism.

7. The method of claim 1, wherein the translational kinetics values are based, at least in part, on determination of a normalized value of observed codon pair frequency versus expected codon pair frequency that is positionally conserved across two or more species for a protein present in the two or more species, wherein the group of two or more species includes the host organism.

8. The method of claim 1, wherein the translational kinetics values are based, at least in part, on determination of a codon pair conserved across two or more proteins of the host organism at boundary locations between autonomous folding units of the two or more proteins.

9. The method of claim 1, wherein the abscissa delineates nucleotide position of a polypeptide-encoding nucleotide sequence.

10. The method of claim 1, wherein the ordinate contains negative and positive values, where the zero value corresponds to the mean chi-squared value of observed versus expected codon pair frequencies for genes native to the host organism.

11. The method of claim 1, wherein the scale of the ordinate is in units of standard deviations.

12. The method of claim 1, wherein the original polypeptide-encoding nucleotide sequence and the modified polypeptide-encoding nucleotide sequence both encode the same amino acid sequence.

13. The method of claim 1, wherein the original polypeptide-encoding nucleotide sequence and the modified polypeptide-encoding nucleotide sequence encode different amino acid sequences.

14. The method of claim 1, wherein the original polypeptide-encoding nucleotide sequence is modified such that the second graphical display contains an additional translational pause site relative to the first graphical display, where the additional translational pause site is located between two autonomous folding units of a protein.

15. The method of claim 1, wherein the original polypeptide-encoding nucleotide sequence is modified such that the second graphical display contains a removed translational pause site relative to the first graphical display, where the removed translational pause site is located within an autonomous folding unit of a protein.

16. The method of claim 1, wherein the original polypeptide-encoding nucleotide sequence is modified such that the second graphical display more closely resembles the translational kinetics of the mRNA into polypeptide in its native host organism.

17. The method of claim 14, wherein the original polypeptide-encoding nucleotide sequence is modified such that the second graphical display contains an additional translational pause site relative to the first graphical display, where the additional translational pause site is present in a graphical display of wild type gene expression in the native host organism.

18. The method of claim 14, wherein the original polypeptide-encoding nucleotide sequence is modified such that the second graphical display contains a removed translational pause site relative to the first graphical display, where the removed translational pause site is absent in a graphical display of wild type gene expression in the native host organism.

19. The method of claim 1, wherein the original polypeptide-encoding nucleotide sequence is modified such that the second graphical display contains substantially no codon pairs that are over-represented by more than 5 standard deviations.

20. The method of claim 1, wherein the original polypeptide-encoding nucleotide sequence is modified such that the second graphical display contains substantially no codon pairs that are over-represented by more than 4 standard deviations.

21. The method of claim 1, wherein the original polypeptide-encoding nucleotide sequence is modified such that the second graphical display contains substantially no codon pairs that are over-represented by more than 3 standard deviations.

22. The method of claim 1, wherein the original polypeptide-encoding nucleotide sequence is modified such that the second graphical display contains substantially no codon pairs that are over-represented by more than 2 standard deviations.

23. The method of claim 1, wherein the translational kinetics values are chi-squared 2 values.

24. The method of claim 1, wherein the translational kinetics values are chi-squared 3 values.

25. The method of claim 1, wherein the translational kinetics values are normalized chi-squared values.

26. The method of claim 1, wherein the original polypeptide-encoding nucleotide sequence is a synthetic gene designed to be formed from a plurality of partially overlapping segments that hybridize under conditions that disfavor hybridization of non-adjacent segments.

27. The method of claim 1, wherein the modified polypeptide-encoding nucleotide sequence is a synthetic gene designed to be formed from a plurality of partially overlapping segments that hybridize under conditions that disfavor hybridization of non-adjacent segments.

28. The method of claim 1, wherein the original polypeptide-encoding nucleotide sequence is modified with reference to the effect of the modification on one or more characteristics selected from the group consisting of melting temperature gap between oligonucleotides of synthetic gene, average codon usage, average codon pair chi-squared frequency, absolute codon usage, absolute codon pair frequency, maximum usage in adjacent codons, occurrence of a Shine-Delgarno sequence, occurrence of 5 consecutive G's or 5 consecutive C's, occurrence of a long exactly repeated subsequence, occurrence of a cloning restriction site, occurrence of a user-prohibited sequence, codon usage of a specific codon above user-specified limit, and occurrence of an out of frame stop codon.

29. A method of analyzing translational kinetics of an mRNA into polypeptide encoded by a gene in a non-native host organism comprising:
(a) providing translational kinetics values for codon pairs in a first host organism;
(b) generating a first graphical display of the translational kinetics values of actual codon pairs provided in (a) for a polypeptide-encoding nucleotide sequence of a gene as a function of codon position, wherein the gene is native to the first host organism;
(c) providing translational kinetics values for codon pairs in a second host organism, wherein the polypeptide-encoding nucleotide sequence of the gene is not native to the second organism;
(d) generating a second graphical display of the translational kinetics values of the codon pairs provided in (c) for the polypeptide-encoding nucleotide sequence of the gene as a function of codon position; and
(e) comparing said first and second graphical displays to predict the translational kinetics in the first host organism relative to the translational kinetics in the second host organism.

30. The method of claim 29, further comprising
(f) modifying the polypeptide-encoding nucleotide sequence of the gene;
(g) generating a third graphical display of the translational kinetics values for the codon pairs of the modified polypeptide-encoding nucleotide sequence of the gene as a function of codon position; and
(h) comparing said first and/or second graphical displays to the third graphical display to predict translational kinetics of the mRNA into polypeptide encoded by the modified polypeptide-encoding nucleotide sequence relative to the unmodified polypeptide-encoding nucleotide sequence.

31. The method of claim 30, wherein the polypeptide-encoding nucleotide sequence is modified such that the second graphical display more closely resembles translational kinetics of the mRNA into polypeptide in its native host organism.

32. The method of claim 31, wherein the original polypeptide-encoding nucleotide sequence is modified such that the second graphical display contains an additional translational pause site relative to the first graphical display, where the additional translational pause site is present in a graphical display of wild type gene expression in the native host organism.

33. The method of claim 31, wherein the original polypeptide-encoding nucleotide sequence is modified such that the second graphical display contains a removed translational pause site relative to the first graphical display, where the removed translational pause site is absent in a graphical display of wild type gene expression in the native host organism.

34. A set of graphical displays of translational kinetics chi-squared values of observed versus expected codon pair frequencies in a host organism plotted as a function of polypeptide-encoding nucleotide sequence, comprising:
(a) a first graphical display of translational kinetics values in a host organism of actual codon pairs of an original polypeptide-encoding nucleotide sequence of a heterologous gene as a function of codon position; and
(b) a second graphical display of the translational kinetics values in the host organism of codon pairs of a modified polypeptide-encoding nucleotide sequence of the heterologous gene as a function of codon position.

35. A method of refining the predictive capability of a translational kinetics value of a codon pair in a host organism, comprising:
(a) providing an initial translational kinetics value based on the value of observed codon pair frequency versus expected codon pair frequency for a codon pair in a host organism;
(b) providing additional translational kinetics data for the codon pair in the host organism; and
(c) modifying the initial translational kinetics value according to the additional codon pair translational kinetics data to generate a refined translational kinetics value for the codon pair in the host organism.

36. The method of claim 35, wherein the additional translational kinetics data are selected from the group consisting of:
(a) normalized chi squared values of observed codon pair frequency versus expected codon pair frequency in the host organism;
(b) an empirical measurement of the translational kinetics of the codon pair in the host organism;
(c) degree of conservation of translational kinetics value across two or more species at a boundary location between autonomous folding units of a protein present in the two or more species, wherein the group of two or more species includes the host organism;
(d) degree of positional conservation of translational kinetics value across two or more species for a protein present in the two or more species, wherein the group of two or more species includes the host organism;
(e) degree of conservation of translational kinetics value across two or more proteins of the host organism at a boundary location between autonomous folding units of the two or more proteins; and
(f) combinations of two or more of (a)-(e).

37. The method of claim 36, wherein the modifying step further comprises modifying the translational kinetics value of a selected codon pair according to two or more types of translational kinetics data.

38. A method of improving the predictive capability of a translational kinetics value of a codon pair in a host organism, comprising:
(a) providing translational kinetics data for the codon pair in the host organism; and
(b) generating a translational kinetics value based, at least in part, on the translational kinetics data provided in (a),
wherein the codon pair translational kinetics data are selected from the group consisting of:
(i) an empirical measurement of the translational kinetics of the codon pair in the host organism;
(ii) degree of conservation of translational kinetics value across two or more species at a boundary location between autonomous folding units of a protein present in the two or more species, wherein the group of two or more species includes the host organism;
(iii) degree of positional conservation of translational kinetics value across two or more species for a protein present in the two or more species, wherein the group of two or more species includes the host organism;
(iv) degree of conservation of translational kinetics value across two or more proteins of the host organism at a boundary location between autonomous folding units of the two or more proteins; and
(v) a combination of two or more of (i)-(iv).

39. The method of claim 38, wherein the translational kinetics value of (ii), (iii) or (iv) is the observed codon pair frequency versus expected codon pair frequency.

40. The method of claim 39, wherein the observed codon pair frequency versus expected codon pair frequency is normalized.

41. A method of analyzing translational kinetics of an mRNA into polypeptide encoded by a heterologous gene in a host organism comprising:
(a) providing the amino acid sequence of a heterologous gene;
(b) identifying amino acid sequences related to the amino acid sequence of the heterologous gene;
(c) aligning the related amino acid sequences with each other and with the amino acid sequence of the heterologous gene;
(d) determining the translational kinetics values of the codon pairs of the nucleotide sequence encoding each of the aligned amino acid sequences;
(e) generating a graphical display reflecting the alignment of the amino acid sequences and reflecting the translational kinetics values of the codon pairs of the nucleotide sequence encoding each of the aligned amino acid sequences; and
(f) identifying one or more locations in the aligned amino acid sequences in which translational kinetics values are conserved over most or all aligned amino acid sequences.

42. The method of claim 41, wherein the identifying step comprises identifying a predicted pause that is conserved over most or all aligned amino acid sequences.

43. A method of generating a graphical display of conserved translational kinetics of related genes comprising:
(a) providing the amino acid sequence of a selected gene;
(b) identifying amino acid sequences related to the amino acid sequence of the heterologous gene;
(c) aligning the related amino acid sequences with each other and with the amino acid sequence of the heterologous gene;
(d) determining the translational kinetics values of the codon pairs of the nucleotide sequence encoding each of the aligned amino acid sequences; and
(e) generating a graphical display reflecting the alignment of the amino acid sequences and reflecting the translational kinetics values of the codon pairs of the nucleotide sequence encoding each of the aligned amino acid sequences.

44. A graphical display generated by the method of claim 43.

45. A graphical display comprising a plurality of related amino acid sequences aligned with each other, wherein the depiction of the amino acid sequences also reflects the translational kinetics values of the codon pairs of the nucleotide sequence encoding the aligned amino acid sequences.
